(19) United States
(12) Patent Application Publication
(10) Pub. No.: US 2023/0218501 A1
(43) Pub. Date: Jul. 13, 2023

(54) COMPOSITIONS AND METHODS FOR APPLICATION OVER SKIN

(71) Applicant: SHISEIDO COMPANY, LTD., Tokyo (JP)

(72) Inventor: Ariya AKTHAKUL, East Windsor, NJ (US)

(21) Appl. No.: 18/000,451

(22) PCT Filed: Jun. 18, 2021
(86) PCT No.: PCT/JP2021/023176

§ 371 Date: Dec. 1, 2022

Related U.S. Application Data
(60) Provisional application No. 63/047,648, filed on Jul. 2, 2020.

(51) Int. Cl. A61K 8/895; A61Q 19/00; A61K 8/891

(52) U.S. Cl. CPC A61K 8/895; A61K 8/891; A61Q 19/00; A61K 2800/81

(57) ABSTRACT

Compositions for application to skin of a subject comprising (a) an unsaturated organopolymer, (b) a hydride functionalized polysiloxane and (c) an environment-responsive agent that is capable of transporting out of the composition or facilitates out-transport of one or more beneficial agents from the composition after the composition is applied to a subject. Alternatively, the compositions comprise (a) a bifunctional organopolysiloxane polymer having one unsaturated group and one hydride and (b) the environment-responsive agent. The environment-responsive agent may be a volatile agent and the compositions can be used to create a thin film on the skin of a subject wherein the volatile agent is capable of transporting out of the film. In one embodiment, the volatile agent is transported out of the composition by convection. In one embodiment, the volatile agent is transported out of the composition by diffusion.

### TECHNICAL FIELD

The present invention relates to compositions, devices and methods for modifying skin function and appearance and protecting skin by the formation of a layer over the skin of a subject that forms quickly and that is thin, durable, non-invasive, easy to use, and with skin-like properties.

### BACKGROUND ART

Compositions and polymer materials suitable for skincare products for cosmetic and therapeutic applications are disclosed in PTL 1 to PTL 7. The synthesis and application of an elastic, wearable crosslinked polymer layer (XPL) that mimics the properties of normal, youthful skin have been described in NPL 1. Provided herein are uses and compositions of thin film for the delivery from the skin into the environment of volatile agents.

### CITATION LIST

### Patent Literature

PTL 1: International Application Publication No. WO2012/030984
PTL 2: International Application Publication No. WO2012/030993
PTL 3: International Application Publication No. WO2013/044098
PTL 4: International Application Publication No. WO2017/083398
PTL 5: International Application No. PCT/JP2019/039031
PTL 6: U.S. Provisional Pat. Application Serial No. 62/833,965
PTL 7: U.S. Provisional Pat. Application Serial No. 62/912,219

### Non-Patent Literature

NPL 1: Yu, Betty, et al. “An elastic second skin,” Nature materials 15.8 (2016): 911

### SUMMARY OF INVENTION

Provided herein is a composition including an environment-responsive agent, wherein the environment-responsive agent is capable of transporting out or facilitates out-transport of one or more beneficial agents. Provided herein is a composition including an environment-responsive agent, wherein the composition can be used to create a thin film on the skin of a subject, and the environment-responsive agent is capable of transporting out of the film or facilitate out-transport of one or more beneficial agents. In one embodiment, the environment-responsive agent is transported out in a controlled manner. In one embodiment, the environment-responsive agent is transported out of the composition by convection. In one embodiment, the environment-responsive agent is transported out of the composition by diffusion. In one embodiment, the environment-responsive agent is a volatile agent. In one embodiment, the environment-responsive agent is fragrance, a volatile active, a drug, a beneficial agent, an active that attracts animals, an active that repels animals, a pheromone, an insect repellent, a mosquito repellent, a cooling agent, a heating agent, an antihistamine, an odor masking active, a humidity control agent, an inhalant, an antidepressant, or nicotine. In one embodiment, the environment-responsive agent is an ambient responsive agent, photo-responsive agent, sound-responsive agent, pressure-responsive agent, heat-responsive agent, water-responsive agent, bodily fluid-responsive agent, saliva-responsive agent, chemical-responsive agent, or electromagnetic wave-responsive agent. In one embodiment, the ambient responsive agent is a volatile agent.

Provided herein is a composition for application to skin of a subject, wherein the composition includes (a) an unsaturated organopolymer; (b) a hydride functionalized polysiloxane; and (c) an environment-responsive agent that is capable of transporting out of the composition or facilitate out-transport of one or more beneficial agents after the composition is applied to a subject. Provided herein is a composition for application to skin of a subject, wherein the composition includes (a) a vinyl functionalized organopolysiloxane; (b) a hydride functionalized polysiloxane; and (c) an environment-responsive agent that is capable of transporting out of the composition or facilitate out-transport of one or more beneficial agents after the composition is applied to a subject. In one embodiment, the composition further includes a catalyst, wherein the catalyst is capable of cross-linking the unsaturated organopolymer or vinyl functionalized organopolysiloxane and the hydride functionalized polysiloxane thereby forming a film over the skin of a subject. Provided herein are compositions for the formation of a film over the skin of a subject, including: a) a bifunctional organopolysiloxane polymer having one unsaturated group and one hydride group; and b) an environment-responsive agent that is capable of transporting out of the composition or facilitate out-transport of one or more beneficial agents from the composition after the composition is applied to a subject. In one embodiment, the composition further includes a catalyst, wherein the catalyst is capable of catalyzing hydrosilylation step-growth polymerization reaction of the bifunctional organopolysiloxane polymer thereby forming a film over the skin of a subject. In one embodiment, the catalyst is a transition metal. In one embodiment, the transition metal is platinum. In one embodiment, the unsaturated organopolymer is vinyl-functionalized organopolymer. In one embodiment, the unsaturated organopolymer is vinyl-functionalized organopolysiloxane.

In one embodiment, provided herein is a kit for the formation of a film over the skin of a subject, wherein the kit includes a) a first container including the composition provided herein; and b) a second container including the catalyst.

In one embodiment, the composition is configured such that the transition metal is prevented from catalyzing the cross-linking reaction or hydrosilylation step-growth polymerization reaction before film-formation is desired (e.g., before application to the skin of a subject) thereby allowing formulation of the catalyst and the functional components in a single composition. In one embodiment, the composition further includes at least one ligand at a concentration sufficient to slow down cross-linking reaction between the unsaturated organopolymer or vinyl functionalized organopolysiloxane and the hydride functionalized polysiloxane, such that these components can be formulated and stored together as a mixture without significant cross-linking. In one embodiment, the composition further includes at least one ligand at a concentration sufficient to slow down the hydrosilylation step-growth polymerization reaction of the bifunctional organopolysiloxane polymer, such that these components can be formulated and stored together as a mixture without significant polymerization.

In one embodiment, the composition further includes at least one encapsulating agent, wherein the encapsulating agent slows down or prohibits cross-linking reaction between the unsaturated organopolymer or vinyl functionalized organopolysiloxane and the hydride functionalized polysiloxane by forming physical or chemical barriers such as microcapsules between the transition metal and hydride functionalized polysiloxane, such that these components can be formulated and stored together as a mixture without significant cross-linking. In one embodiment, the composition further includes at least one encapsulating agent, wherein the encapsulating agent slows down or prohibits the hydrosilylation step-growth polymerization reaction of the bifunctional organopolysiloxane polymer by forming physical or chemical barriers such as microcapsules between the catalyst and bifunctional organopolysiloxane polymer, such that these components can be formulated and stored together as a mixture without significant polymerization.

In one embodiment, the ligand slows down the cross-linking reaction. In one embodiment, the ligand slows down the cross-linking reaction via complexation, or coordination. In one embodiment, the ligand slows down the hydrosilylation step-growth polymerization reaction. In one embodiment, the ligand slows down the hydrosilylation step-growth polymerization reaction via complexation, or coordination. In one embodiment, the ligand is divinyltetramethyldisilane, linear vinyl siloxane, cyclic vinyl siloxane, tris (vinylsiloxy) siloxane, tetrakis (vinylsiloxy) silane, vinyl ketone, vinyl ester, acetylenic alcohol, sulfide, mercaptan, divinyl disiloxane, divinyl trisiloxane, divinyl tetrasiloxane, divinyl dimethicone, 1,5-divinyl-3-phenylpentamethyltrisilxioane, 1,1,5,5-tetramethyl-3,3-diphenyl-1,5-divinyltrisiloxane, trivinyl trimethylcyclotrisiloxane, tetravinyl tetramethylcyclotetrasiloxane, pentavinyl pentamethylcyclopentasiloxane, hexavinyl hexamethylcyclohexasiloxane, tris (vinyldimethylsiloxy) silane, tetrakis (vinyldimethylsiloxy) silane, methacryloxypropyl tris(vinyldimethylsiloxy) silane, dimethyl fumarate, dimethyl maleate, methyl vinyl ketone, methoxy butanone, methyl isobutynol, ethyl mercaptan, diethyl sulfide, hydrogen sulfide, or dimethyl disulfide. In one embodiment, the ligand is divinyltetramethyldisilane, linear vinyl siloxane, cyclic vinyl siloxane, tris (vinylsiloxy) siloxane, or tetrakis (vinylsiloxy) silane. In one embodiment, the ligand is vinyl ketone, vinyl ester, acetylenic alcohol, sulfide, or mercaptan. In one embodiment, the ligand is divinyl disiloxane, divinyl trisiloxane, divinyl tetrasiloxane, or divinyl dimethicone. In one embodiment, the ligand is 1,5-divinyl-3-phenylpentamethyltrisilxoane or 1,1,5,5-tetramethyl-3,3-diphenyl-1,5-divinyltrisiloxane. In one embodiment, the ligand is trivinyl trimethylcyclotrisiloxane, tetravinyl tetramethylcyclotetrasiloxane, pentavinyl pentamethylcyclopentasiloxane, or hexavinyl hexamethylcyclohexasiloxane. In one embodiment, the ligand is tris(vinyldimethylsiloxy) silane, tetrakis (vinyldimethylsiloxy) silane, or methacryloxypropyl tris(vinyldimethylsiloxy) silane. In one embodiment, the ligand is dimethyl fumarate, dimethyl maleate, methyl vinyl ketone or methoxy butanone. In one embodiment, the ligand is methyl isobutynol. In one embodiment, the ligand is ethyl mercaptan, diethyl sulfide, hydrogen sulfide or dimethyl disulfide. In one embodiment, the ligand is alkyl diene, alkyl diyne, alkyl monoyne. In one embodiment, the ligand is butadiene, pentadiene, hexadiene, heptadiene, octadiene. In one embodiment, the ligand is methylbutadiene, methylpentadiene, methylhexadiene, methylheptadience, methyloctadiene. In one embodiment, the ligand is ethylbutadiene, ethylpentadiene, ethylhexadiene, ethylheptadience, ethyloctadiene. In one embodiment, the ligand is dimethylbutadiene, dimethylpentadiene, dimethylhexadiene, dimethylheptadience, dimethyloctadiene, or xylene.

In one embodiment, the encapsulating agent slows down or prohibits the cross-linking reaction. In one embodiment, the encapsulating agent slows down or prohibits the cross-linking reaction by forming physical or chemical barriers between the transition metal and the hydride functionalized polysiloxane. In one embodiment, the encapsulating agent slows down or prohibit the cross-linking reaction by physical or chemical barriers such as microcapsules between the transition metal and the hydride functionalized polysiloxane, wherein the microcapsules have shells formed by the encapsulating agent and cores formed by the transition metal or by the hydride functionalized polysiloxane. In one embodiment, the encapsulating agent slows down or prohibits the hydrosilylation step-growth polymerization reaction. In one embodiment, the encapsulating agent slows down or prohibits the hydrosilylation step-growth polymerization reaction by forming physical or chemical barriers between the transition metal and the bifunctional organopolysiloxane polymer. In one embodiment, the encapsulating agent slows down or prohibit the cross-linking reaction by physical or chemical barriers such as microcapsules between the transition metal and the bifunctional organopolysiloxane polymer, wherein the microcapsules have shells formed by the encapsulating agent and cores formed by the transition metal or by the bifunctional organopolysiloxane polymer. In one embodiment, the encapsulating agent is a polysaccharide, protein, lipid or synthetic polymer. In one embodiment, the encapsulating agent is a polysaccharide, wherein the polysaccharide is gum, starch, cellulose, cyclodextrine or chitosan. In one embodiment, the encapsulating agent is a protein, wherein the protein is gelatin, casein or soy protein. In one embodiment, the encapsulating agent is a lipid, wherein the lipid is wax, paraffin or oil. In one embodiment, the encapsulating agent is a synthetic polymer, wherein the synthetic polymer is an acrylic polymer, polyvinyl alcohol or poly(vinylpyrrolidone), polyester, polyether, polyurethane, polyurea, polyimide, polyamide, polysulfone, polycarbonate, polyphosphate, or their copolymers. In one embodiment, the encapsulating agent is an inorganic material. In one embodiment, the encapsulating agent is an inorganic material, wherein the inorganic material is a silicate, clay or polyphosphate. In one embodiment, the encapsulating agent is a biopolymer or biodegradable polymer. In one embodiment, the encapsulating agent is a biopolymer, wherein the biopolymer is starch. In one embodiment, the encapsulating agent is a biodegradable polymer, wherein the biodegradable polymer is chitosan, hyaluronic acid, cyclodextrin, alginate, an aliphatic polyester or a copolymer of lactic and glycolic acids. In one embodiment, the encapsulating agent is a polyester or polyether or their copolymers. In one embodiment, the encapsulating agent is an aliphatic polyester, wherein the aliphatic polyester is poly(lactic acid). In one embodiment, the encapsulating agent is a copolymer of lactic and glycolic acids, wherein the copolymer of lactic and glycolic acids is poly(lactic co-glycolic acid). In one embodiment, the encapsulating agent is a polyurethane or a polyurea or their copolymers. In one embodiment, the encapsulating agent is polyurethane-1, polyurethane-11, polyurethane-14, polyurethane-6, polyurethane-2, polyurethane-18 or their mixtures thereof. In one embodiment, the encapsulating agent is polyurethane-1. In one embodiment, the encapsulating agent is a self-assembled polymer. In one embodiment, the encapsulating agent is a network-forming inorganic dispersion system. In one embodiment, the encapsulating agent is a network-forming inorganic-organic hybrid system.

In one embodiment, the activity of the ligand to slow down the cross-linking reaction or hydrosilylation step-growth polymerization reaction can be reduced or eliminated by evaporation of the ligand, degradation of the ligand, phase transformation of the ligand, chemical degradation of ligand, deactivation of ligand, use of vibrational energy, or use of electromagnetic waves. In one embodiment, the deactivation of the ligand can be triggered by exposure to a chemical, heat or light. In one embodiment, the chemical is an oxidative agent. In one embodiment, the chemical is a reducing agent. In one embodiment, the oxidative agent is oxygen.

In one embodiment, the activity of the encapsulating agent to slow down or prohibit the cross-linking reaction or hydrosilylation step-growth polymerization reaction can be reduced or eliminated by disassembly of the physical or chemical barriers such as microcapsules. In one embodiment, the activity of the encapsulating agent to slow down or prohibit the cross-linking reaction or hydrosilylation step-growth polymerization reaction can be reduced or eliminated by mechanical action, acoustic, heat, light, dissolution, diffusion, degradation, use of solvents, pH changes, temperature changes, pressure or a combination thereof. In one embodiment, the mechanical action is rubbing. In one embodiment, the heat causes the evaporation of the encapsulating agent. In one embodiment, the activity of the encapsulating agent to slow down or prohibit the cross-linking reaction or hydrosilylation step-growth polymerization reaction can be reduced or eliminated by phase transformation of the encapsulating agent, chemical degradation of the encapsulating agent, deactivation of the encapsulating agent, use of vibrational energy, or use of electromagnetic waves. In one embodiment, the deactivation of the encapsulating agent can be triggered by exposure to a sound, chemical, heat or light. In one embodiment, the chemical is an oxidative agent. In one embodiment, the chemical is a reducing agent. In one embodiment, the oxidative agent is oxygen.

In one embodiment, the unsaturated organopolymer is a vinyl functionalized organopolymer. In one embodiment, the unsaturated organopolymer is an alkene functionalized organopolymer. In one embodiment, the unsaturated organopolymer is an alkyne functionalized organopolymer. In one embodiment, the vinyl functionalized organopolymer is an acrylate organopolymer. In one embodiment, the vinyl functionalized organopolymer is a methacrylate organopolymer. In one embodiment, the vinyl functionalized organopolymer is an acrylic organopolymer. In one embodiment, the vinyl functionalized organopolymer is a methacrylic organopolymer. In one embodiment, the alkene functionalized organopolymer is an organopolymer with diene. In one embodiment, the alkene functionalized organopolymer is an organopolymer with polyene. In one embodiment, the alkyne functionalized organopolymer is an organopolymer with polyyne. In one embodiment, the unsaturated organopolymer is a vinyl functionalized organopolysiloxane.

In one embodiment, the vinyl functionalized organopolysiloxane is a polymer of formula IIa and the hydride functionalized polysiloxane is a polymer of formula III:

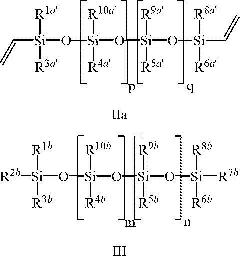

wherein:
R^{1a′}, R^{3a′}, R^{4a′}, R^{5a′}, R^{6a′}, R^{8a′}, R^{9a′} and R^{10a′} are each independently C₁₋₂₀ alkyl, C₂₋₂₀ alkenyl, C₅₋₁₀ aryl, hydroxyl or C₁₋₂₀ alkoxyl;
p and q are each independently an integer from between 10 and 6000;
R^{1b}, R^{2b}, R^{3b}, R^{6b}, R^{7b} and R^{8b} are C₁₋₂₀ alkyl;
R^{4b}, R^{5b}, R^{9b}, R^{10b}, R^{7b} are each independently selected from the group consisting of hydrogen, C₁₋₂₀ alkyl, C₂₋₂₀ alkenyl, C₅₋₁₀ aryl, hydroxyl and C₁₋₂₀ alkoxyl, wherein at least two of R^{4b}, R^{5b}, R^{9b}, R^{10b} are hydrogen; and
m and n are each independently an integer from between 10 and 6000.

In one embodiment, the vinyl functionalized organopolysiloxane is vinyl terminated. In one embodiment, the vinyl functionalized organopolysiloxane is selected from the group consisting of vinyl terminated polydimethylsiloxane; vinyl terminated diphenylsiloxane-dimethylsiloxane copolymers; vinyl terminated polyphenylmethylsiloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethylsiloxane-dimethylsiloxane copolymer; vinylmethylsiloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl gums; vinylmethylsiloxane homopolymers; vinyl T-structure polymers; vinyl Q-structure polymers; monovinyl terminated polydimethylsiloxanes; vinylmethylsiloxane terpolymers; vinylmethoxysilane homopolymers and combinations thereof. In one embodiment, the hydride functionalized polysiloxane is alkyl terminated.

In one embodiment, the hydride functionalized polysiloxane is selected from the group consisting of hydride terminated polydimethylsiloxane; polyphenyl-(dimethylhydrosiloxy)siloxane, hydride terminated; methylhydrosiloxane-phenylmethylsiloxane copolymer, hydride terminated; methylhydrosiloxane-dimethylsiloxane copolymers, trimethylsiloxy terminated; polymethylhydrosiloxanes, trimethylsiloxy terminated; polyethylhydrosiloxane, triethylsiloxane, methylhydrosiloxanephenyloctylmethylsiloxane copolymer; methylhydrosiloxane-phenyloctylmethylsiloxane terpolymer and combinations thereof. In one embodiment, the hydride functionalized polysiloxane includes trimethylsiloxy terminated methylhydrosiloxane-dimethylsiloxane copolymers. In one embodiment, the hydride functionalized polysiloxane has a percent SiH content of between about 3 and about 100%; or a SiH content of between about 0.5 and about 10 mmol/g; or a combination of both. In one embodiment, the hydride functionalized polysiloxane has a viscosity of about 1 to about 11,000 cSt or cP at about 25° C. In one embodiment, the hydride functionalized polysiloxane has at least 2 Si-H units on average.

In one embodiment, the bifunctional organopolysiloxane polymer is a linear siloxane polymer. In one embodiment, the bifunctional organopolysiloxane polymer is a branched siloxane polymer. In one embodiment, the unsaturated group or the hydride group are terminal groups. In one embodiment, the linear siloxane polymer has a degree of polymerization of at least 20 and a dispersity index less than about 1.2, and wherein a ratio of unsaturated terminal groups to hydride terminal groups is substantially 1:1. In one embodiment, the degree of polymerization is about 20 to about 200. In one embodiment, the unsaturated terminal group is selected from the group consisting of vinyl, styryl, allyl, methallyl, hexenyl, octenyl and alkynyl. In one embodiment, the bifunctional organopolysiloxane polymer has a siloxane backbone selected from the group consisting of diphenylsiloxane, phenylmethylsiloxane, trifluoropropylmethylsiloxane, dimethylsilylethylsiloxane, and alkylmethylsiloxane. In one embodiment, the siloxane backbone is dimethylsiloxane and the unsaturated group is vinyl. In one embodiment, the film has no apparent crosslinking. In one embodiment, the linear siloxane polymer is a monovinyl-monohydride terminated polysiloxane. In one embodiment, the film is formed via hydrosilylation step-growth polymerization of the linear siloxane polymer. In one embodiment, the linear siloxane polymer is capable of being reacted with a metal catalyst to form the film over the subject’s skin. In one embodiment, the linear siloxane polymer is a monovinyl-monohydride terminated polydimethylsiloxane.

In one embodiment, provided herein is a composition for application to skin of a subject, wherein the composition includes (a) an unsaturated organopolymer; (b) a hydride functionalized polysiloxane; and (c) an environment-responsive agent that is capable of transporting out of the composition or facilitate out-transport of one or more beneficial agents from the composition after the composition is applied to a subject. In one embodiment, provided herein is a composition for application to skin of a subject, wherein the composition includes (a) a vinyl functionalized organopolysiloxane; (b) a hydride functionalized polysiloxane; and (c) a volatile agent that is capable of transporting out of the composition after the composition is applied to a subject. In one embodiment, provided herein is a composition for application to skin of a subject, wherein the composition includes: a) a bifunctional organopolysiloxane polymer having one unsaturated group and one hydride group; and b) an environment-responsive agent that is capable of transporting out of the composition or facilitate out-transport of one or more beneficial agents from the composition after the composition is applied to a subject. In one embodiment, provided herein is a composition for application to skin of a subject, wherein the composition includes: a) a bifunctional organopolysiloxane polymer having one unsaturated group and one hydride group; and b) a volatile agent that is capable of transporting out of the composition after the composition is applied to a subject. In one embodiment, the environment-responsive agent is an ambient responsive agent, photo-responsive agent, sound-responsive agent, pressure-responsive agent, heat-responsive agent, or electromagnetic wave-responsive agent. In one embodiment, the environment-responsive responsive agent is a volatile agent. In one embodiment, the ambient responsive agent is a volatile agent.

### BRIEF DESCRIPTION OF DRAWINGS

FIG. 1 depicts a scheme of a microcapsule.

FIG. 2 depicts the morphology of microcapsules.

FIG. 3 depicts a schematic overview over the four principal process steps in microsphere preparation by solvent extraction/evaporation.

FIG. 4 depicts a schematic illustration of the process of micro-encapsulation by spray-drying.

### DESCRIPTION OF EMBODIMENTS

As used herein, the term “skin” includes body surfaces where normal skin is intact, compromised, or partially or completely lost or removed. Skin further includes skin imperfections that are commonly considered to be part of “skin.” Examples of skin imperfections include wrinkles, blemishes, freckles, acne, moles, warts, lesions, scars, tattoos, bruises, skin disfigurements, birth marks, sun damage, age damage, spots (e.g., aging spots), uneven skin tone, sagging skin, cellulite, stretch marks, loss of skin elasticity, skin roughness, enlarged pores, hyperpigmentation, telangiectasia, redness, shine, port wine stain (or nevus flammeus, e.g., nevus flammeus nuchae or midline nevus flammeus), and melasma. Skin further includes skin area over which any cosmetic, personal care, medical, paint, or any other foreign material, or a combination thereof, is applied.

As used herein, the term “layer” includes a covering, film, sheet, barrier, coating, membrane, device or prosthetic skin formed on, sprayed on, or spread over a surface. A layer may be, but is not necessarily, continuous. A layer may, but does not necessarily, have substantially even and/or uniform thickness.

As used herein, the terms “compromised skin barrier function,” “compromised skin barrier,” or “compromised skin condition” include conditions such as dermatological disorders, skin conditions, and wounds.

As used herein, the term “dermatological disorders” include disorders that cause at least one symptom on the skin of a subject that may require medical treatment. Dermatological disorders may be caused by, among other things, autoimmune disorders and/ or environmental factors, such as allergens or chemicals. Examples of symptoms of dermatological disorders include, but are not limited to, itchy skin, dry skin, crusting, blistering, or cracking skin, dermatitis, skin edema, or skin lesion formation. Dermatological disorders include, but are not limited to, eczema, psoriasis, ichthyosis, rosacea, chronic dry skin, cutaneous lupus, lichen simplex chronicus, xeroderma, acne, disease-driven secondary dermatological disorder, and ulcer.

As used herein, ichthyosis includes, e.g., ichthyosis vulgaris, acquired ichthyosis, X-linked ichthyosis, congenital ichthyosiform erythroderma, nonbullous (nbCIE), epidermolytic hyperkeratosis (bullous ichthyosis, bCIE), Harlequin type ichthyosis, ichthyosis bullosa of Siemens, ichthyosis hystrix, Curth-Macklin type, Hystrix-like ichthyosis with deafness, Lamellar ichthyosis, type 1, Lamellar ichthyosis, type 2, Lamellar ichthyosis, type 3, Lamellar ichthyosis, type 4, Lamellar ichthyosis, type 5, CHILD Syndrome, Conradi-Hunermann syndrome, ichthyosis follicularis with alopecia and photophobia syndrome, Keratitis-ichthyosis-deafness syndrome, Netherton syndrome, Neutral lipid storage disease with ichthyosis, adult Refsum disease, ichthyosis and male hypogonadism, Sjogren-Larsson syndrome, and photosensitive trichothiodystrophy (IBIDS syndrome). As used herein, rosacea includes, e.g., erythematotelangiectatic rosacea, papulopustular rosacea, phymatous rosacea (e.g., rhinophyma), and granulomatous rosacea.

As used herein, cutaneous lupus includes, e.g., acute cutaneous lupus, subacute cutaneous lupus, chronic cutaneous lupus, chilblain lupus erythematosus, discoid lupus erythematosus, lupus erythematosus-lichen planus overlap syndrome, lupus erythematosus panniculitis, tumid lupus erythematosus and verrucous lupus erythematosus.

As used herein, acne includes, e.g., acne vulgaris, acne aestivalis, acne conglobate, acne cosmetic, acne fulminans, acne keloidalis nuchae, acne mechanica, acne medicamentosa (also known as drug-induced acne, e.g., steroid acne), acne miliaris necrotica, acne necrotica, acne rosacea, and hidradenitis suppurativa.

As used herein, the term “disease-driven secondary dermatological disorder” refers to a dermatological condition that may require treatment and was caused by or is associated with a non-dermatological disorder. A “non-dermatological disorder” includes disorders not primarily associated with the skin but which may result in, be associated with, or have a secondary manifestation of a skin condition, for example, a disorder of the circulatory system or metabolism of the subject. Disease-driven secondary dermatological disorders include, for example, an ulcer caused by diabetes mellitus (e.g., diabetic foot ulcer), a bacterial, viral or fungal infection, cancer, pressure (e.g., a bedsore), blood disorders, conditions affecting the nervous system (e.g., neuropathic ulcers (also known as “mal perforans”)), conditions affecting the nervous system (e.g., arterial insufficiency ulcers (also known as “ischemic ulcers”) or vascular ulcers), and/ or a chronic wound.

As used herein, the term “skin conditions” include, but are not limited to, itchy skin, raw skin, dry skin, flaking or peeling skin, blisters on the skin, redness, swelling or inflammation of the skin, and oozing, scabbing or scaling skin. Skin conditions also include compromised skin barrier conditions caused by laser, light or chemical peel treatment.

As used herein, the term “wounds” include injuries to the skin wherein the skin is torn, cut or punctured. Wounds include open wounds, for example, abrasions, lacerations, incisions, punctures, avulsions, or amputations. Wounds also include burn wounds, a type of injury to skin and/or flesh caused by heat, electricity, wind, chemicals, light, radiation or friction.

As used herein, the terms “treat,” “treating” and “treatment” include both therapeutic and prophylactic / preventative measures. “Treat,” “treating” and “treatment” further include both disorder modifying treatment and symptomatic treatment. Treatment may ameliorate or cause a reduction in the severity and/or duration of at least one symptom of the conditions of compromised skin barrier function. Treatment may also cause a complete recovery from the conditions of compromised skin barrier function.

As used herein, the terms “apply,” “applied” and “application” includes any and all known methods of contacting or administering compositions provided herein to a subject’s skin or body. The application may be by finger, hand, brush, cotton ball, cotton swab, tissue, pad, sponge, roll-on, spatula, dispenser, drops, spray, splash, foam, mousse, serum, spritz, and other appropriate methods.

As used herein, the term “subject” includes subjects in which the compositions disclosed herein would be appropriate for use, particularly animals (e.g., a human). Subjects may further include plants, wherein skin refers to the surface over portions of the plant that may benefit from application of the composition, such as flowers, leaves, fruits, stems, branches, bark, and roots.

As used herein, the term “In vitro” means tested or formed not on, in, or over a subject’s skin or body.

As used herein, the term “routine daily activities” includes instrumental activities of daily living, such as feeding (e.g., eating, drinking, taking medications), continence (e.g., urination and defecation), toileting, dressing, bathing (e.g., shower, bath), grooming, physical ambulation (e.g., walking, using transportation), talking (e.g., using the telephone), preparing food, housekeeping, doing laundry, shopping, and handling finances. Examples of such daily activities are described in Lawton and Brody, Assessment of older people: self-maintaining and instrumental activities of daily living, Gerontologist 1969 Autumn;9(3):179-86 and Katz et al., Studies of Illness in the Aged. The Index of ADL: A Standardized Measure of Biological and Psychosocial Function, JAMA 1963 Sep 21;185:914-9.

As used herein, the term “demanding activities” includes activities that generate elevated level of strain and/or stress on the skin of a subject as compared to the strain or stress generated by routine daily activities. Examples of such demanding activities include exercising, swimming (in sea-water, fresh water or chlorinated water), steam room (heat at high humidity), sauna (heat at low humidity), and other like activities.

Unless otherwise stated, descriptions of any material used as part of any composition disclosed herein are of such material as an ingredient of the composition prior to mixing, combination and/or reaction of such material with other ingredient(s) of the composition.

As used herein, the term “crosslinkable polymer” refers to a polymer that can physically or chemically interact, or both physically and chemically interact, with itself or with other polymers to form a layer on a surface (e.g., skin, leather, glass, plastic, metal) to which it is applied. “Physically interact” refers to the formation of noncovalent interaction (e.g., hydrogen bonds, or electrostatic, polar, ionic, van der Waals, or London forces) between two or more polymer chains. “Chemically interact” refers to the formation of covalent bonds between two or more polymer chains. Covalent bonds may be formed through chemical reactions that occur spontaneously or are initiated by, for example, catalyst, moisture, heat, pressure, change in pH, or radiation. The crosslinkable polymer(s) may be homopolymer or copolymer, for example, random copolymer, alternating copolymer, periodic copolymer, statistical copolymer, block copolymer, graft or grafted copolymer, or a combination thereof. The crosslinkable polymer(s) may be a linear polymer, a branched polymer, a star polymer, a loop polymer, or a combination thereof. In one embodiment, the crosslinkable polymer is polyglycerin. In one embodiment, the crosslinkable polymer is KSG-710. In one embodiment, the crosslinkable polymer is KSG-710 supplied by Shin-Etsu.

In preferred embodiments, the composition comprises one or more organopolymer(s). An “organopolymer” refers to a polymer that includes carbon. In preferred embodiments, the organopolymer is an organopolysiloxane polymer. In preferred embodiments, the organopolysiloxane polymer is a linear siloxane polymer. In preferred embodiments, the organopolysiloxane polymer is a branched siloxane polymer. In preferred embodiments, the organopolymer is a bifunctional organopolysiloxane polymer. In preferred embodiments, the bifunctional organopolysiloxane polymer is a linear siloxane polymer. In preferred embodiments, the bifunctional organopolysiloxane polymer is a branched siloxane polymer.

The term “viscosity” refers to the measure of the resistance of a fluid which is being deformed by either shear stress or tensile stress. The viscosity of the composition affects the thickness, spreadability, and evenness and/or uniformity of the layer formed on a substrate. Viscosity may be reported as either dynamic viscosity (also known as absolute viscosity, typical units Pas, Poise, P, cP) or kinematic viscosity (typical units cm²/s, Stokes, St, cSt), which is the dynamic viscosity divided by density of the fluid measured. Viscosity ranges of the ingredients disclosed herein are commonly provided by the supplier of the ingredients in units of kinematic viscosity (e.g., cSt), as measured using a Rheometer or a Cannon-Fenske Tube Viscometer.

Viscosity of a fluid can be measured in vitro, for example, using a rheometer (e.g., linear shear rheometer or dynamic shear rheometer) or a viscometer (also called viscosimeter, e.g., capillary viscometer or rotational viscometer), at an instrument specific strain. For example, Thomas G. Mezger, The Rheology Handbook: For Users of Rotational and Oscillatory Rheometers (2nd Ed.), Vincentz Network, 2006, and American Society for Testing and Materials (ASTM) standards such as ASTM D3835-08, ASTM D2857-95, ASTM D2196-10, and ASTM D2983-09 provide instructions on how to measure the viscosity of a fluid. Viscosity of a fluid is preferably measured in vitro using the Rheometer Viscosity Measurement Test described herein. Density of the fluid may vary with temperature or pressure. Unless otherwise specified, all properties of compositions, layers and/or devices disclosed herein, including viscosity, are measured at room temperature (about 25° C.) and about 1 atmosphere air pressure.

Anhydrous compositions generally have longer shelf-life than emulsions with similar ingredients, without the need for preservatives against bacteria or mold. “Anhydrous” as used herein refers to containing as an ingredient less than about 10%, less than about 5%, less than about 2%, less than about 1%, or less than about 0.1% water. In some embodiments, the composition is anhydrous. In some embodiments, the composition is an emulsion. In some embodiments, the composition is a dispersion. In some embodiments, the composition is a suspension. In some embodiments, the composition is a paste. In some embodiments, the composition is a semi-solid. In some embodiments, the composition is an ointment. In some embodiments, the composition is a cream. In some embodiments, the composition is a serum. In some embodiments, the composition is a lotion. In some embodiments, the composition is a patch. In certain embodiments, the composition can be spread, sprayed, stenciled stamped, patterned, patched, transferred, layered, covered or spritzed over skin.

The term “glass transition temperature” refers to the temperature at a transition from the solid state to the liquid state occurs. A glass transition temperature may be reported as a temperature (°C, °F or K). Glass transition temperature can be measured in vitro, for example, using thermal analysis instruments such as a Differential Scanning Calorimeter (DSC) or a Thermogravimetric Analysis (TGA).

The term “tack-free time” refers to the time when the layer has solidified sufficiently that it no longer sticks to a finger or a substrate that lightly touches it under normal force less than 0.15 Newtons, incurring stickiness to the film.

The term “adhesive force” refers to the force per unit length required to separate the materials adhered to a standard substrate such as leather or polypropylene or polyurethane. In certain embodiments, the adhesive force of the layer on polypropylene substrate is greater than about 2 N/m.

The terms “tensile strength,” or “ultimate tensile strength,” or “fracture stress,” or “stress at break,” or “maximum tensile stress,” or “ultimate tensile stress,” or “fracture strength,” or “breaking strength” refer to stress at which a specimen fails via fracture. Tensile strength can be measured on a specimen formed from the composition in vitro, for example, using the Cyclic and Extension Pull Test as described herein.

The terms “fracture strain,” or “elongation at break,” or “stretchiness at break,” or “strain at break,” or “maximum elongation,” or “maximum strain,” or “maximum stretchiness” or “extension at break” or “maximum extension” refer to strain at which a specimen fails via fracture. Fracture strain can be measured on a specimen formed from the composition in vitro, for example, using the Cyclic and Extension Pull Test as described herein.

The terms “tensile modulus,” or “Young’s modulus,” or “modulus of elasticity,” or “stiffness,” or “tensile stiffness,” or “elastic modulus” refer to the force per unit area that is needed to stretch and deform a material beyond the initial length. Tensile modulus is an inverse of compliance, relating to flexibility or deformability of a material beyond the initial length. Tensile modulus can be measured on a specimen formed from the composition in vitro, for example, using the Cyclic and Extension Pull Test as described herein. Tensile modulus can also be measured using the ASTM D5083 Tensile Properties of Reinforced Thermosetting Plastics Using Straight-Sided Specimens standard test.

The terms “shear modulus” or “modulus of rigidity” or “shear stiffness” refer to the force per unit area that is needed to shear and deform a material beyond the initial length. Shear modulus is be measured on a specimen formed from the composition in vitro by using the ASTM D7175 Determining the Rheological Properties of Asphalt Binder using a Dynamic Shear Rheometer.

The term “cyclic tensile residual strain” refers to tensile residual strain after cyclic tensile deformation. The term “residual strain” refers to strain that remains in a material after the original cause of stress has been removed. Residual strain may be reported as plastic strain, inelastic strain, non-elastic strain, or viscoelastic strain. The cyclic tensile residual strain can be measured on a specimen formed from the composition in vitro, for example, using the Cyclic and Extension Pull Test as described herein.

The terms “cyclic tensile hysteresis loss energy” or “cyclic hysteresis strain energy” refer to the excess energy being dissipated as heat when the specimen is subjected to cyclic tensile deformation. Cyclic tensile hysteresis loss energy can be measured on a specimen formed from the composition in vitro, for example, using the Cyclic and Extension Pull Test as described herein.

The terms “fracture toughness,” or “toughness,” or “tensile toughness,” or “deformation energy,” or “failure energy,” or “fracture energy” refer to the ability to absorb energy of mechanical deformation per unit volume up to the point of failure. Fracture toughness can be measured on a specimen formed from the composition in vitro, for example, using the Cyclic and Extension Pull Test as described herein.

The term “oxygen transmission rate” or OTR refers to the permeation flux of oxygen through a membrane with certain thickness. Oxygen transmission rate can be measured on a specimen formed from the composition in vitro, for example, using the ASTM F2622 Oxygen Gas Transmission Rate Through Plastic Film and Sheeting Using Various Sensors test.

The term “oxygen permeance” refers to the permeation flux of oxygen through a membrane with certain thickness, per unit oxygen vapor pressure difference between the membrane (typically in cmHg). Oxygen permeance can be measured on a specimen formed from the composition in vitro, for example, using the ASTM F2622 Oxygen Gas Transmission Rate Through Plastic Film and Sheeting Using Various Sensors test.

The terms “oxygen permeability coefficient” or “intrinsic oxygen permeability” refer to a measure of how fast the oxygen can move through a membrane, which involves a successive process of oxygen sorption into a membrane then followed by oxygen diffusion through the membrane. Oxygen permeability coefficient can be measured on a specimen formed from the composition in vitro, for example, using the ASTM F2622 Oxygen Gas Transmission Rate Through Plastic Film and Sheeting Using Various Sensors test.

The term “water vapor transmission rate” or WVTR refers to the permeation flux of water vapor through a membrane with certain thickness. Water vapor transmission rate can be measured on a specimen formed from the composition in vitro, for example, using the ASTM F1249 Water Vapor Transmission Rate Through Plastic Film and Sheeting Using a Modulated Infrared Sensor test.

The term “water vapor permeance” refers to the permeation flux of water vapor through a barrier with certain thickness, per unit water vapor pressure difference between one side and the other side of the barrier (typically in cmHg). Water vapor permeance can be measured on a specimen formed from the composition in vitro, for example, using the ASTM F1249 Water Vapor Transmission Rate Through Plastic Film and Sheeting Using a Modulated Infrared Sensor test.

The terms “water vapor permeability coefficient” or “intrinsic water vapor permeability” refer to a measure of how fast water vapor can move through a barrier, which involves a successive process of water vapor sorption into a barrier, followed by water vapor diffusion through the barrier. Water vapor permeability coefficient can be measured on a specimen formed from the composition in vitro, for example, using the ASTM F1249 Water Vapor Transmission Rate Through Plastic Film and Sheeting Using a Modulated Infrared Sensor test.

The term “transepidermal water loss” refers to the measurement of the quantity of water that passes from inside a body through the epidermal layer to the surrounding atmosphere via diffusion and evaporation processes. Transepidermal water loss is measured by using the Transepidermal Water Loss (TEWL) Measurement Test as described herein. Differences in TEWL measurements caused by age, race, gender, and/or area of the skin of the subject tested are generally less than the standard error in the TEWL measurements.

The term “skin hydration” refers to the measure of water content of the skin, typically through a Corneometer which is based on capacitance measurement of a dielectric medium near skin surface.

The term “retraction time” refers to the time taken for the skin to return to its original state after initial deformation by the Suction Cup device. Skin retraction time can be measured, for example, using a cutometer/suction cup pursuant to the procedure as described in H. Dobrev, “Use of Cutometer to assess epidermal hydration,” Skin Research and Technology 2000, 6(4):239-244.

As used herein, and unless otherwise specified, the term “about,” when used in connection with doses, amounts, or weight percent of ingredients of a composition or a dosage form, means dose, amount, or weight percent that is recognized by those of ordinary skill in the art. Specifically, the term “about” contemplates a dose, amount, or weight percent within 30%, 25%, 20%, 15%, 10%, or 5% of the specified dose, amount, or weight percent is encompassed.

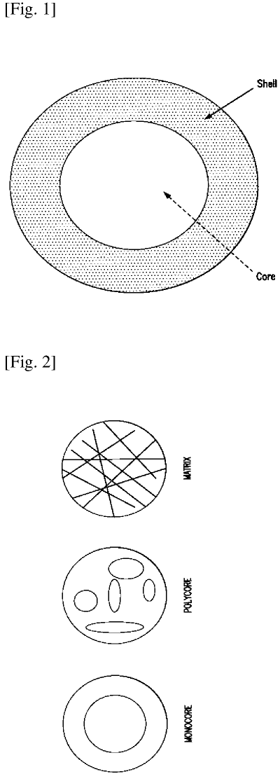

The term “encapsulation” refers to a process of encapsulating a material (core) in a shell of a second material (shell/wall material), permanently or temporarily. In some embodiments, the second material is called “encapsulating agent.” The process results in small capsules as described in FIG. 1, termed microcapsules. Microcapsules may be classified as mononuclear, polynuclear or matrix type as described in FIG. 2. In some embodiments, the microcapsules have diameters between one micron and a few millimeters. In some embodiments, the microcapsules whose diameters are between about 50 nm to about 2 mm. In some embodiments, the microcapsules whose diameters are between about 2 µm to about 2000 µm. In some embodiments, the microcapsules whose diameters are between about 50 nm to about 1000 nm. In some embodiments, the microcapsules whose diameters are between about 100 nm to about 500 nm. In some embodiments, the microcapsules whose diameters are in the nanometer range are referred to as nanocapsules.

The term “environment-responsive agent” refers to an agent which is capable of transporting out or facilitate out-transport of one or more beneficial agents. In one embodiment, an environment-responsive agent is an agent which is capable of transporting out or facilitate out-transport of one or more beneficial agents in response to environmental changes. In one embodiment, an environment-responsive agent is an agent which is capable of transporting out or facilitate out-transport of one or more beneficial agents from a film over the skin of a subject in response to environmental changes. In one embodiment, the environment-responsive agent is an ambient responsive agent, photo-responsive agent, sound-responsive agent, pressure-responsive agent, heat-responsive agent, water-responsive agent, bodily fluid-responsive agent, saliva-responsive agent, chemical-responsive agent, or electromagnetic wave-responsive agent. In one embodiment, the environment-responsive responsive agent is a volatile agent. In one embodiment, the ambient responsive agent is a volatile agent.

The term “volatile agent” refers to chemicals that have a high vapor pressure at ordinary room temperature, which causes large numbers of molecules of the chemical to evaporate or sublimate and enter the surrounding air. A volatile agent may be in a solid, liquid or gas form at 25° C. under 1 standard atmospheric pressure.

The term “hydrosilylation reaction” refers to cross-linking reaction or hydrosilylation step-growth polymerization reaction.

### 1. Detailed Description

Provided herein are methods for delivering “out” from the skin of subject into the environment of specified environment-responsive agents. Examples of such environment-responsive agents are provided in section 1.5. Compositions for such uses are also provided. Specifically, thin films described in International Application No. PCT/JP2019/039031 and U.S. Provisional Pat. Application Serial Nos. 62/833,965 and 62/912,219 can be formulated to futher include environment-responsive agents. Such thin films can be formulated as two-step systems (see section 1.1 below) or single step systems (see section 1.12 below). The environment-responsive agent can be formulated in step 1 or step 2 of the two-step system. The formulation of the first step of the two-step system includes the unsaturated organopolymer or vinyl functionalized organopolysiloxane and the hydride functionalized polysiloxane as described herein or the bifunctional organopolysiloxane polymer as described herein. More details about these compoentns are provided in sections 1.1 to 1.4, etc.

Provided herein is a composition including an environment-responsive agent, wherein the environment-responsive agent is capable of transporting out or facilitates out-transport of one or more beneficial agents. Provided herein is a composition including an environment-responsive agent, wherein the composition can be used to create a thin film on the skin of a subject, and the environment-responsive agent is capable of transporting out or facilitates out-transport of one or more beneficial agents from the film. In one embodiment, the environment-responsive agent is transported out or facilitate out-transport of one or more beneficial agents in a controlled manner.

### 1.1 Thin Films With Environment-Responsive Agents

Provided herein is a composition for application to skin of a subject, wherein the composition includes (a) an unsaturated organopolymer; (b) a hydride functionalized polysiloxane; and (c) an environment-responsive agent that is capable of transporting out of the composition after the composition is applied to a subject. Provided herein is a composition for application to skin of a subject, wherein the composition includes (a) a vinyl functionalized organopolysiloxane; (b) a hydride functionalized polysiloxane; and (c) a volatile agent that is capable of transporting out of the composition after the composition is applied to a subject. In one embodiment, the composition further includes a catalyst, wherein the catalyst is capable of cross-linking the unsaturated organopolymer or vinyl functionalized organopolysiloxane and the hydride functionalized polysiloxane thereby forming a film over the skin of a subject. Provided herein are compositions for the formation of a film over the skin of a subject, including: a) a bifunctional organopolysiloxane polymer having one unsaturated group and one hydride group; and b) an environment-responsive agent that is capable of transporting out or facilitate out-transport of one or more beneficial agents from the composition after the composition is applied to a subject. In one embodiment, the composition further includes a catalyst, wherein the catalyst is capable of catalyzing hydrosilylation step-growth polymerization reaction of the bifunctional organopolysiloxane polymer thereby forming a film over the skin of a subject. In one embodiment, the catalyst is a transition metal. In one embodiment, the transition metal is platinum.

In one embodiment, the components provided herein are mixed and stored together as a homogeneous mixture. In one embodiment, the components provided herein are mixed and stored together as a heterogeneous mixture, e.g., a suspension or an emulsion.

In certain embodiments, the composition further includes at least one ligand at a concentration sufficient to slow down cross-linking reaction between the unsaturated organopolymer or vinyl functionalized organopolysiloxane and the hydride functionalized polysiloxane, such that these components can be formulated and stored together as a mixture without significant cross-linking. In one embodiment, the composition further includes at least one ligand at a concentration sufficient to slow down the hydrosilylation step-growth polymerization reaction of the bifunctional organopolysiloxane polymer, such that these components can be formulated and stored together as a mixture without significant hydrosilylation step-growth polymerization.

In certain embodiments, the composition further includes at least one encapsulating agent at a concentration sufficient to slow down or prohibit cross-linking reaction between the unsaturated organopolymer or vinyl functionalized organopolysiloxane and the hydride functionalized polysiloxane, such that these components can be formulated and stored together as a mixture without significant cross-linking. In one embodiment, the composition further includes at least one encapsulating agent, wherein the encapsulating agent slows down or prohibits the hydrosilylation step-growth polymerization reaction of the bifunctional organopolysiloxane polymer by forming physical or chemical barriers such as microcapsules between the catalyst and bifunctional organopolysiloxane polymer, such that these components can be formulated and stored together as a mixture without significant hydrosilylation step-growth polymerization.

In certain embodiments, the compositions for use with the methods provided herein include a catalyst; at least one environment-responsive agent; at least one ligand; at least one unsaturated organopolymer; and at least one hydride functionalized polysiloxane. In certain embodiments, the compositions for use with the methods provided herein include a catalyst; at least one volatile agent; at least one ligand; at least one vinyl functionalized organopolysiloxane; and at least one hydride functionalized polysiloxane. In certain embodiments, the compositions for use with the methods provided herein include a catalyst; at least one environment-responsive agent; at least one encapsulating agent; at least one unsaturated organopolymer; and at least one hydride functionalized polysiloxane. In certain embodiments, the compositions for use with the methods provided herein include a catalyst; at least one volatile agent; at least one encapsulating agent; at least one vinyl functionalized organopolysiloxane; and at least one hydride functionalized polysiloxane.

In certain embodiments, the compositions for use with the methods provided herein include a catalyst; at least one environment-responsive agent; at least one ligand; and at least one bifunctional organopolysiloxane polymer. In certain embodiments, the compositions for use with the methods provided herein include a catalyst; at least one volatile agent; at least one ligand; and at least one bifunctional organopolysiloxane polymer. In certain embodiments, the compositions for use with the methods provided herein include a catalyst; at least one environment-responsive agent; at least one encapsulating agent; and at least one bifunctional organopolysiloxane polymer. In certain embodiments, the compositions for use with the methods provided herein include a catalyst; at least one volatile agent; at least one encapsulating agent; and at least one bifunctional organopolysiloxane polymer.

In certain embodiments, the composition is a two-part composition, e.g., the first part and the second part. In certain embodiments, the first part and the second part are applied either one at a time or in combination to form the layer. In certain embodiments, the first part includes the unsaturated organopolymer and the hydride functionalized polysiloxane as described herein. In certain embodiments, the first part includes the vinyl functionalized organopolysiloxane and the hydride functionalized polysiloxane as described herein. In certain embodiments, the second part includes the unsaturated organopolymer as described herein. In certain embodiments, the second part includes the vinyl functionalized organopolysiloxane as described herein.

In certain embodiments, the composition is a two-part composition, e.g., the first part and the second part. In certain embodiments, the first part and the second part are applied either one at a time or in combination to form the layer. In certain embodiments, the first part includes the bifunctional organopolysiloxane polymer as described herein.

### 1.2 Unsaturated Organopolymer

In one embodiment, the unsaturated organopolymer is a vinyl functionalized organopolysiloxane. In one embodiment, the vinyl functionalized organopolysiloxanes provided herein is or includes at least one or more compounds of Formula I:

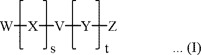

wherein
W is R¹R²R³SiO—, —OR⁴, —NR⁵R⁶, —CR⁷R⁸R⁹ or C₅₋₁₀ aryl;
X is —R¹¹R¹²Si—O—, —OCONR¹³—, —NR¹⁴CONR¹⁵—, —CO—, —NR¹⁶CO—, —SO₂—, —O—, —S— or —NR¹⁷—;
V is absent, C₁₋₂₀ alkyl, C₂₋₂₀ alkenyl, C₅₋₁₀ aryl, —O—, —NR¹⁰— or —S—;
Y is —R¹⁸R¹⁹Si—O—, —OCONR²⁰—, —NR²¹CONR²²—, —CO—, —NR²³CO—, —SO₂—, —O—, —S— or —NR²⁴;
Z is -SiR²⁵R²⁶R²⁷, -OR²⁸, -NR²⁹R³⁰, -CR³¹R³²R³³ or C₅₋₁₀ aryl;
R¹, R², R³, R⁷, R⁸, R⁹ R¹⁰, R¹¹, R¹², R¹⁸ R¹⁹, R²⁵, R²⁶, R²⁷, R³¹, R³² and R³³ are each independently hydrogen, C₁₋₂₀ alkyl, C₂₋₂₀ alkenyl, C₅₋₁₀ aryl, hydroxyl or C₁₋₂₀ alkoxyl;
R⁴, R⁵, R⁶, R¹³, R¹⁴, R¹⁵, R¹⁶, R¹⁷, R²⁰, R²¹, R²², R²³, R²⁴, R²⁸, R²⁹ and R³⁰ are each independently hydrogen, C₁₋₂₀ alkyl, C₂₋₂₀ alkenyl, C₅₋₁₀ aryl; and
s and t are each independently an integer from about 0 to about 6000.

In some embodiments, the composition includes more than one compound of formula I and the compounds of formula once may be the same or different.

X and Y of formula I represent an independent “repeat unit.” The number of X and Y repeat units present in formula I is provided by the value of s and t, respectively. Representative repeat units include:

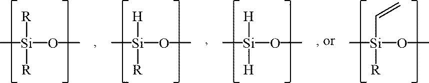

where R is as for defined for R¹, R², R³, etc, above.

It is understood that when more than one X (or Y) repeat unit is present (e.g. s (or t) is more than one), the values for R¹¹, R¹², R¹³, R¹⁴, R¹⁵, R¹⁶, R¹⁷, R¹⁸ R¹⁹, R²⁰, R²¹, R²², R ²³, and R²⁴ are selected independently for each individual repeat unit described by —[X]ₛ — (or —[Y]ₜ—). For example, if the value of the repeat unit X is —R¹¹R¹²Si—O— and the value of s is 3, then —[X]ₛ— is: —[R¹¹R¹²Si—O—R¹¹R¹²Si—O—R¹¹R¹²Si—O]—.

In this example, it is understood that the three R¹¹ groups present in may be the same or different from each other, for example, one R¹¹ may be hydrogen, and the two other R¹¹ groups may be methyl.

W and Z of formula I represent independent terminal caps, one on each end of the polymer. For example, terminal caps include:

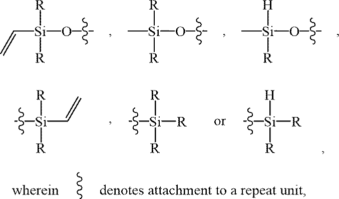

and wherein R is as for defined for R¹, R², R³, etc, above.

In one embodiment,
W is R¹R²R³SiO—, —OR⁴, —NR⁵R⁶, —CR⁷R⁸R⁹ or C₅₋₁₀ aryl;
X is —R¹¹R¹²Si—O—, or —NR¹⁴CONR¹⁵—;
V is absent, C₁₋₂₀ alkyl, C₂₋₂₀ alkenyl, C₅₋₁₀ aryl, —O—, —NR¹⁰— or —S—;
Y is —R¹⁸R¹⁹Si—O—, or —NR²¹CONR²²—;
Z is -SiR²⁵R²⁶R²⁷, —OR²⁸, -NR²⁹R³⁰, -CR³¹R³²R³³ or C₅₋₁₀ aryl;
R¹, R², R³, R⁷, R⁸, R⁹, R¹¹, R¹², R¹⁸ R¹⁹, R²⁵, R²⁶, R²⁷, R³¹, R³² and R³³ are each independently hydrogen, C₁₋₂₀ alkyl, C₂₋₂₀ alkenyl, C₅₋₁₀ aryl, hydroxyl or C₁₋₂₀ alkoxyl;
R⁴, R⁵, R⁶, R¹⁴, R¹⁵, R²¹, R²², R²⁸, R²⁹ and R³⁰ are each independently hydrogen, C₁₋₂₀ alkyl, C₂₋₂₀ alkenyl, C₅₋₁₀ aryl; and
s and t are each independently an integer from about 0 to about 6000, wherein the sum of s and t is not 0.

In one embodiment,
W is R¹R²R³SiO—, —CR⁷R⁸R⁹ or C₅₋₁₀ aryl;
X is —R¹¹R¹²Si—O—, or —NR¹⁴CONR¹⁵—;
V is absent, C₁₋₂₀ alkyl, C₂₋₂₀ alkenyl, or C₅₋₁₀ aryl;
Y is —R¹⁸R¹⁹Si—O—, or —NR²¹CONR²²—;
Z is -SiR²⁵R²⁶R²⁷, -CR³¹R³²R³³ or C₅₋₁₀ aryl;
R¹, R², R³, R⁷, R⁸, R⁹, R¹¹, R¹², R¹⁸ R¹⁹, R²⁵, R²⁶, R²⁷, R³¹, R³² and R³³ are each independently hydrogen, C₁₋₂₀ alkyl, C₂₋₂₀ alkenyl, C₅₋₁₀ aryl, hydroxyl or C₁₋₂₀ alkoxyl;
R¹⁴, R¹⁵, R²¹, and R²² are each independently hydrogen, C₁₋₂₀ alkyl, C₂₋₂₀ alkenyl, C₅₋₁₀ aryl; and
s and t are each independently an integer from about 0 to about 6000, wherein the sum of s and t is not 0.

In one embodiment, V is absent, W is R¹R²R³SiO—; X is —R¹¹R¹²Si—O—; Y is —R¹⁸R¹⁹ Si—O—; Z is -SiR²⁵R²⁶R²⁷; and R¹, R², R³, R¹¹, R¹², R¹⁸, R¹⁹, R²⁵, R²⁶ and R²⁷ are each independently selected from C₁₋₂₀ alkyl (e.g., C₁ alkyl, such as methyl) or C₂₋₂₀ alkenyl (e.g., C₂ alkenyl, such as vinyl). In one embodiment, at least one of R¹, R², R³, R¹¹, R¹², R¹⁸, R¹⁹, R²⁵, R²⁶ and R²⁷ is C₂₋₂₀ alkenyl, for example, C₂ alkenyl (e.g., vinyl). In another embodiment, at least two of R¹, R², R³, R¹¹, R¹², R¹⁸, R¹⁹, R²⁵, R²⁶ and R²⁷ are C₂₋₂₀ alkenyl, for example, C₂ alkenyl (e.g., vinyl). In some embodiments, at least one of R¹, R², R³, R²⁵, R²⁶ and R²⁷ are each C₂₋₂₀ alkenyl, for example, C₂ alkenyl (e.g., vinyl).

In one embodiment, V is absent, W is R¹R²R³SiO—; X is —R¹¹R¹²Si—O—; Y is —R¹⁸R¹⁹ Si—O—; Z is -SiR²⁵R²⁶R²⁷; and R¹, R², R³, R²⁵, R²⁶ and R²⁷ are each independently selected from C₁₋₂₀ alkyl (e.g., C₁ alkyl, such as methyl) or C₂₋₂₀ alkenyl (e.g., C₂ alkenyl, such as vinyl); and R¹¹, R¹², R¹⁸, and R¹⁹ are each independently selected from C₁₋₂₀ alkyl (e.g., C₁ alkyl, such as methyl). In one embodiment, at least one of R¹, R², R³, and at least one of R²⁵, R²⁶ and R²⁷ is C₂₋₂₀ alkenyl, for example, C₂ alkenyl (e.g., vinyl). In one embodiment, one of R¹, R², R³ is C₂ alkenyl (e.g., vinyl) and the others are C₁₋₂₀ alkyl (e.g., C₁ alkyl, such as methyl), and at least one of R²⁵, R²⁶ and R²⁷ is C₂₋₂₀ alkenyl, for example, C₂ alkenyl (e.g., vinyl) and the others are C₁₋₂₀ alkyl (e.g., C₁ alkyl, such as methyl). In one embodiment, at least one of R¹¹ or R¹² and at least one of R¹⁸ or R¹⁹ is C₂₋₂₀ alkenyl, for example, C₂ alkenyl (e.g., vinyl) for at least one repeat unit. In one embodiment, one of R¹¹ or R¹² is C₂ alkenyl (e.g., vinyl) and the others are C₁₋₂₀ alkyl (e.g., C₁ alkyl, such as methyl), and at least one of R¹⁸ or R¹⁹ is C₂₋₂₀ alkenyl, for example, C₂ alkenyl (e.g., vinyl) and the others are C₁₋₂₀ alkyl (e.g., C₁ alkyl, such as methyl) for at least one repeat unit.

In some embodiments, the organopolysiloxane includes vinyl moieties only at the terminal caps of the polymer. In some embodiments, the organopolysiloxane is substantially vinyl functionalized. In some embodiments, the organopolysiloxane include vinyl moieties only in the repeat units, but not at the terminal cap of the polymer. In other embodiments, the organopolysiloxane includes vinyl moieties at both the terminal cap or in the repeat unit of the polymer. In one embodiment, the polymer includes two vinyl moieties located either at the terminal cap, or within the repeat unit, or a combination thereof. In at least one embodiment, the organopolysiloxane includes vinyl moieties only at the terminal caps of the polymer and contains Si-H units only within the repeat units and not at the terminal caps.

In one embodiment, on average at least two vinyl moieties are present in the polymer. In a specific embodiment, at least two vinyl moieties are present in the polymer and at least two vinyl moieties are present on the two terminal caps of the polymer. In a specific embodiment, only two vinyl moieties are present in the polymer. In a specific embodiment, only two vinyl moieties are present in the polymer and are located on each of the terminal caps. In a specific embodiment, on average at least two vinyl moieties are present in the polymer and at least two vinyl moieties are present in one or more repeat units of the polymer. In a specific embodiment, at least two vinyl moieties are present anywhere in the polymer, but separated from another vinyl moiety by about 2000 repeat units, for example, 1500, 1600, 1700, 1800, 1900, 2000, 2100, 2200, 2300, 2400, or 2500 repeat units. In a specific embodiment, on average at least two vinyl moieties are present anywhere in the polymer, but separated from another vinyl moiety by about 850 repeat units, for example, 350, 450, 550, 650, 750, 850, 950, 1050, 1150, 1250, or 1350 repeat units. In a specific embodiment, on average greater two vinyl moieties are present anywhere in the polymer, but separated from another vinyl moiety by about 40 repeat units, for example, 5, 10, 15, 20, 25, 30, 35, 40, 45, 50, 55, 60, 65, 70, 75, or 80 repeat units. In a specific embodiment, one or more Si-H units are present in addition to the vinyl moiety. Alternatively, in one embodiment, if a vinyl moiety is present then a Si—H is not present.

In one embodiment, V is absent, W is R′R2R3SiO—; X is —R¹¹R¹²Si—O—; Y is —R¹⁸R¹⁹ Si—O—; Z is -SiR²⁵R²⁶R²⁷; R¹, R², R³, R¹¹, R¹², R¹⁸, R¹⁹, R²⁵, R²⁶ and R²⁷ are each independently selected from hydrogen or C₁₋₂₀ alkyl (e.g., C₁ alkyl, such as methyl). In one embodiment, R¹, R², R³, R²⁵, R²⁶ and R²⁷ are each independently selected from C₁₋₂₀ alkyl (e.g., C₁ alkyl, such as methyl); and R¹¹, R¹², R¹⁸, and R¹⁹ are each independently selected from hydrogen or C₁₋₂₀ alkyl (e.g., C₁ alkyl, such as methyl), wherein at least one of R¹¹, R¹², R¹⁸, and R¹⁹ are hydrogen for at least one repeat unit. In one embodiment, on average greater than two Si-H units (e.g. one or more of R¹¹, R¹², R¹⁸, and R¹⁹ is hydrogen) are present in the polymer, for example 3- 15 Si-H units may be present. In a specific embodiment, 8 Si-H units are present on average. In one embodiment, one or more Si-H units (e.g. one or more of R¹¹, R¹², R¹⁸, and R¹⁹ is hydrogen) are present in the polymer. In one embodiment, at least two repeat units on average include a -Si-H unit (e.g. one or more of R¹¹, R¹², R¹⁸, and R¹⁹ is hydrogen). In one embodiment, at least three repeat units on average include a -Si-H unit (e.g. one or more of R¹¹, R¹², R¹⁸, and R¹⁹ is hydrogen). In one embodiment, at least four repeat units on average include a -Si-H unit (e.g. one or more of R¹¹, R¹², R¹⁸, and R¹⁹ is hydrogen). In one embodiment, at least five repeat units on average include a -Si-H unit (e.g. one or more of R¹¹, R¹², R¹⁸, and R¹⁹ is hydrogen). In one embodiment, at least six repeat units on average include a -Si-H unit (e.g. one or more of R¹¹, R¹², R¹⁸, and R¹⁹ is hydrogen). In one embodiment, at least seven repeat units on average include a -Si-H unit (e.g. one or more of R¹¹, R¹², R¹⁸, and R¹⁹ is hydrogen). In one embodiment, at least eight repeat units on average include a -Si-H unit (e.g. one or more of R¹¹, R¹², R¹⁸, and R¹⁹ is hydrogen). In one embodiment, a Si-H unit may be present in one or both the terminal caps in addition to being present in a repeat unit as described above. In one embodiment, one or more Si-H units may be present only in a repeat unit as described above, and not present in either of the terminal caps. In a specific embodiment, Si-(alkyl) or Si-(vinyl) units may also be present in the polymer. In a specific embodiment, only Si-CH₃ and Si-H units are present. In a specific embodiment, repeat units or terminal caps include C₁-C₂₀alkyl, specifically methyl groups, for the non-Si-H positions of the polymer.

In a specific embodiment, on average at least two Si-H units are present in the polymer. In a specific embodiment, on average at least two Si-H moieties are present anywhere in the polymer, but separated from another Si-H moiety by about 2000 repeat units, for example, 1500, 1600, 1700, 1800, 1900, 2000, 2100, 2200, 2300, 2400, or 2500 repeat units. In a specific embodiment, on average at least two Si-H moieties are present only in the repeat units of the polymer and not the terminal cap, and are separated from another Si-H moiety by about 2000 repeat units, for example, 1500, 1600, 1700, 1800, 1900, 2000, 2100, 2200, 2300, 2400, or 2500 repeat units. In a specific embodiment, on average at least two Si-H units are present anywhere in the polymer, but separated from another Si-H moiety by about 850 repeat units, for example, 350, 450, 550, 650, 750, 800, 850, 950, 1050, 1150, 1250, or 1350 repeat units. In a specific embodiment, on average at least two Si-H moieties are present only in the repeat units of the polymer and not the terminal caps, and are separated from another Si-H moiety by about 2000 repeat units, for example, 350, 450, 550, 650, 750, 800, 850, 950, 1050, 1150, 1250, or 1350 repeat units. In a specific embodiment, on average greater than two Si-H units are present anywhere in the polymer, but separated from another Si-H moiety by about 40 repeat units, for example, 5, 10, 15, 20, 25, 30, 35, 40, 45, 50, 55, 60, 65, 70, 75, or 80 repeat units. In a specific embodiment, on average at least two Si-H moieties are present only in the repeat units of the polymer and not the terminal caps, and are separated from another Si-H moiety by about 2000 repeat units, for example, 5, 10, 15, 20, 25, 30, 35, 40, 45, 50, 55, 60, 65, 70, 75, or 80 repeat units.

In one aspect of any one of the above embodiments, the sum of s and t is an integer from about 1000 to about 8000; from about 1300 to about 2700; from about 1500 to about 2700; from about 1600 to about 2600; from about 1600 to about 2500; from about 1700 to about 2500; from about 1800 to about 2400; from about 1800 to about 2300; from about 1900 to about 2300; from about 2000 to about 2200; from about 2050 to about 2150; from about 2100.

In one aspect of any one of the above embodiments, the sum of s and t is an integer from about 200 to about 1100; from about 600 to about 1100; from about 700 to about 1000; from about 800 to about 900; from about 825 to about 875; from about 850; from about 200 to about 800; from about 225 to about 700; from about 250 to about 600; from about 275 to about 500; from about 300 to about 400; from about 350 to about 400; from about 375. In a specific embodiment, the sum of s and t is an integer from about 850.

In one aspect of any one of the above embodiments, the sum of s and t is an integer from about 5 to about 1300; from about 10 to about 1100; from about 10 to about 600; from about 15 to about 500; from about 15 to about 400; from about 20 to about 300; from about 20 to about 200; from about 25 to about 100; from about 25 to about 75; from about 30 to about 50; from about 40.

In some embodiments, the composition includes compounds of formula II:

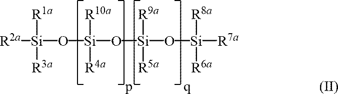

wherein R^{1a}, R^{2a}, R^{3a}, R^{4a}, R^{5a}, R^{6a}, R^{7a}, R^{8a}, R^{9a} and R^{10a} are each independently selected from hydrogen, C₁₋₂₀ alkyl, C₂₋₂₀ alkenyl, C₅₋₁₀ aryl, hydroxyl or C₁₋₂₀ alkoxyl and p and q are each independently an integer from between 10 and about 6000.

In some embodiments, the organopolysiloxane is a compound of formula IIa:

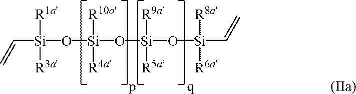

wherein R^{1a},’ R^{3a′}, R^{4a′}, R^{5a′}, R^{6a′}, R^{8a′}, R^{9a′} and R^{10a′} are each independently selected from hydrogen, C₁₋₂₀ alkyl, C₂₋₂₀ alkenyl, C₅₋₁₀ aryl, hydroxyl or C₁₋₂₀ alkoxyl and p and q are each independently an integer from between 10 and about 6000. In one embodiment, R^{1a}, R^{3a′}, R^{4a′}, R^{5a′}, R^{6a′}, R^{8a′}, R^{9a′} and R^{10a′} are alkyl (e.g., C₁ alkyl, such as methyl).

In some embodiments, the organopolysiloxane is vinyl functionalized. In some embodiments, the organopolysiloxane is substantially vinyl functionalized. The language “vinyl functionalized organopolysiloxane” includes organopolysiloxanes that have at least one vinyl group at both terminal ends of the polymer. Specifically, the language “vinyl functionalized organopolysiloxane” includes organopolysiloxanes of formula II1 in which one or both of R^{2a} and R^{7a} are substituted with a C₂ alkyl moiety, for example, a vinyl moiety (e.g., —CH═CH₂). In a specific embodiment, a “vinyl functionalized organopolysiloxane” includes organopolysiloxanes of formula II1 in which one or both of R^{2a} and R^{7a} are substituted with a C₂ alkyl moiety, for example, a vinyl moiety (e.g., —CH═CH₂), and R^{1a}, R^{3a}, R^{4a}, R^{5a}, R^{6a}, R^{8a}, R^{9a} and R^{10a} are independently selected from C₁₋₂₀ alkyl, for example, methyl.

In some embodiments, the organopolysiloxane is a compound of formula IIb:

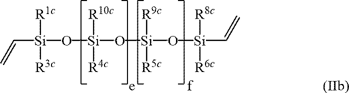

wherein R^{1c}, R^{3c}, R^{4c}, R^{5c}, R^{6c}, R^{8c}, R^{9c} and R^{10c} are each independently selected from hydrogen, C₁₋₂₀ alkyl, C₂₋₂₀ alkenyl, C₅₋₁₀ aryl, hydroxyl or C₁₋₂₀ alkoxyl and e and f are each independently an integer from between 10 and about 6000. In one embodiment, R ^{1c}, R^{3c}, R^{4c}, R^{5c}, R^{6c}, R^{8c}, R^{9c} and R^{10c} are alkyl (e.g., C₁ alkyl, such as methyl). In some embodiments, the sum of e and f is an integer from about 1000 to about 8000; from about 1300 to about 2700; from about 1500 to about 2700; from about 1600 to about 2600; from about 1600 to about 2500; from about 1700 to about 2500; from about 1800 to about 2400; from about 1800 to about 2300; from about 1900 to about 2300; from about 2000 to about 2200; from about 2050 to about 2150; from about 2100.

In some embodiments, the organopolysiloxane is a compound of formula IIc:

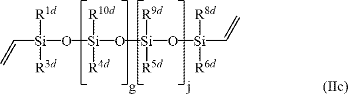

wherein R^{1d}, R^{3d}, R^{4d}, R^{5d}, R^{6d}, R^{8d}, R^{9d} and R^{10d} are each independently selected from hydrogen, C₁₋₂₀ alkyl, C₂₋₂₀ alkenyl, C₅₋₁₀ aryl, hydroxyl or C₁₋₂₀ alkoxyl and g and j are each independently an integer from between 10 and about 6000. In one embodiment, R ^{1d}, R^{3d}, R^{4d}, R^{5d}, R^{6d}, R^{8d}, R^{9d} and R^{10d} are alkyl (e.g., C₁ alkyl, such as methyl). In some embodiments, the sum of g and j is an integer from about 200 to about 1100; from about 600 to about 1100; from about 700 to about 1000; from about 800 to about 900; from about 825 to about 875; from about 850; from about 200 to about 800; from about 225 to about 700; from about 250 to about 600; from about 275 to about 500; from about 300 to about 400; from about 350 to about 400; from about 375. In some embodiments, the sum of g and j is an integer from about 850.

In some embodiments, the organopolysiloxane is an alkenyl-functionalized organopolysiloxane. In one embodiment, the alkenyl-functionalized polymer includes one or more alkenyl-functionalized side chains. In this embodiment, any of R₁, R₂, R₃, R₄, R₅ and R₆ may independently be the fragment:

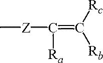

wherein Z is as defined above for Z₁ and Z₂ and Rₐ, R_{b}, and R_{c} are independently selected from hydrogen, substituted or unsubstituted branched or straight chain C₁-C₁₀ alkyl, alkenyl, or alkynyl group, including without limitation methyl, ethyl, propyl, isopropyl, butyl, isobutyl, t-butyl, pentyl, hexyl, vinyl, allyl, butenyl, pentenyl, hexenyl, propynyl, butynyl, n-pentyl, iso-pentyl, neo-pentyl, tert-pentyl; cycloalkyl, heterocycloalkyl, haloalkyl, benzyl, alkyl-aryl; substituted or unsubstituted aryl or heteroaryl groups; C₁-C₆ alkoxy, amino, alkyl amino, dialkyl amino, hydroxyl, carboxy, cyano, or halogen. Preferably R₄ is methyl. Exemplary alkenyl-functionalized organopolysiloxanes include without limitation methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl-(3,3,-trifluoropropyl)siloxane copolymers.

In one embodiment, provided herein is a composition including a curable silicone formulation containing: components (a), (d) and at least one of (b) or (c):
a. a polyorganosiloxane resin, composed of M and Q units having at least 3 alkenyl groups per molecule, herein after called “SiVi” groups,
b. a polyorganosiloxane compound having at least 2 Si-bonded hydrogen groups on the polysiloxane chain, hereinafter called “SiH” groups,
c. a telechelic polyorganosiloxane compound having terminal Si-H groups, and
d. a hydrosilylation catalyst for the reaction of SiH groups with SiVi groups,
e. a liquid diluent in an amount of from 0% to maximum 90% by weight of the composition said components reacting together by hydrosilylation at a temperature below 40° C. when they cure to form a continuous film on the substrate.

In one embodiment, a formulation meeting these requirements is able to cure quickly at room temperature/ambient as a film on a substrate and can provide good balance between adhesion and tackiness requirements; the film can show good adhesion to the substrate while the surface opposite to the substrate shows low tack.

In one embodiment, the organopolysiloxane is a polydiorganosiloxane resin having at least 3 silicon-bonded alkenyl groups per molecule, with preferably the remaining silicon-bonded organic groups being selected from alkyl and aryl groups, said polydiorganosiloxane resin preferably has a molecular weight from 1,500 daltons to 50,000 daltons.

Suitable polyorganosiloxane resins having silicon bonded unsaturated groups (a) are those with sufficient unsaturated groups for formation of the polymer network. The functional siloxane resin structure may include R₃SiO_{½} units (M units) and SiO_{4/2} units (Q units) wherein each R is independently a linear, branched or cyclic hydrocarbon group having 1-20 carbon atoms. Each R can be identical or different, as desired. The hydrocarbon group of R can be exemplified by alkyl groups such as methyl, ethyl, propyl, butyl, hexyl, octyl, vinyl, hexenyl and aryl groups such as phenyl.

### 1.3 Hydride Functionalized Polysiloxane

In some embodiments, the composition includes at least one hydride functionalized polysiloxane. The language “hydride functionalized polysiloxane” includes compounds of formula III:

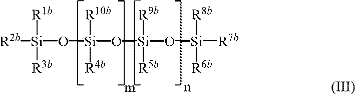

wherein R^{1b}, R^{2b}, R^{3b}, R^{4b}, R^{5b}, R^{6b}, R^{7b}, R^{8b}, R^{9b} and R^{10b} are each independently selected from hydrogen, C₁₋₂₀ alkyl, C₂₋₂₀ alkenyl, C₅₋₁₀ aryl, hydroxyl or C₁₋₂₀ alkoxy and m and n are each independently an integer from between 10 and about 6000, provided that at least one of R^{1b}, R^{2b}, R^{3b}, R^{4b}, R^{5b}, R^{6b}, R^{7b}, R^{8b}, R^{9b} and R^{10b} is hydrogen. In some embodiments, at least one of R^{1b}, R^{2b}, R^{3b}, R^{4b}, R^{5b}, R^{6b}, R^{7b}, R^{8b}, R^{9b} and R^{10b} is hydrogen and the remainder are C₁₋₂₀ alkyl. In some embodiments, at least two of R^{1b}, R^{2b}, R^{3b}, R^{4b}, R^{5b}, R^{6b}, R^{7b}, R^{8b}, R^{9b} and R^{10b} are hydrogen (e.g., two Si-H units per functionalized hydride polysiloxane molecule). In other embodiments, at least three of R^{1b}, R^{2b}, R^{3b}, R^{4b}, R^{5b}, R^{6b}, R^{7b}, R^{8b}, R^{9b} and R^{10b} are hydrogen (e.g., three Si-H units per functionalized hydride polysiloxane molecule). In some embodiments, at least two of R^{1b}, R^{2b}, R^{3b}, R^{4b}, R^{5b}, R^{6b}, R^{7b}, R^{8b}, R^{9b} and R^{10b} are hydrogen (e.g., two Si-H units per functionalized hydride polysiloxane molecule) and the remainder are C₁₋₂₀ alkyl. In other embodiments, at least three of R^{1b}, R^{2b}, R^{3b}, R^{4b}, R^{5b}, R^{6b}, R^{7b}, R^{8b}, R^{9b} and R^{10b} are hydrogen (e.g., three Si-H units per functionalized hydride polysiloxane molecule) and the remainder are C₁₋₂₀ alkyl. In some embodiments, at least two of R^{4b}, R^{5b}, R^{9b} and R^{10b} are hydrogen (e.g., two Si-H units per functionalized hydride polysiloxane molecule) and the remainder are C₁₋₂₀ alkyl. In other embodiments, at least three of R^{4b}, R^{5b}, R^{9b} and R^{10b} are hydrogen (e.g., three Si-H units per functionalized hydride polysiloxane molecule) and the remainder are C₁₋₂₀ alkyl. In some embodiments, at least two of R^{4b}, R^{5b}, R^{9b} and R^{10b} are hydrogen (e.g., two Si-H units per functionalized hydride polysiloxane molecule) and the remainder and R^{1b}, R^{2b}, R^{3b}, R^{6b}, R^{7b}, and R^{8b} are C₁₋₂₀ alkyl. In other embodiments, at least three of R^{4b}, R^{5b}, R^{9b} and R^{10b} are hydrogen (e.g., three Si-H units per functionalized hydride polysiloxane molecule) and the remainder and R^{1b}, R^{2b}, R^{3b}, R^{6b}, R^{7b}, and R^{8b} are C₁₋₂₀ alkyl.

In one embodiment, at least greater than two repeat units of formula III include a -Si-H unit (e.g. one or more of R^{4b}, R^{5b}, R^{9b} and R^{10b} is hydrogen). In one embodiment, at least greater than two repeat units of formula III include a -Si-H unit (e.g. one or more of R^{4b}, R^{5b}, R^{9b} and R^{10b} is hydrogen) and the remaining non-Si-H repeat units are Si-CH₃. For example, on average 2 to 15 repeat units of formula III include a Si-H unit. In one embodiment, at least two repeat units of formula III include a -Si-H unit (e.g. one or more of R^{4b}, R^{5b}, R^{9b} and R^{10b} is hydrogen). In one embodiment, at least three repeat units of formula III include a -Si-H unit (e.g. one or more of R^{4b}, R^{5b}, R^{9b} and R ^{10b} is hydrogen). In one embodiment, at least four repeat units of formula III include a -Si-H unit (e.g. one or more of R^{4b}, R^{5b}, R^{9b} and R^{10b} is hydrogen). In one embodiment, at least five repeat units of formula III include a -Si-H unit (e.g. one or more of R^{4b}, R^{5b} , R^{9b} and R^{10b} is hydrogen). In one embodiment, at least six repeat units of formula III include a -Si-H unit (e.g. one or more of R^{4b}, R^{5b}, R^{9b} and R^{10b} is hydrogen). In one embodiment, at least seven repeat units of formula III include a -Si-H unit (e.g. one or more of R^{4b}, R^{5b}, R^{9b} and R^{10b} is hydrogen). In one embodiment, at least eight repeat units of formula III include a -Si-H unit (e.g. one or more of R^{4b}, R^{5b}, R^{9b} and R^{10b} is hydrogen). In a specific embodiment, the non Si-H positions may include a Si-(alkyl) or Si-(vinyl) unit. In a specific embodiment, the non-Si-H positions are Si-CH₃. In some of the embodiments, R^{1b}, R^{2b}, R^{3b}, R^{6b}, R^{7b}, and R^{8b} are C₁₋₂₀ alkyl. In a specific embodiment, the Si-H positions are not present in the terminal caps. In some embodiments, the compound of formula III is substantially alkyl-terminated. In some embodiments, the compound of formula III is alkyl-terminated. In one embodiment, the Si-H units in the hydride-functionalized organopolysiloxanes are separated by 5, 10, 15, 20, 25, 30, 35, 40, 45, 50, 60, 65, 70, 75, 80, 85, 90, 100, 125, 150, or 200 repeat units.

In one aspect of any one of the above embodiments, the sum of m and n is an integer from about 10 to about 1300; from about 10 to about 1100; from about 10 to about 600; from about 15 to about 500; from about 15 to about 400; from about 20 to about 300; from about 20 to about 200; from about 25 to about 100; from about 25 to about 75; from about 30 to about 50; from about 40.

In some embodiments, the hydride functionalized polysiloxane includes Si-H units only at the terminal caps of the polymer. In some embodiments, the polysiloxane include Si-H units only in the repeat units, but not at the terminal caps of the polymer. In other embodiments, the polysiloxane includes Si-H units at both the terminal cap or in the repeat unit of the polymer. In one embodiment, the polysiloxane includes two to twelve Si-H units on average located either at the terminal cap, or within the repeat unit, or a combination thereof. In one embodiment, the polysiloxane includes four to fifteen Si-H units on average located either at the terminal cap, or within the repeat unit, or a combination thereof. In one embodiment, the polysiloxane includes eight Si-H units on average located either at the terminal cap, or within the repeat unit, or a combination thereof. In one embodiment, the polysiloxane includes two to twelve Si-H units on average located within the repeat unit, and not at the terminal caps. In one embodiment, the polysiloxane includes four to fifteen Si-H units on average located within the repeat unit, and not at the terminal caps. In one embodiment, the polysiloxane includes eight Si-H units on average located within the repeat unit, and not at the terminal caps. In some embodiments, the hydride functionalized polysiloxane is substantially alkyl terminated.

In other embodiments, the hydride functionalized polysiloxane is alkyl terminated. In other embodiments, the hydride functionalized polysiloxane is substantially alkyl terminated. The language “alkyl terminated” includes hydride functionalized polysiloxanes of formula III in which one or both of R^{2b} and R^{7b} are C₁₋₂₀ alkyl. In some embodiments, “alkyl terminated” includes hydride functionalized polysiloxanes of formula III in which one, two, three, four, five or six of R^{1b}, R^{2b}, R^{3b}, R^{6b}, R^{7b} and R^{8b} are C₁₋₂₀ alkyl. In one embodiment, R^{1b}, R^{2b}, R^{3b}, R^{4b}, R^{5b}, R^{6b}, R^{7b}, R^{8b} and R^{10b} are each C₁₋₂₀ alkyl, for example, C₁ alkyl (e.g., methyl) and R^{9b} is hydrogen. In one embodiment, R^{1b}, R^{2b}, R^{3b}, R^{4b}, R^{5b}, R^{6b}, R^{7b}, R^{8b} and R^{9b} are each C₁₋₂₀ alkyl, for example, C₁ alkyl (e.g., methyl) and R^{10b} is hydrogen.

In certain embodiments, the organopolysiloxane having carbon double bonds has a weight percent of carbon double bond-containing repeat units of between about 0.01 and about 2%, and preferably, between about 0.03 and about 0.6%. In certain embodiments, the organopolysiloxane having carbon double bonds has a vinyl equivalent per kilogram of between about 0.005 and about 0.5, and preferably, between about 0.01 and about 0.25. An approximate molar amount of the carbon double bonds in the organopolysiloxane can be calculated based on the average molecular weight of the organopolysiloxane.

In certain embodiments, the vinyl functionalized organopolysiloxane has a viscosity above about 100 cP and below about 1,000,000 cP at about 25° C. In certain embodiments, the vinyl functionalized organopolysiloxane has a viscosity below about 750,000 cP, below about 500,000 cP, or below about 250,000 cP at about 25° C. In preferred embodiments, the vinyl functionalized organopolysiloxane has a viscosity below about 200,000 cP, below about 175,000 cP, below about 150,000 cP, below about 125,000 cP, below about 100,000 cP, or below about 80,000 cP at about 25° C. In certain embodiments, the vinyl functionalized organopolysiloxane has a viscosity above about 100 cP, above about 500 cP, or above about 1000 cP at about 25° C. In preferred embodiments, the vinyl functionalized organopolysiloxane has a viscosity above about 2000 cP, above about 5000 cP, above about 7500 cP, or above about 10,000 cP at about 25° C. In further preferred embodiments, the vinyl functionalized organopolysiloxane has a viscosity above about 15,000 cP at about 25° C.

In certain embodiments, the vinyl functionalized organopolysiloxane has a viscosity between about 10,000 and about 2,000,000 cSt at about 25° C. In preferred embodiments, the vinyl functionalized organopolysiloxane has a viscosity above about 20,000, above about 40,000, above about 60,000, above about 80,000, or above about 100,000 cSt at about 25° C. In further preferred embodiments, the vinyl functionalized organopolysiloxane has a viscosity above about 125,000 or above about 150,000 cSt at about 25° C. In preferred embodiments, the vinyl functionalized organopolysiloxane has a viscosity below about 1,000,000 cSt, below about 500,000 cSt, below about 450,000, below about 400,000, below about 350,000, below about 300,000, or below about 250,000 cSt at about 25° C. In further preferred embodiments, the vinyl functionalized organopolysiloxane has a viscosity below about 200,000 or below about 180,000 cSt at about 25° C. In further preferred embodiments, the vinyl functionalized organopolysiloxane has a viscosity of about 165,000 cSt at about 25° C.

In one embodiment, the vinyl functionalized organopolysiloxane has a viscosity between about 500 and about 500,000 cSt or cP at about 25° C. In one embodiment, the vinyl functionalized organopolysiloxane has a viscosity between about 150,000 and about 185,000 cSt or cP at about 25° C. In one embodiment, the vinyl functionalized organopolysiloxane has a viscosity of about 165,000 cSt or cP at about 25° C. In one embodiment, the vinyl functionalized organopolysiloxane has a viscosity of about 10,000 cSt or cP at about 25° C.

In one embodiment, the vinyl functionalized organopolysiloxane has a viscosity between about 150,000 and about 185,000 cSt or cP at about 25° C., and the hydride functionalized polysiloxane has a viscosity of between about 30 and about 100 cSt or cP at about 25° C. In one embodiment, the vinyl functionalized organopolysiloxane has a viscosity of about 165,000 cSt or cP at about 25° C., and the hydride functionalized polysiloxane has a viscosity of about 45 cSt or cP at about 25° C. In one embodiment, the vinyl functionalized organopolysiloxane has a viscosity of about 165,000 cSt or cP at about 25° C., and the hydride functionalized polysiloxane has a viscosity of about 50 cSt or cP at about 25° C.

In certain embodiments, the vinyl functionalized organopolysiloxane has an average molecular weight between about 60,000 Da and about 500,000 Da. In preferred embodiments, the vinyl functionalized organopolysiloxane has an average molecular weight above about 72,000 Da, about 84,000 Da, about 96,000 Da, or about 100,000 Da. In further preferred embodiments, the vinyl functionalized organopolysiloxane has an average molecular weight above about 140,000 Da, or about 150,000 Da. In preferred embodiments, the vinyl functionalized organopolysiloxane has an average molecular weight below about 200,000 Da, below about 190,000 Da, about 180,000 Da, or about 170,000 Da. In further preferred embodiments, the vinyl functionalized organopolysiloxane has an average molecular weight below about 160,000 Da. In further preferred embodiments, the vinyl functionalized organopolysiloxane has an average molecular weight of about 155,000 Da.

In certain embodiments, the vinyl functionalized organopolysiloxane has an average molecular weight between about 400 and about 500,000 Da. In preferred embodiments, the vinyl functionalized organopolysiloxane has an average molecular weight above about 500 Da, about 800 Da, about 1,200 Da, or about 1,800 Da. In further preferred embodiments, the vinyl functionalized organopolysiloxane has an average molecular weight above about 2,000 Da. In preferred embodiments, the vinyl functionalized organopolysiloxane has an average molecular weight below about 250,000 Da, below about 140,000 Da, below about 100,000 Da, below about 72,000 Da, below about 62,700 Da, below about 49,500 Da, below about 36,000 Da, or below about 28,000 Da. In further preferred embodiments, the vinyl functionalized organopolysiloxane has an average molecular weight below about 17,200 Da. In further preferred embodiments, the vinyl functionalized organopolysiloxane has an average molecular weight between about 2,200 Da and 6,000 Da.

In certain embodiments, the molar ratio of Si-H functional group to alkenyl (e.g., vinyl) functional group is from about 100:1 to about 1:5. In preferred embodiments, the molar ratio of Si-H functional group to alkenyl-functional group from is about 45:1 to about 15:1. In certain embodiments, the molar ratio of Si-H functional group to alkenyl-functional group is from about 100:1 to about 1:5. In preferred embodiments, the molar ratio of Si-H functional group to alkenyl-functional group from is about 45:1 to about 15:1. In certain embodiments, the Si-H to alkenyl molar ratio of the polymers in the composition is about 1:5 to about 100:1; about 10:1 to about 30:1; or about 20:1 to about 25:1. In certain embodiments, the molar ratio of Si-H functional group to alkenyl-functional group from is about 10:1 to about 100:1. In preferred embodiments, the molar ratio of Si-H functional group to alkenyl-functional group from is about 30:1 to about 60:1. In preferred embodiments, the molar ratio of Si-H functional group to alkenyl-functional group from is about 20:1 to about 50:1.

In one embodiment, the vinyl functionalized organopolysiloxane is vinyl terminated. In preferred embodiments, the vinyl functionalized organopolysiloxane is selected from vinyl terminated polydimethylsiloxane, vinyl terminated diphenylsiloxane-dimethylsiloxane copolymers, vinyl terminated polyphenylmethylsiloxane, vinylphenylmethyl terminated vinylphenylsiloxane-phenylmethylsiloxane copolymer, vinyl terminated trifluoropropylmethylsiloxane-dimethylsiloxane copolymer, vinyl terminated diethylsiloxane-dimethylsiloxane copolymer, vinylmethylsiloxane-dimethylsiloxane copolymer, trimethylsiloxy terminated, vinylmethylsiloxane-dimethylsiloxane copolymers, silanol terminated, vinylmethylsiloxane-dimethylsiloxane copolymers, vinyl terminated, vinyl gums, vinylmethylsiloxane homopolymers, vinyl T-structure polymers, vinyl Q-structure polymers, unsaturated organopolymers (non-limiting examples of which include one or more of unsaturated fatty alcohols, unsaturated fatty acids, unsaturated fatty esters, unsaturated fatty amide, unsaturated fatty urethane, unsaturated fatty urea, ceramide, cocetin, lecithin and sphingosine), monovinyl terminated polydimethylsiloxanes, vinylmethylsiloxane terpolymers, vinyl-methoxysilane homopolymers, vinyl terminated polyalkylsiloxane polymers, vinyl terminated polyalkoxysiloxane polymers and combinations thereof. In further preferred embodiments, the vinyl functionalized organopolysiloxane is vinyl dimethicone.

In a preferred embodiment, the Si-H units in the hydride functionalized polysiloxane are spaced on average by at least about 1 repeat units, about 2 repeat units, about 5 repeat units, about 10 repeat units, about 20 repeat units, about 40 repeat units, about 200 repeat units, about 400 repeat units, about 1,000 repeat units, or about 2,000 repeat units.

In certain embodiments, the hydride functionalized polysiloxane has a viscosity between about 2 to about 500,000 cSt at about 25° C. In preferred embodiments, the hydride functionalized polysiloxane has a viscosity above about 3 cSt, above about 4 cSt, or above about 12 cSt at about 25° C. In further preferred embodiments, the hydride functionalized polysiloxane has a viscosity above about 40 cSt at about 25° C. In preferred embodiments, the hydride functionalized polysiloxane has a viscosity below about 200,000, below about 100,000, below about 50,000, below about 20,000, below about 10,000, below about 5,000, below about 2,000, or below about 1,000 cSt at about 25° C. In further preferred embodiments, the hydride functionalized polysiloxane has a viscosity below about 500 cSt at about 25° C. In further preferred embodiments, the hydride functionalized polysiloxane has a viscosity between about 45 to about 100 cSt at about 25° C.

In certain embodiments, the hydride functionalized polysiloxane having Si-H units includes such Si-H units at terminal units of the polymer, in non-terminal repeat units of the polymer, or a combination thereof. In preferred embodiments, the hydride functionalized polysiloxane having Si-H units includes such Si-H units in non-terminal repeat units of the polymer. In preferred embodiments, the Si-H-containing repeat units in the hydride functionalized polysiloxane are spaced on average by at least about 1 repeat units, about 2 repeat units, about 5 repeat units, about 10 repeat units, about 20 repeat units, about 40 repeat units, about 200 repeat units, about 400 repeat units, about 1,000 repeat units, or about 2,000 repeat units.

In certain embodiments, the hydride functionalized polysiloxane having Si-H units has a weight percent of Si-H-containing repeat units of between about 0.003% and about 100%, about 0.008% and about 50%, and preferably, between about 0.01% and about 25%. In certain embodiments, the hydride functionalized polysiloxane having Si-H units has an Si-H content of between about 0.1 mmol/g and about 20 mmol/g, about 0.5 mmol/g and about 10 mmol/g, and preferably, between about 1 mmol/g and about 5 mmol/g. An approximate molar amount of the Si-H units in the hydride functionalized polysiloxane can be calculated based on the average molecular weight of the organopolysiloxane. Average molecular weight, or molar mass, of the ingredients disclosed herein are commonly provided by the supplier of the ingredients, expressed in units of Dalton (Da) or its equivalent g/mol.

In preferred embodiments, the hydride functionalized polysiloxane is selected from hydride terminated polydimethylsiloxane, hydride terminated polyphenyl-(dimethylhydrosiloxy)siloxane, hydride terminated methylhydrosiloxane-phenylmethylsiloxane copolymer, trimethylsiloxy terminated methylhydrosiloxane-dimethylsiloxane copolymers, polymethylhydrosiloxanes, trimethylsiloxy terminated, polyethylhydrosiloxane, triethylsiloxane, methylhydrosiloxane-phenyloctylmethylsiloxane copolymer, methylhydrosiloxane-phenyloctylmethylsiloxane terpolymer, and combinations thereof. In further preferred embodiments, the hydride functionalized polysiloxane is hydrogen dimethicone.

Exemplary hydride functionalized polysiloxanes include without limitation alkyltrihydrosilanes, aryltrihydro-silanes, dialkyldihydrosilanes, diaryidihydrosilanes, trialkylhydrosilanes, triarylhydrosilanes, alkylhydrosiloxanes and arylhydrosiloxanes. Special mention may be made of polymethylhydrosiloxanes, t-butyldimethylhydrosilane, triethylhydrosilane, diethyldihydrosilane, triisopropylhydrosilane and mixtures thereof.

In some embodiments, the hydride functionalized polysiloxane is a hydrosilicon compound having at least 2 silicon-bonded hydrogen atoms per molecule, which preferably consists essentially of RHSiO— groups, R₂ZSiO— groups and optionally R₂ SiO— groups and preferably has a viscosity at about 25° C. of no more than 1,000 mm²/s, wherein R denotes an alkyl or aryl group having no more than 8 carbon atoms, and Z denotes H or R.

In certain embodiments, the organosiloxane polymers can be prepared according to the methods described in the disclosures of U.S. Pat. Nos. 8,691,202, 9,114,096, 9,308,221, 9,333,223, 9,724,363, 9,937,200 and 10,022,396 and International Patent Publication No. WO 2017/083398, the disclosures of which are incorporated herein by reference in their entireties. The siloxane polymers can be also prepared according to other methods apparent to those of skill in the art.

### 1.4 Bifunctional Organopolysiloxane Polymer

In certain embodiments, the bifunctional organopolysiloxane polymer is a linear siloxane polymer. In certain embodiments, the bifunctional organopolysiloxane polymer is a branched siloxane polymer.

In certain embodiments, the unsaturated group is not particularly limited, and may be, for example, a vinyl, styryl, allyl, methallyl, hexenyl, octenyl or alkynyl group. The preferred unsaturated group is a vinyl group.

In certain embodiments, the unsaturated group or the hydride group are terminal groups.

In certain embodiments, the siloxane polymer has a degree of polymerization of at least 20 and a dispersity index less than about 1.2, and wherein a ratio of unsaturated terminal groups to hydride terminal groups is substantially 1:1. In certain embodiments, the degree of polymerization is about 20 to about 200. In certain embodiments, the unsaturated terminal group is selected from the group consisting of vinyl, styryl, allyl, methallyl, hexenyl, octenyl and alkynyl. In certain embodiments, the siloxane backbone is selected from the group consisting of diphenylsiloxane, phenylmethylsiloxane, trifluoropropylmethylsiloxane, dimethylsilylethylsiloxane, and alkylmethylsiloxane. In certain embodiments, the siloxane backbone is dimethylsiloxane and the unsaturated group is vinyl. In certain embodiments, the film has no apparent crosslinking. In certain embodiments, the linear siloxane polymer is a monovinyl-monohydride terminated polysiloxane. In certain embodiments, the film is formed via hydrosilylation step-growth polymerization of the linear siloxane polymer. In certain embodiments, the linear siloxane polymer is capable of being reacted with a metal catalyst to form the film over the subject’s skin. In certain embodiments, the linear siloxane polymer is compounded with a reinforcing constituent prior to the reaction with the metal catalyst. In certain embodiments, the metal catalyst is a platinum catalyst. In certain embodiments, the reinforcing constituent is fumed silica. In certain embodiments, the linear siloxane polymer is a monovinyl-monohydride terminated polydimethylsiloxane.

In certain embodiments, the bifunctional organopolysiloxane polymer includes an organopolysiloxane with at least two alkenyl functional groups or at least one alkynyl functional group, either at the terminal or at the side chain or both, as shown in formula (I):

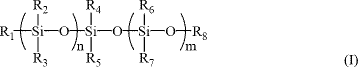

wherein at least two of R₁, R₄, R₅ and R₈ are alkenyl groups or at least one of R₁, R₄,
R₅ and R₈ is alkynyl group;
the rest of R₁, R₄, R₅ and R₈ are C₁₋₂₀ alkyl groups;
R₂, R₃, R₆ and R₇ are C₁₋₂₀ alkyl groups; and
the sum of n and m is between 2 to 1000,000.

In certain embodiments, the bifunctional organopolysiloxane polymer includes an organopolysiloxane with at least two hydride functional groups, either at the terminal or at the side chain or both, as shown in formula (II):

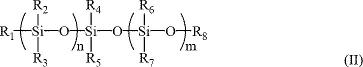

wherein at least two of R₁, R₄, R₅ and R₈ are hydride groups;
the rest of R₁, R₄, R₅ and R₈ are C₁₋₂₀ alkyl groups;
R₂, R₃, R₆ and R₇ are C₁₋₂₀ alkyl groups; and
the sum of n and m is between 2 to 1000,000.

In certain embodiments, the bifunctional organopolysiloxane polymer includes an organopolysiloxane with at least one alkenyl functional group or at least one alkynyl functional group; and at least one hydride functional group, either at the terminal or at the side chain or both, as shown in formula (III):

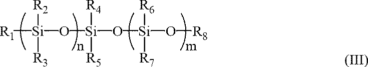

wherein at least one of R₁, R₄, R₅ and R₈ is an alkenyl functional group or at least one of R₁, R₄, R₅ and R₈ is alkynyl group; and at least one of R₁, R₄, R₅ and R₈ is a hydride group;
the rest of R₁, R₄, R₅ and R₈ are C₁₋₂₀ alkyl groups;
R₂, R₃, R₆ and R₇ are C₁₋₂₀ alkyl groups; and
the sum of n and m is between 2 to 1000,000.

In certain embodiments, the bifunctional organopolysiloxane polymer has a viscosity above about 1 cP, above about 10 cP, above about 100 cP, above about 500 cP, or above about 1000 cP at about 25° C. In preferred embodiments, the first part has a viscosity above about 2000 cP, above about 5000 cP at about 25° C. In further preferred embodiments, the first part has a viscosity above about 15,000 cP at about 25° C. In certain embodiments, the first part has a viscosity below about 1,000,000 cP, below about 750,000 cP, below about 500,000 cP, or below about 250,000 cP at about 25° C. In preferred embodiments, the first part has a viscosity below about 200,000 cP, below about 175,000 cP, below about 150,000 cP, below about 125,000 cP, below about 100,000 cP, or below about 50,000 cP, or below 25,000 cP at about 25° C.

In certain embodiments, the second part has a viscosity above about 1 cP, above about 10 cP, above about 100 cP, above about 500 cP, or above about 1000 cP at about 25° C. In preferred embodiments, the first part has a viscosity above about 2000 cP, above about 5000 cP at about 25° C. In further preferred embodiments, the first part has a viscosity above about 15,000 cP at about 25° C. In certain embodiments, the first part has a viscosity below about 1,000 ,000 cP, below about 750,000 cP, below about 500,000 cP, or below about 250,000 cP at about 25° C. In preferred embodiments, the first part has a viscosity below about 200,000 cP, below about 175,000 cP, below about 150,000 cP, below about 125,000 cP, below about 100,000 cP, or below about 50,000 cP, or below 25,000 cP at about 25° C.

In certain embodiments, the composition that forms the layer has a glass transition temperature below about 37° C. In preferred embodiments, the composition that forms the layer has a glass transition temperature below about 25° C. In further preferred embodiments, the composition that forms the layer has a glass transition temperature below about 0° C. In certain embodiments, the first part of the composition that forms the layer has a glass transition temperature below about 37° C. In preferred embodiments, the first part of the composition that forms the layer has a glass transition temperature below about 25° C. In further preferred embodiments, the first part of the composition that forms the layer has a glass transition temperature below about 0° C. In certain embodiments, the second part of the composition that forms the layer has a glass transition temperature below about 37° C. In preferred embodiments, the second part of the composition that forms the layer has a glass transition temperature below about 25° C. In further preferred embodiments, the second part of the composition that forms the layer has a glass transition temperature below about 0° C.

Another aspect provided herein is directed to a composition that forms a layer that does not significantly change the shine and/or gloss of the area over which the composition is applied. Shine and/or gloss can be measured on a specimen formed from the composition in vitro, for example, using a Glossmeter pursuant to the ASTM D523 Specular Gloss test, at 20°, 60°, and/or 85° measurement angels. The light and measurement angel can be selected based on the anticipated gloss range. For example, if the measurement made at 60° is greater than about 70 gloss units (GU), the measurement angle should be changed to 20° to optimize measurement accuracy. Conversely, if the measurement made at 60° is less than about 10 GU, the measurement angle should be changed to 85° to optimize measurement accuracy. 45° or 75° measurement angle may also be used depending on the gloss of the substrate used for the test. Various materials can be used as substrate to mimic normal, healthy skin for the test, for example, Cowhide Tooling leather in natural color. Shine and/or gloss change is indicated by the percentage increase or decrease of gloss units in a measurement area after the treatment comparing to before treatment. In certain embodiments, the shine and/or gloss change of the area treated with the composition is less than about 20%. In preferred embodiments, the shine and/or gloss change of the area treated with the composition is less than about 10%. In further preferred embodiments, the shine and/or gloss change of the area treated with the composition is less than about 5%.

Another aspect provided herein is directed to a composition that forms a layer that is resistant to environmental factors, such as exposure to heat, cold, wind, water, humidity, bodily fluids (e.g., blood, pus/liquor puris, urine, saliva, sputum, tears, semen, milk, or vaginal secretion), sebum, saline, seawater, soapy water, detergent water, or chlorinated water. Such resistance to environmental factors is represented by the minimal weight increase upon exposures to these environmental factors. The weight change of the layer is determined by using the ASTM D2765-95 Determination of Gel Content and Swell Ratio of Crosslinked Ethylene Plastics test using a weight scale. In certain embodiments, the weight of the layer increases by less than about 10% upon exposure to such environmental factors at about 1-hour time point (i.e., 1 hour after application of the composition disclosed herein), about 4-hour, about 6-hour, about 12-hour, about 24-hour, about 30-hour, about 36-hour, about 48-hour, or between 48 hours and one week time point. In preferred embodiments, the weight of the layer increases by less than about 5%, or less than about 1% upon exposure to such environmental factors at about 1-hour, about 4-hour, about 6-hour, about 12-hour, about 24-hour, about 30-hour, about 36-hour, about 48-hour, or between 48 hours and one week time point. In further preferred embodiments, the weight of the layer increases by less than about 0.5% upon exposure to such environmental factors at about 1-hour, about 4-hour, about 6-hour, about 12-hour, about 24-hour, about 30-hour, about 36-hour, about 48-hour, or between 48 hours and one week time point.

In certain embodiments, the linear siloxane polymers are dual functional low- to moderate- to high- molecular weight linear siloxane polymers containing one hydride functionality terminus and one unsaturated functionality terminus. The hydride functionality and the unsaturated functionality are each attached to a different silicon atom on opposite ends of the linear siloxane polymer. These materials are liquids having viscosities in the range of about 5 to about 100,000 cSt and low polydispersities. These materials are liquids having viscosities in the range of about 3 to about 200,000 cSt and low polydispersities. These materials are liquids having viscosities in the range of about 2 to about 400,000 cSt and low polydispersities. These materials are liquids having viscosities in the range of about 1 to about 800,000 cSt and low polydispersities. These materials are liquids having viscosities in the range of about 1 to about 1600,000 cSt and low polydispersities. These materials are liquids having viscosities in the range of about 1 to about 3200,000 cSt and low polydispersities.

In certain embodiments, the term “low polydispersity” may be understood to refer to a polydispersity less than about 1.6, more preferably less than about 1.4, most preferably less than about 1.2. The degree of polymerization of the siloxanes is preferably greater than 6, more preferably 6 to about 1000, most preferably about 10 to about 200. It is essential that the dual functional materials have a ratio of substantially 1:1 of unsaturated group termini to hydride group termini within each polymer molecule. In certain embodiments, the term “substantially 1:1” means that the ratio is within about 5% of 1:1, more preferably within about 3% of 1:1. The ratio of unsaturated group termini to hydride group termini may be estimated based on a combination of 1H NMR, GPC, or step-growth polymerization data. The most effective method for achieving this 1:1 ratio is a method of “living” anionic ring-opening polymerization (“living” AROP), described below.

In certain embodiments, dual functional low- to moderate- to high-molecular weight linear siloxane polymers have the molecular weights from about 3200 daltons to about 160000 daltons. In certain embodiments, dual functional low- to moderate- to high-molecular weight linear siloxane polymers have the molecular weights from about 4700 daltons to about 235000 daltons. In certain embodiments, dual functional low- to moderate- to high-molecular weight linear siloxane polymers have the molecular weights from about 6180 daltons to about 309000 daltons. In certain embodiments, dual functional low- to moderate- to high-molecular weight linear siloxane polymers have the molecular weights from about 3200 daltons to about 32000 daltons. In certain embodiments, dual functional low- to moderate- to high-molecular weight linear siloxane polymers have the molecular weights from about 4700 daltons to about 47000 daltons. In certain embodiments, dual functional low- to moderate- to high-molecular weight linear siloxane polymers have the molecular weights from about 6180 daltons to about 61800 daltons.

The unsaturated functionality is not particularly limited, and may be, for example, a vinyl, styryl, allyl, methallyl, hexenyl, octenyl or alkynyl group. The preferred unsaturated functionality is a vinyl group. The siloxane backbone may be, for example, a dialkylsiloxane derived from a cyclotrisiloxane, such as dimethylsiloxane, ethylmethylsiloxane, diethylsiloxane, dimethylsilylethylsiloxane, trifluoropropylmethylsiloxane, or aromatic substituted siloxanes such as diphenylsiloxane or phenylmethylsiloxane. Other possibilities resulting in siloxane-hydrocarbon copolymers consistent with “living” AROP include 2,2,5,5-tetramethyl-2,5-disila-1-oxacyclopentane and related ring-strained systems.

In certain embodiments, the bifunctional organopolysiloxane polymer has a dimethylsiloxane backbone, an unsaturated group and a hydride group, as shown in formula (IV):

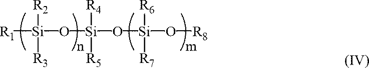

wherein one of R₁, R₄, R₅ and R₈ is an unsaturated group;
one of R₁, R₄, R₅ and R₈ is a hydride group;
the rest of R₁, R₄, R₅ and R₈ are C₁₋₂₀ alkyl groups;
R₂, R₃, R₆ and R₇ are C₁₋₂₀ alkyl groups; and
the sum of n and m is between 2 to 50.

In certain embodiments, the bifunctional organopolysiloxane polymer has a dimethylsiloxane backbone and a vinyl group as the unsaturated functionality, as shown in formula (V):

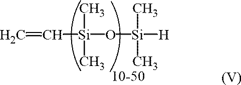

In certain embodiments, the bifunctional organopolysiloxane polymer includes monounsaturated-monohydride terminated siloxanes. In certain embodiments, the monounsaturated-monohydride terminated siloxanes are stable, non-flammable liquids of moderate viscosity, which can be transferred by pouring, pumping, or by means of a syringe. In certain embodiments, the siloxanes may be compounded with a variety of reinforcing agents and thixotropic and non-thixotropic fillers, including fumed silica. In certain embodiments, monovinylphenyl-terminated siloxanes are advantageous due to the ability to control the optical properties (refractive index) and thermal properties.

In certain embodiments, the bifunctional organopolysiloxane polymer can be prepared according to the methods described in the disclosure of U.S. Pat. No. 8,952,118, the disclosure of which is incorporated herein by reference in its entirety. The linear siloxane polymers can be also prepared according to other methods apparent to those of skill in the art.

In one embodiment, the composition provided herein can be stored at about -5, 0, 5, 10, 15, 25, 30, 35 or 40° C. without visible changes. In one embodiment, the composition provided herein can be stored for about 30, 60, 90, 120 or 180 days or for about 1, 2 or 3 years without visible changes. In one embodiment, the composition provided herein can be stored with light. In one embodiment, the composition provided herein is stored without light. In one embodiment, the composition provided herein is stored in a light-proof container. In one embodiment, the composition provided herein is stored in a sound-proof container. In one embodiment, the composition provided herein is stored in a shock-proof container. In one embodiment, the composition provided herein is stored in a thermo-insulated container. In one embodiment, the composition provided herein is stored in an electromagnetically shielded container.

Provided herein are compositions that can be used to form a film over the skin of a subject. In certain embodiments, the resulting film has certain properties that are described herein. In certain embodiments, the film can be used for cosmetic and therapeutic applications.

More specifically, provided herein is a composition that can be used as a single formulation to be applied to, e.g., the skin of a subject where it forms a film over the skin of the subject. In certain embodiments, a formulation provided herein includes at least one transition metal capable of catalyzing the cross-linking reaction between a vinyl functionalized organopolysiloxane and a hydride functionalized polysiloxane. Such a formulation can be configured such that the transition metal is prevented from catalyzing the cross-linking reaction before film-formation is desired (e.g., before application to the skin of a subject) thereby allowing formulation of the catalyst and the monomers in a single composition. In certain embodiments, the formulation can include at least one ligand that prevents the transition metal from catalyzing the cross-linking reaction. Once film formation is desired, the activity of the ligand to prevent the cross-linking reaction can be reduced or eliminated by different means depending on the nature of the ligand as described hereinbelow. In certain embodiments, the formulation can include at least one encapsulating agent that prevents the transition metal from catalyzing the cross-linking reaction or the hydride functionalized polysiloxane from freely interacting with vinyl functionalized organopolysiloxane in the vicinity of the transition metal. Once film formation is desired, the activity of the encapsulating agent to prevent the cross-linking reaction can be reduced or eliminated by different means depending on the nature of the encapsulating agent as described hereinbelow.

More specifically, provided herein is a composition that can be used as a single formulation to be applied to, e.g., the skin of a subject where it forms a film over the skin of the subject. In certain embodiments, a formulation provided herein includes at least one transition metal capable of catalyzing the hydrosilylation step-growth polymerization reaction of the bifunctional organopolysiloxane polymer. Such a formulation can be configured such that the transition metal is prevented from catalyzing the hydrosilylation step-growth polymerization reaction before film-formation is desired (e.g., before application to the skin of a subject) thereby allowing formulation of the catalyst and the bifunctional organopolysiloxane polymer in a single composition. In certain embodiments, the formulation can include at least one ligand that prevents the transition metal from catalyzing the hydrosilylation step-growth polymerization reaction. Once film formation is desired, the activity of the ligand to prevent the hydrosilylation step-growth polymerization reaction can be reduced or eliminated by different means depending on the nature of the ligand as described herein below. In certain embodiments, the formulation can include at least one encapsulating agent that prevents the transition metal from catalyzing the hydrosilylation step-growth polymerization reaction of the bifunctional organopolysiloxane polymer. Once film formation is desired, the activity of the encapsulating agent to prevent the cross-linking reaction can be reduced or eliminated by different means depending on the nature of the encapsulating agent as described herein below.

### 1.5 Environment-Responsive Agent

In one embodiment, the environment-responsive agent is an ambient responsive agent, photo-responsive agent, sound-responsive agent, pressure-responsive agent, heat-responsive agent, water-responsive, bodily fluid-responsive, saliva-responsive, chemical-responsive, or electromagnetic wave-responsive agent. In one embodiment, the environment-responsive responsive agent is a volatile agent. In one embodiment, the ambient responsive agent is a volatile agent. In one embodiment, when the environment-responsive agent is described herein as being “delivered out” or “transported out” or facilitate out-transport of one or more beneficial agents, this refers to a part of the environment-responsive agent thereof, and does not require that all of the environment-responsive agent or one or more beneficial agents thereof be “delivered out” or “transported out.” The environment-responsive agent of interest herein can be in any suitable form including, but not limited to: solids, liquids, gases, semi-solids, gels, encapsulates, wicks, and carrier materials, such as porous materials impregnated with or containing the environment-responsive material, and combinations thereof.

In one embodiment, the environment-responsivevolatile agent is transported out or facilitate out-transport of one or more beneficial agents in a controlled manner. In one embodiment, the environment-responsivevolatile agent is transported out of the composition or facilitate out-transport of one or more beneficial agents from the composition by convection. In one embodiment, the environment-responsivevolatile agent is transported out of the composition or facilitate out-transport of one or more beneficial agents from the composition by diffusion.

In one embodiment, the environment-responsive agent is transferred out of the composition or facilitate out-transport of one or more beneficial agents from the composition provided herein by evaporation. In one embodiment, the environment-responsive agent is transferred out of the composition or facilitate out-transport of one or more beneficial agents from the composition provided herein by applying pressure. In one embodiment, the environment-responsive agent is transferred out of the composition or facilitate out-transport of one or more beneficial agents from the composition provided herein by exposure to a sound, heat or light. In one embodiment, the environment-responsive agent is transferred out of the composition or facilitate out-transport of one or more beneficial agents from the composition provided herein by exposure to a chemical, water, bodily-fluid, or saliva. In one embodiment, the environment-responsive agent is transferred out of the composition or facilitate out-transport of one or more beneficial agents from the composition provided herein by absorbing the environment-responsive agent into another phase. In one embodiment, the environment-responsive agent is transferred out of the composition or facilitate out-transport of one or more beneficial agents from the composition provided herein by absorbing the environment-responsive agent into the skin of a subject. In one embodiment, the environment-responsive agent is transferred out of the composition or facilitate out-transport of one or more beneficial agents from the composition provided herein by absorbing the environment-responsive agent into another ingredient forming a complex. In one embodiment, the environment-responsive agent is transferred out of the composition or facilitate out-transport of one or more beneficial agents from the composition provided herein by heating the composition. In one embodiment, the environment-responsive agent is transferred out of the composition or facilitate out-transport of one or more beneficial agents from the composition provided herein by cooling the composition. In one embodiment, the environment-responsive agent is transferred out of the composition or facilitate out-transport of one or more beneficial agents from the composition provided herein by using heat. In one embodiment, the environment-responsive agent is transferred out of the composition or facilitate out-transport of one or more beneficial agents from the composition provided herein by using heat generated with a blow-dry. In one embodiment, the environment-responsive agent is transferred out of the composition or facilitate out-transport of one or more beneficial agents from the composition provided herein by applying ultrasound to the composition. In one embodiment, the environment-responsive agent is transferred out of the composition or facilitate out-transport of one or more beneficial agents from the composition provided herein by exposure to radiative transfer of electomagetic waves. In one embodiment, the environment-responsive agent is transferred out of the composition or facilitate out-transport of one or more beneficial agents from the composition provided herein by applying electromagnetic waves to the composition. In one embodiment, the environment-responsive agent is transferred out of the composition or facilitate out-transport of one or more beneficial agents from the composition provided herein by applying sun light to the composition. In one embodiment, the environment-responsive agent is transferred out of the composition or facilitate out-transport of one or more beneficial agents from the composition provided herein by applying visible light to the composition. In one embodiment, the environment-responsive agent is transferred out of the composition or facilitate out-transport of one or more beneficial agents from the composition provided herein by applying ultraviolet light to the composition. In one embodiment, the environment-responsive agent is transferred out of the composition or facilitate out-transport of one or more beneficial agents from the composition provided herein by applying infrared radiation to the composition. In one embodiment, the environment-responsive agent is transferred out of the composition or facilitate out-transport of one or more beneficial agents from the composition provided herein by applying water to the composition. In one embodiment, the environment-responsive agent is transferred out of the composition or facilitate out-transport of one or more beneficial agents from the composition provided herein by applying bodily fluid to the composition. In one embodiment, the environment-responsive agent is transferred out of the composition or facilitate out-transport of one or more beneficial agents from the composition provided herein by applying saliva to the composition. In one embodiment, the environment-responsive agent is transferred out of the composition or facilitate out-transport of one or more beneficial agents from the composition provided herein by applying a chemical to the composition.

In one embodiment, the environment-responsive agent is transferred out or facilitate out-transport of one or more beneficial agents from the composition in a gas form, a liquid form, a semi-solid form, a solid form or a mixture thereof. In one embodiment, the environment-responsive agent is transferred out or facilitate out-transport of one or more beneficial agents from the composition in a gas form, e.g., a volatile agent. In one embodiment, the environment-responsive agent is transferred out or facilitate out-transport of one or more beneficial agents from the composition in a liquid form, e.g., a wetting agent. In one embodiment, the environment-responsive agent is transferred out or facilitate out-transport of one or more beneficial agents from the composition in a semi-solid form, e.g., a lubricating agent. In one embodiment, the environment-responsive agent is transferred out or facilitate out-transport of one or more beneficial agents from the composition in a solid form, e.g., airbourne particulates, and sprinkle dusts.

In one embodiment, the environment-responsive agent is selected from a group consisting of cosmetic agents, therapeutic agents, stimuli-responsive agents, sensing agents, drug-delivery agents, optical agents, coloring agents, pigments, scattering agents, sorbing agents, temperature-active agents, heat-active agents, UV-active agents, light-active agents, sound-active agents, pressure-active agents, motion-active agents, radioactive agents, electrical agents, and magnetic agents.

The term “volatile agent” as used herein, refers to a material included of one or more materials that is vaporizable, or includes a material that is vaporizable. The term “volatile agent”, thus, includes (but is not limited to) compositions that are included entirely of a single volatile material. The term “volatile agent,” as used herein, includes, but is not limited to pleasant or savory smells, and, thus, also encompasses materials that function as insecticides, air fresheners, deodorants, aromacology, aromatherapy, insecticides, or any other material that acts to condition, modify, or otherwise charge the atmosphere or to modify the environment. It should be understood that certain volatile agents including, but not limited to perfumes, aromatic materials, and scented materials, will often be included of one or more volatile materials (which may form a unique included of a collection of volatile materials). It should be understood that the term “volatile agent” refers to compositions that have at least one volatile component, and it is not necessary for all of the component materials of the volatile composition to be volatile. The volatile agent described herein may, thus, also have non-volatile components. It should also be understood that when the volatile agent is described herein as being “delivered out” or “transported out,” this refers to the volatilization of the volatile agent thereof, and does not require that the non-volatile components thereof be “delivered out” or “transported out.” The volatile agent of interest herein can be in any suitable form including, but not limited to: solids, semi-solids, liquids, gels, encapsulates, wicks, and carrier materials, such as porous materials impregnated with or containing the volatile material, and combinations thereof.

In one embodiment, the environment-responsive agent is a volatile chemical. In one embodiment, the environment-responsive agent has a vapor pressure of at least about 0.1, at least about 1, at least about 2, at least about 5, at least about 10, at least about 15, at least about 20, at least about 25, at least about 30, at least about 35, at least about 40, or at least about 45 mm Hg at 20° C. In one embodiment, the environment-responsive agent has a vapor pressure of at least about 0.1, at least about 1, at least about 5, at least about 15, at least about 30, or at least about 45 mm Hg at 20° C. In one embodiment, the environment-responsive agent has a vapor pressure of at least about 0.1 mm Hg at 20° C. In one embodiment, the environment-responsive agent has a vapor pressure of at least about 15 mm Hg at 20° C. In one embodiment, the environment-responsive agent has a vapor pressure of at least about 30 mm Hg at 20° C. In one embodiment, the environment-responsive agent has a vapor pressure of at least about 45 mm Hg at 20° C. In one embodiment, the environment-responsive agent has a vapor pressure of at least about 0.1 mm Hg at 20° C. as determined by U.S. EPA Reference Method 24. In one embodiment, the environment-responsive agent has a vapor pressure of at least about 0.1 mm Hg at 20° C. as determined by U.S. EPA Reference Method 24, available at https://www.epa.gov/sites/production/files/2017-08/documents/method_24.pdf (accessed Feb. 17, 2020).

In one embodiment, the environment-responsive agent is in a liquid form, a solid form, a semi-solid form, or a gas form at room temperature. In one embodiment, the environment-responsive agent may evaporate or sublimate from the liquid or solid or semi-solid form and enter the surrounding air at body temperature.

In one embodiment, the environment-responsive agent is transported out in a controlled manner. In one embodiment, about 10% by weight of the environment-responsive agent is transported out of the film in 1 hour. In one embodiment, about 15% by weight of the environment-responsive agent is transported out of the film in 3 hours. In one embodiment, about 20 % by weight of the environment-responsive agent is transported out of the film in 6 hours. In one embodiment, about 40% by weight of the environment-responsive agent is transported out of the film in 12 hours. In one embodiment, about 60% by weight of the environment-responsive agent is transported out of the film in 24 hours. In one embodiment, about 80% by weight of the environment-responsive agent is transported out of the film in 48 hours. In one embodiment, about 80% by weight of the environment-responsive agent is transported out of the film in 72 hours. In one embodiment, about 80% by weight of the environment-responsive agent is transported out of the film in 4 days. In one embodiment, about 80% by weight of the environment-responsive agent is transported out of the film in 5 days. In one embodiment, about 80% by weight of the environment-responsive agent is transported out of the film in 6 days. In one embodiment, about 80% by weight of the environment-responsive agent is transported out of the film in 1 week. In one embodiment, about 80% by weight of the environment-responsive agent is transported out of the film in 8 days. In one embodiment, about 80% by weight of the environment-responsive agent is transported out of the film in 9 days. In one embodiment, about 80% by weight of the environment-responsive agent is transported out of the film in 10 days. In one embodiment, about 80% by weight of the environment-responsive agent is transported out of the film in 11 days. In one embodiment, about 80% by weight of the environment-responsive agent is transported out of the film in 12 days. In one embodiment, about 80% by weight of the environment-responsive agent is transported out of the film in 13 days. In one embodiment, about 80% by weight of the environment-responsive agent is transported out of the film in 2 weeks. In one embodiment, the environment-responsive agent is transported out on average in about 1 part per trillion per minute by weight. In one embodiment, the environment-responsive agent is transported out from the composition into the surrounding atmosphere on average in about 1 part per billion per minute by weight. In one embodiment, the environment-responsive agent is transported out from the composition into the surrounding atmosphere on average in about 1 part per million per minute by weight. In one embodiment, the environment-responsive agent is transported out from the composition into the surrounding atmosphere on average in about 0.01% per minute by weight. In one embodiment, the environment-responsive agent is transported out from the composition into the surrounding atmosphere on average in about 0.1% per minute by weight. In one embodiment, the environment-responsive agent is transported out from the composition into the surrounding atmosphere on average in about 1% per minute by weight. In one embodiment, the environment-responsive agent is transported out from the composition into the surrounding atmosphere on average in about 10% per minute by weight. In one embodiment, the amount of the transferred-out environment-responsive agent is determined by gas chromatography (GC). In one embodiment, the amount of the transferred-out environment-responsive agent is determined by inductively-coupled plasma (ICP).

In one embodiment, the volatile agent is fragrance, a volatile active, a drug, a beneficial agent, an environment responsive agent, an active that attracts animals, an active that repels animals, a pheromone, an insect repellent, a mosquito repellent, a cooling agent, a heating agent, an antihistamine, an odor masking active, a humidity control agent, an inhalant, an antidepressant, or nicotine.

The fragrance includes, but is not limited to aroma compounds, flavors, odorants, and odorizers whose molecular structure containsester, alcohol, linear terpene, cyclic terpene, aromatic, amine, aldehyde, ketone, lactone, thiol, ancombinations thereof (e.g. geranyl acetate, methyl formate, methyl acetate, methyl propionate, methyl butyrate, ethyl acetate, ethyl butyrate, isoamyl acetate, pentyl butyrate, pentyl pentanoate, octyl acetate, benzyl acetate, methyl anthranilate, hexyl acetate, myrcene, geraniol, neol, citral, citronellal, citronellol, linalool, nerolidol, ocimene, limonene, camphor, menthol, carvone, terpineol, alpha-lonone, thujone, eucalyptol, jasmone, eugenol, acetone, ethanol, benzaldehyde, formaldehyde, cinnamaldehyde, ethyl maltol, vanillin, anisole, anethole, estragole, thymol, trimethylamine, putrescine, cadaverine, pyridine, indole, skatole, furaneol, 1-hexanol, cis-3-hexen-1-ol, acetaldehyde, hexanal, cis-3-hexanal, furfural, hexyl cinnamaldehyde, isovaleraldehyde, anisic aldehyde, fructone, ethyl methyphenylglycidate, alpha-methyllbenzyl acetate, cyclopentadecanone, dihydrojasmone, octenone, acetyl pyrroline, acetyl tetrahydropyridine, decalactone, nonalactone, octalactone, jasmine lactone, massoia lactone, wine lactone, sotolon, thioacetone, allyl thiol, methylthio methanethiol, ethanethiol, methyl propanethiol, butane thil, grapefruit mecaptan, methanethiol, furanylmethanethiol, furfuryl mercaptan, benzyl mercaptan, methylphosphine, dimethylphosphine, phosphine, diacetyl, acetoin, nerolin, tetrahydrothiophene, trichloroanisole, substituted pyrazines, methylene chloride, benzyl alcohol and phthalates.

The inhalant includes, but is not limited to: aliphatic hydrocarbons (e.g., petroleum products, gasoline, kerosene, propane and butane), aromatic hydrocarbons (e.g., toluene and xylene), ketones (e.g., acetone), haloalkanes (e.g., hydrofluorocarbons, chlorofluorocarbons, 1,1,1-trichloroethane, trichloroethylene, chloroform), nitrites (e.g., alkyl nitrites and amyl nitrite) and nitrous oxide.

The antidepressant includes, but is not limited to: Citalopram (Celexa, Cipramil); Escitalopram (Lexapro, Cipralex); Fluoxetine (Prozac, Sarafem); Fluvoxamine (Luvox, Faverin); Paroxetine (Paxil, Seroxat); Sertraline (Zoloft, Lustral); Desvenlafaxine (Pristiq); Duloxetine (Cymbalta); Levomilnacipran (Fetzima); Milnacipran (Ixel, Savella); Venlafaxine (Effexor); Vilazodone (Viibryd); Vortioxetine (Trintellix); Nefazodone (Dutonin, Nefadar, Serzone); Trazodone (Desyrel); Atomoxetine (Strattera); Reboxetine (Edronax); Teniloxazine (Lucelan, Metatone); Viloxazine (Vivalan); Bupropion (Wellbutrin); Amitriptyline (Elavil, Endep); Amitriptylinoxide (Amioxid, Ambivalon, Equilibrin); Clomipramine (Anafranil); Desipramine (Norpramin, Pertofrane); Dibenzepin (Noveril, Victoril); Dimetacrine (Istonil); Dosulepin (Prothiaden); Doxepin (Adapin, Sinequan); Imipramine (Tofranil); Lofepramine (Lomont, Gamanil); Melitracen (Dixeran, Melixeran, Trausabun); Nitroxazepine (Sintamil); Nortriptyline (Pamelor, Aventyl); Noxiptiline (Agedal, Elronon, Nogedal); Opipramol (Insidon); Pipofezine (Azafen/Azaphen); Protriptyline (Vivactil); Trimipramine (Surmontil); Amoxapine (Asendin); Maprotiline (Ludiomil); Mianserin (Tolvon); Mirtazapine (Remeron); Setiptiline (Tecipul); Isocarboxazid (Marplan); Phenelzine (Nardil); Tranylcypromine (Pamate); Selegiline (Eldepryl, Zelapar, Emsam); Caroxazone (Surodil, Timostenil); Metralindole (Inkazan); Moclobemide (Aurorix, Manerix); Pirlindole (Pirazidol); Toloxatone (Humoryl); Eprobemide (Befol); minaprine (Brantur, Cantor); Bifemelane (Alnert, Celeport); Agomelatine (Valdoxan); Esketamine (Spravato); Ketamine (Ketalar); Tandospirone (Sediel); Tianeptine (Stablon, Coaxil); α-Methyltryptamine (Indopan); Etryptamine, α-Ethyltryptamine (αET), (Monase); Indeloxazine (Elen, Noin); Medifoxamine (Cledial, Gerdaxyl); Oxaflozane (Conflictan); Pivagabine (Tonerg); Ademetionine, S-Adenosyl-L-methionine (SAMe), (Heptral, Transmetil, Samyl); Hypericum perforatum (Jarsin, Kira, Movina); Oxitriptan, 5-Hydroxytryptophan (5-HTP), (Cincofarm, Levothym, Triptum); Rubidium chloride (Rubinorm); Tryptophan (Tryptan, Optimax, Aminomine); Magnesium; Acetylcarnitine; Amisulpride (Solian); Aripiprazole (Abilify); Brexpiprazole (Rexulti); Lurasidone (Latuda); Olanzapine (Zyprexa); Quetiapine (Seroquel); Risperidone (Risperdal); Buspirone (Buspar); Lithium (Eskalith, Lithobid); Modafinil; Thyroxine; Triiodothyronine; Minocycline; Amitriptyline, chlordiazepoxide; benzodiazepine (Limbitrol); Amitriptyline, perphenazine (Etafron); Flupentixol, melitracen (Deanxit); Olanzapine, fluoxetine (Symbyax); Tranylcypromine and trifluoperazine.

### 1.6 Ligand

Provided herein is a composition for application to skin of a subject, wherein the composition includes (a) an unsaturated organopolymer; (b) a hydride functionalized polysiloxane; (c) an environment-responsive agent that is capable of transporting out of the composition or facilitate out-transport of one or more beneficial agents from the composition after the composition is applied to a subject; (d) a catalyst, wherein the catalyst is capable of cross-linking the unsaturated organopolymer and the hydride functionalized polysiloxane thereby forming a film over the skin of a subject; and (e) at least one ligand at a concentration sufficient to slow down cross-linking reaction between the unsaturated organopolymer and the hydride functionalized polysiloxane, such that these components can be formulated and stored together as a mixture without significant cross-linking. Provided herein is a composition for application to skin of a subject, wherein the composition includes (a) a vinyl functionalized organopolysiloxane; (b) a hydride functionalized polysiloxane; (c) a volatile agent that is capable of transporting out of the composition or facilitate out-transport of one or more beneficial agents from the composition after the composition is applied to a subject; (d) a catalyst, wherein the catalyst is capable of cross-linking the vinyl functionalized organopolysiloxane and the hydride functionalized polysiloxane thereby forming a film over the skin of a subject; and (e) at least one ligand at a concentration sufficient to slow down cross-linking reaction between the vinyl functionalized organopolysiloxane and the hydride functionalized polysiloxane, such that these components can be formulated and stored together as a mixture without significant cross-linking.

Provided herein are compositions for the formation of a film over the skin of a subject, including: a) a bifunctional organopolysiloxane polymer having one unsaturated group and one hydride group; b) an environment-responsive agent that is capable of transporting out of the composition or facilitate out-transport of one or more beneficial agents from the composition after the composition is applied to a subject; (c) a catalyst, wherein the catalyst is capable of catalyzing hydrosilylation step-growth polymerization reaction of the bifunctional organopolysiloxane polymer thereby forming a film over the skin of a subject; and (d) at least one ligand at a concentration sufficient to slow down the hydrosilylation step-growth polymerization reaction of the bifunctional organopolysiloxane polymer, such that these components can be formulated and stored together as a mixture without significant polymerization. Provided herein are compositions for the formation of a film over the skin of a subject, including: a) a bifunctional organopolysiloxane polymer having one unsaturated group and one hydride group; b) a volatile agent that is capable of transporting out of the composition or facilitate out-transport of one or more beneficial agents from the composition after the composition is applied to a subject; (c) a catalyst, wherein the catalyst is capable of catalyzing hydrosilylation step-growth polymerization reaction of the bifunctional organopolysiloxane polymer thereby forming a film over the skin of a subject; and (d) at least one ligand at a concentration sufficient to slow down the hydrosilylation step-growth polymerization reaction of the bifunctional organopolysiloxane polymer, such that these components can be formulated and stored together as a mixture without significant polymerization.

In certain embodiments, the ligand is a chemical or a functional group that binds to a catalyst to form a ligand-catalyst complex.

In one embodiment, the ligand is capable of slowing down the catalytic activity for the crosslinking reaction by which the compositions provided herein form a chemical crosslink network. In one embodiment, the ligand is capable of slowing down the catalytic activity for the hydrosilylation step-growth polymerization reaction. In one embodiment, the ligand slows down the reaction via complexation, or coordination.

The following chemicals may be used as the ligand for use with the compositions and methods provided herein: divinyltetramethyldisilane, linear vinyl siloxanes, cyclic vinyl siloxanes, tris (vinylsiloxy) silanes, tetrakis (vinylsiloxy) silanes and beyond, vinyl ketones and vinyl esters, acetylenic alcohols, sulfides and mercaptans including all their derivatives. Examples of linear vinyl siloxanes include divinyl disiloxane, divinyl trisiloxane, divinyl tetrasiloxane, and beyond (divinyl dimethicone) - including derivatives as examples in divinyl trisiloxane derivatives: 1,5-divinyl-3-phenylpentamethyltrisilxoane; 1,1,5,5-tetramethyl-3,3-diphenyl-1,5-divinyltrisiloxane. Examples of cyclic vinyl siloxanes include trivinyl trimethylcyclotrisiloxane, tetravinyl tetramethylcyclotetrasiloxane, pentavinyl pentamethylcyclopentasiloxane, hexavinyl hexamethylcyclohexasiloxane, and beyond - including derivatives as examples in substitution of methyl to alkyl or alkoxyl such as ethyl or ethoxy. Examples of branched (vinylsiloxy) silanes and their derivatives include tris (vinyldimethylsiloxy) silane, tetrakis (vinyldimethylsiloxy) silane, methacryloxypropyl tris(vinyldimethylsiloxy) silane. Examples of vinyl ketones and vinyl esters and their derivatives include dimethyl fumarate, dimethyl maleate, methyl vinyl ketone, methoxy butanone. Examples of acetylenic alcohols and their derivatives include methyl isobutynol. Examples of sulfides, mercaptans and their derivatives include ethyl mercaptan, diethyl sulfide, hydrogen sulfide, dimethyl disulfide.

In certain embodiments, the ligand is at a concentration sufficient to slow down the cross-linking reaction between the unsaturated organopolymer or vinyl functionalized organopolysiloxane and the hydride functionalized polysiloxane, such that these components can be formulated and stored together as a mixture without significant cross-linking. In certain embodiments, the ligand is at a concentration sufficient to slow down the hydrosilylation step-growth polymerization reaction of the bifunctional organopolysiloxane polymer, such that these components can be formulated and stored together as a mixture without significant hydrosilylation step-growth polymerization. In certain embodiments, the ligand is at a concentration sufficient to slow down the reaction rate of the cross-linking reaction at about 25° C. to 99% of the reaction rate without the ligand. In certain embodiments, the ligand is at a concentration sufficient to slow down the reaction rate of the cross-linking reaction at about 25° C. to 50% of the reaction rate without the ligand. In certain embodiments, the ligand is at a concentration sufficient to slow down the reaction rate of the cross-linking reaction at about 25° C. to 25% of the reaction rate without the ligand. In certain embodiments, the ligand is at a concentration sufficient to slow down the reaction rate of the cross-linking reaction at about 25° C. to 10% of the reaction rate without the ligand. In certain embodiments, the ligand is at a concentration sufficient to slow down the reaction rate of the cross-linking reaction at about 25° C. to about 1% of the reaction rate without the ligand. In certain embodiments, the ligand is at a concentration sufficient to slow down the reaction rate of the cross-linking reaction at about 25° C. to about 0.1% of the reaction rate without the ligand. In certain embodiments, the ligand is at a concentration sufficient to slow down the reaction rate of the cross-linking reaction at about 25° C. to about 0.01% of the reaction rate without the ligand. In certain embodiments, the ligand is at a concentration sufficient to slow down the reaction rate of the cross-linking reaction at about 25° C. to about 0.001% of the reaction rate without the ligand. In certain embodiments, the ligand is at a concentration sufficient to slow down the reaction rate of the cross-linking reaction at about 25° C. to about 0.0001% of the reaction rate without the ligand. In certain embodiments, the ligand is at a concentration sufficient to slow down the reaction rate of the cross-linking reaction at about 25° C. to about 0.00001% of the reaction rate without the ligand. In certain embodiments, the ligand is at a concentration sufficient to slow down the reaction rate of the cross-linking reaction at about 25° C. to about 0.000001% of the reaction rate without the ligand. In certain embodiments, the ligand is at a concentration sufficient to slow down the reaction rate of the cross-linking reaction at about 25° C. to about 0.0000001% of the reaction rate without the ligand.

In certain embodiments, the ligand is at a concentration sufficient to slow down the cross-linking reaction between the unsaturated organopolymer or vinyl functionalized organopolysiloxane and the hydride functionalized polysiloxane, such that these components can be formulated and stored together as a mixture without significant cross-linking. In certain embodiments, the ligand is at a concentration sufficient to slow down the hydrosilylation step-growth polymerization reaction of the bifunctional organopolysiloxane polymer, such that these components can be formulated and stored together as a mixture without significant hydrosilylation step-growth polymerization. In certain embodiments, the ligand is at a concentration sufficient to slow down the reaction rate of the cross-linking reaction at about 5° C. to 99% of the reaction rate without the ligand. In certain embodiments, the ligand is at a concentration sufficient to slow down the reaction rate of the cross-linking reaction at about 5° C. to 50% of the reaction rate without the ligand. In certain embodiments, the ligand is at a concentration sufficient to slow down the reaction rate of the cross-linking reaction at about 5° C. to 25% of the reaction rate without the ligand. In certain embodiments, the ligand is at a concentration sufficient to slow down the reaction rate of the cross-linking reaction at about 5° C. to 10% of the reaction rate without the ligand. In certain embodiments, the ligand is at a concentration sufficient to slow down the reaction rate of the cross-linking reaction at about 5° C. to about 1% of the reaction rate without the ligand. In certain embodiments, the ligand is at a concentration sufficient to slow down the reaction rate of the cross-linking reaction at about 5° C. to about 0.1% of the reaction rate without the ligand. In certain embodiments, the ligand is at a concentration sufficient to slow down the reaction rate of the cross-linking reaction at about 5° C. to about 0.01% of the reaction rate without the ligand. In certain embodiments, the ligand is at a concentration sufficient to slow down the reaction rate of the cross-linking reaction at about 5° C. to about 0.001% of the reaction rate without the ligand. In certain embodiments, the ligand is at a concentration sufficient to slow down the reaction rate of the cross-linking reaction at about 5° C. to about 0.0001% of the reaction rate without the ligand. In certain embodiments, the ligand is at a concentration sufficient to slow down the reaction rate of the cross-linking reaction at about 5° C. to about 0.00001% of the reaction rate without the ligand. In certain embodiments, the ligand is at a concentration sufficient to slow down the reaction rate of the cross-linking reaction at about 5° C. to about 0.000001% of the reaction rate without the ligand. In certain embodiments, the ligand is at a concentration sufficient to slow down the reaction rate of the cross-linking reaction at about 5° C. to about 0.0000001% of the reaction rate without the ligand.

In certain embodiments, the ligand is capable of delaying the hydrosilylation reaction by which the compositions provided herein form a chemical crosslink network. In certain embodiments, the ligand is capable of lowering the reaction rate of the hydrosilylation reaction at about 25° C. to 99% of the reaction rate without the ligand. In certain embodiments, the ligand is capable of lowering the reaction rate of the hydrosilylation reaction at about 25° C. to 50% of the reaction rate without the ligand. In certain embodiments, the ligand is capable of lowering the reaction rate of the hydrosilylation reaction at about 25° C. to 25% of the reaction rate without the ligand. In certain embodiments, the ligand is capable of lowering the reaction rate of the hydrosilylation reaction at about 25° C. to 10% of the reaction rate without the ligand. In certain embodiments, the ligand is capable of lowering the reaction rate of the hydrosilylation reaction at about 25° C. to about 1% of the reaction rate without the ligand. In certain embodiments, the ligand is capable of lowering the reaction rate of the hydrosilylation reaction at about 25° C. to about 0.1% of the reaction rate without the ligand. In certain embodiments, the ligand is capable of lowering the reaction rate of the hydrosilylation reaction at about 25° C. to about 0.01% of the reaction rate without the ligand. In certain embodiments, the ligand is capable of lowering the reaction rate of the hydrosilylation reaction at about 25° C. to about 0.001% of the reaction rate without the ligand. In certain embodiments, the ligand is capable of lowering the reaction rate of the hydrosilylation reaction at about 25° C. to about 0.0001% of the reaction rate without the ligand. In certain embodiments, the ligand is capable of lowering the reaction rate of the hydrosilylation reaction at about 25° C. to about 0.00001% of the reaction rate without the ligand. In certain embodiments, the ligand is capable of lowering the reaction rate of the hydrosilylation reaction at about 25° C. to about 0.000001% of the reaction rate without the ligand. In certain embodiments, the ligand is capable of lowering the reaction rate of the hydrosilylation reaction at about 25° C. to about 0.0000001% of the reaction rate without the ligand.

In certain embodiments, the ligand is capable of delaying the hydrosilylation reaction by which the compositions provided herein form a chemical crosslink network. In certain embodiments, the ligand is capable of lowering the reaction rate of the hydrosilylation reaction at about 5° C. to 99% of the reaction rate without the ligand. In certain embodiments, the ligand is capable of lowering the reaction rate of the hydrosilylation reaction at about 5° C. to 50% of the reaction rate without the ligand. In certain embodiments, the ligand is capable of lowering the reaction rate of the hydrosilylation reaction at about 5° C. to 25% of the reaction rate without the ligand. In certain embodiments, the ligand is capable of lowering the reaction rate of the hydrosilylation reaction at about 5° C. to 10% of the reaction rate without the ligand. In certain embodiments, the ligand is capable of lowering the reaction rate of the hydrosilylation reaction at about 5° C. to about 1% of the reaction rate without the ligand. In certain embodiments, the ligand is capable of lowering the reaction rate of the hydrosilylation reaction at about 5° C. to about 0.1% of the reaction rate without the ligand. In certain embodiments, the ligand is capable of lowering the reaction rate of the hydrosilylation reaction at about 5° C. to about 0.01% of the reaction rate without the ligand. In certain embodiments, the ligand is capable of lowering the reaction rate of the hydrosilylation reaction at about 5° C. to about 0.001% of the reaction rate without the ligand. In certain embodiments, the ligand is capable of lowering the reaction rate of the hydrosilylation reaction at about 5° C. to about 0.0001% of the reaction rate without the ligand. In certain embodiments, the ligand is capable of lowering the reaction rate of the hydrosilylation reaction at about 5° C. to about 0.00001% of the reaction rate without the ligand. In certain embodiments, the ligand is capable of lowering the reaction rate of the hydrosilylation reaction at about 5° C. to about 0.000001% of the reaction rate without the ligand. In certain embodiments, the ligand is capable of lowering the reaction rate of the hydrosilylation reaction at about 5° C. to about 0.0000001% of the reaction rate without the ligand.

In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 30 days. In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 60 days. In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 90 days. In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 120 days. In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 180 days. In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 365 days. In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 730 days. In certain embodiments, the ligand is at a concentration sufficient to slow down the cross-linking reaction or hydrosilylation step-growth polymerization reaction, such that these components can be formulated and stored together as a mixture without significant cross-linking or significant polymerization reaction at about 25° C. for about 3 years.

In certain embodiments, the ligand is at a concentration of about 0.1% by weight of the composition. In certain embodiments, the ligand is at a concentration of about 1% by weight of the composition. In certain embodiments, the ligand is at a concentration of about 10% by weight of the composition. In certain embodiments, the ligand is at a concentration of about 20% by weight of the composition. In certain embodiments, the ligand is at a concentration of about 30% by weight of the composition. In certain embodiments, the ligand is at a concentration of about 40% by weight of the composition. In certain embodiments, the ligand is at a concentration of about 50% by weight of the composition. In certain embodiments, the ligand is at a concentration of about 60% by weight of the composition. In certain embodiments, the ligand is at a concentration of about 70% by weight of the composition. In certain embodiments, the ligand is at a concentration of about 80% by weight of the composition. In certain embodiments, the ligand is at a concentration of about 90% by weight of the composition. In certain embodiments, the ligand is at a concentration of about 95% by weight of the composition. In certain embodiments, the ligand is at a concentration of about 99% by weight of the composition. In certain embodiments, the ligand is at a concentration of about 99.9% by weight of the composition.

In one embodiment, the molar ratio between the ligand and the transition metal is about 10⁷:1. In one embodiment, the molar ratio between the ligand and the transition metal is about 10⁶:1. In one embodiment, the molar ratio between the ligand and transition metal is about 10⁵:1. In one embodiment, the molar ratio between the ligand and the transition metal is about 10⁴:1. In one embodiment, the molar ratio between the ligand and the transition metal is about 10³:1. In one embodiment, the molar ratio between the ligand and the transition metal is about 10²:1. In one embodiment, the molar ratio between the ligand and the transition metal is about 10:1. In one embodiment, the molar ratio between the ligand and the transition metal is about 1:1. In one embodiment, the molar ratio between the ligand and the transition metal is about 1:2. In one embodiment, the molar ratio between the ligand and the transition metal is about 1:5. In one embodiment, the molar ratio between the ligand and the transition metal is about 500:1. In one embodiment, the molar ratio between the ligand and the hydride functionalized polysiloxane is about 10⁷:1. In one embodiment, the molar ratio between the ligand and the hydride functionalized polysiloxane is about 10⁶:1. In one embodiment, the molar ratio between the ligand and hydride functionalized polysiloxane is about 10⁵:1. In one embodiment, the molar ratio between the ligand and the hydride functionalized polysiloxane is about 10⁴:1. In one embodiment, the molar ratio between the ligand and the hydride functionalized polysiloxane is about 10³:1. In one embodiment, the molar ratio between the ligand and the hydride functionalized polysiloxane is about 10²:1. In one embodiment, the molar ratio between the ligand and the hydride functionalized polysiloxane is about 10:1. In one embodiment, the molar ratio between the ligand and the hydride functionalized polysiloxane is about 1:1. In one embodiment, the molar ratio between the ligand and the hydride functionalized polysiloxane is about 1:2. In one embodiment, the molar ratio between the ligand and the hydride functionalized polysiloxane is about 1:5. In one embodiment, the molar ratio between the ligand and the hydride functionalized polysiloxane is about 500:1.

In one embodiment, the ligand is a moderator delaying the hydrosilylation reaction. In one embodiment, the ligand is a moderator delaying the hydrosilylation reaction by complexing with the catalyst. In one embodiment, the ligand is a moderator that complexing with the catalyst reversibly. In one embodiment, the ligand is a moderator that dissociates with the catalyst at higher temperatures, e.g., about 25° C., about 30° C., about 35° C., about 40° C., about 50° C., about 60° C., about 70° C. In one embodiment, the ligand is a moderator that dissociates with the catalyst by evaporation. In one embodiment, the ligand is a moderator that dissociates with the catalyst by solvent extraction. In one embodiment, the ligand is a moderator that dissociates with the catalyst under acoustic wave. In one embodiment, the ligand is a moderator that dissociates with the catalyst under electromagnetic wave. In one embodiment, the ligand is divinyltetramethyldisiloxane, trivinyltetramethyltrisiloxane, trimethylcyclotrisiloxane, tetravinyl tetramethylcyclotetrasiloxane, or dimethyl fumarate. Without being bound by theory, upon dissociation of the ligand from the catalyst, the hydrosilylation reaction is no longer delayed.

In one embodiment, the ligand is a retarder delaying the hydrosilylation reaction. In one embodiment, the ligand is a retarder delaying the hydrosilylation reaction by complexing with the catalyst. In one embodiment, the ligand is a retarder that complexing with the catalyst reversibly. In one embodiment, the ligand is a retarder that dissociates with the catalyst at higher temperatures, e.g., about 25° C., about 30° C., about 35° C., about 40° C., about 50° C., about 60° C., about 70° C. In one embodiment, the ligand is a retarder that dissociates with the catalyst under acoustic wave. In one embodiment, the ligand is a retarder that dissociates with the catalyst under electromagnetic wave. In one embodiment, the ligand is divinyltetramethyldisiloxane, trivinyltetramethyltrisiloxane, trimethylcyclotrisiloxane, tetravinyl tetramethylcyclotetrasiloxanedivinyltetramethyldisiloxane, or dimethyl fumarate. Without being bound by theory, upon dissociation of the ligand from the catalyst, the hydrosilylation reaction is no longer delayed.

In one embodiment, the ligand is an inhibitor preventing the hydrosilylation reaction. In one embodiment, the ligand is an inhibitor preventing the hydrosilylation reaction by complexing with the catalyst. In one embodiment, the ligand is an inhibitor that can be removed to reactivate with the catalyst. In one embodiment, the ligand is an inhibitor that can be removed at higher temperatures, e.g., about 25° C., about 30° C., about 35° C., about 40° C., about 50° C., about 60° C., about 70° C. In one embodiment, the ligand is an inhibitor that can be removed with acoustic wave. In one embodiment, the ligand is an inhibitor that can be removed with electromagnetic wave. In one embodiment, the ligand is a low boiling acetylenic alcohol. In one embodiment, the ligand is methyl-isobutanol.

In certain embodiments, the ligand is capable of slowing down the catalytic activity for hydrosilylation reaction by providing stronger binding interaction to the catalyst, in comparison to other functional moieties, relevant for hydrosilylation.

In certain embodiments, the ligand is capable of slowing down the catalytic activity for hydrosilylation reaction such that at most about 0.1%, 0.5%, 1%, 2%, 5%, 8% or 10% of the functional moieties are reacted over the period of a day, a week, a month, or a year.

In certain embodiments, the ligand is capable of stabilization of the catalyst and spatially separation of the catalyst away from one another. This way, the ligand prevents the catalyst to form larger structure, modifying its catalytic activity.

In certain embodiments, the ligand is capable of stabilization of the catalyst and spatially separation of the catalyst away from hydride functional organopolysiloxanes. This way, the ligand prevents the initiation of intermediate state for hydrosilylation, modifying the catalytic activity of the catalyst.

In certain embodiments, the ligand is capable of stabilization of the catalyst such that at most about 0.01%, 0.05%, 0.1%, 0.5%, 1%, 2%, 5%, 10% or 50% of the catalyst catalyzing the hydrosilylation reaction.

In certain embodiments, the ligand is capable of slowing down the catalytic activity for hydrosilylation reaction by forming a ligand-catalyst complex.

In certain embodiments, the ligand is capable of forming a ligand-catalyst complex such that at least about 99.9%, 99.5%, 99%, 98%, 95%, 92%, 90%, 70%, 50%, 25%, 10% or 5% of the catalyst forms a ligand-catalyst complex.

In certain embodiments, the ligand is capable of forming a ligand-catalyst complex such that at least about 99.9%, 99.5%, 99%, 98%, 95%, 92%, 90%, 70%, 50%, 25%, 10% or 5% of the ligand forms a ligand-catalyst complex.

In certain embodiments, at least about 5% of the ligand forms a ligand-catalyst complex; whereas at least about 99% of the catalyst forms a ligand-catalyst complex.

In one embodiment, the amount of ligand is sufficient to form a ligand-catalyst complex with about 100% of the catalyst. In certain embodiments, the amount of ligand is about 1.1, 1.2, 1.3, 1.4, 1.6, 1.8, 2.0, 2.2, 2.4, 2.6, 2.8, 3.0, 3.4, 3.6, 3.9, 4.0, 4.5, 5, 6, 7, 8, 9, 10, 15, 20, 25, 30, 40, 50, 60, 70, 80, 90 or 100 times by mole of the amount required to form a ligand-catalyst complex with about 100% of the catalyst.

In certain embodiments, the activity of the ligand to slow down the catalytic activity for hydrosilylation reaction can be reduced by decreasing the concentration of the ligand.

In certain embodiments, the activity of the ligand to prevent the slow down the catalytic activity for hydrosilylation reaction can be reduced by decreasing the concentration of the ligand by means of evaporation.

In certain embodiments, the activity of the ligand to slow down the catalytic activity for hydrosilylation reaction can be reduced by decreasing the concentration of the ligand by means of sorption, including physisorption and chemisorption; or adsorption and absorption.

In certain embodiments, the activity of the ligand to slow down the catalytic activity for hydrosilylation reaction can be reduced by decreasing the concentration of the ligand by means of phase separation including solidification, crystallization, precipitation, surface self-segregation, interface self-segregation, phase extraction, phase inversion, or coacervation.

In certain embodiments, the activity of the ligand to slow down the catalytic activity for hydrosilylation reaction can be reduced by decreasing the concentration of the ligand by means of ligand migration such as solvent extraction.

In certain embodiments, the activity of the ligand to slow down the catalytic activity for hydrosilylation reaction can be reduced by decreasing the concentration of the ligand by means of ligand degradation such as chemical oxidation, optical degradation by UV and such.

In certain embodiments, the activity of the ligand to slow down the catalytic activity for hydrosilylation reaction can be reduced by decreasing the concentration of the ligand by means of ligand reconfiguration such as complexation, charge transfer, electron transfer, proton transfer, radical transfer and else.

In certain embodiments, the activity of the ligand to slow down the catalytic activity for hydrosilylation reaction can be reduced by the use of ultrasound to supply vibrational energy to knock the catalyst out of the ligand-catalyst complex.

In certain embodiments, the activity of the ligand to slow down the catalytic activity for hydrosilylation reaction can be reduced by the use of electromagnetic waves that free the catalyst out of the ligand-catalyst complex.

In certain embodiments, the activity of the ligand to slow down the catalytic activity for hydrosilylation reaction can be reduced by the use of temperature as a form of heat or cold that reduces the interactive strength of the ligand-catalyst complex.

In certain embodiments, the activity of the ligand to slow down the catalytic activity for hydrosilylation reaction can be reduced by the use of environments that trigger a phase transition in ligand, impacting the stability of ligand-catalyst complex.

In one embodiment, the ligand is a volatile chemical. In one embodiment, the ligand has a vapor pressure of at least about 0.1, at least about 1, at least about 2, at least about 5, at least about 10, at least about 15, at least about 20, at least about 25, at least about 30, at least about 35, at least about 40, or at least about 45 mm Hg at 20° C. In one embodiment, the ligand has a vapor pressure of at least about 0.1, at least about 1, at least about 5, at least about 15, at least about 30, or at least about 45 mm Hg at 20° C. In one embodiment, the ligand has a vapor pressure of at least about 0.1 mm Hg at 20° C. In one embodiment, the ligand has a vapor pressure of at least about 15 mm Hg at 20° C. In one embodiment, the ligand has a vapor pressure of at least about 30 mm Hg at 20° C. In one embodiment, the ligand has a vapor pressure of at least about 45 mm Hg at 20° C. In one embodiment, the ligand has a vapor pressure of at least about 0.1 mm Hg at 20° C. as determined by U.S. EPA Reference Method 24. In one embodiment, the ligand has a vapor pressure of at least about 0.1 mm Hg at 20° C. as determined by U.S. EPA Reference Method 24, available at https://www.epa.gov/sites/production/files/2017-08/documents/method_24.pdf (accessed Feb. 17, 2020).

In certain embodiments, the volatile ligand can be divinyltetramethyldisilane, divinyldisiloxane, divinyltrisiloxane, trivinyl trimethylcyclotrisiloxane, tetravinyl tetramethylcyclotetrasiloxane, tris (vinyldimethylsiloxy) silane, tetrakis (vinyldimethylsiloxy) silane, dimethyl maleate, methyl vinyl ketone, methyl isobutynol, ethyl mercaptan, diethyl sulfide, hydrogen sulfide, dimethyl disulfide. Without being bound by theory, the activity of the volatile ligand is reduced by exposure to air, wherein the ligand evaporates and the catalyst is set free to catalyze.

In certain embodiments, the ligand is an acoustic-driven ligand. In certain embodiments, the acoustic-driven ligand can be any of the above ligands. Without being bound by theory, the activity of the acoustic-driven ligand is reduced by exposure to ultrasound, wherein the ultrasound supplies vibrational energy to knock the catalyst out of the ligand-catalyst complex. Selection of ultrasound ranges of frequency would regulate the rate of hydrosilylation. In certain embodiment, the catalyst and the ligand may not be necessary for hydrosilylation to proceed, as energy from acoustic cavitation may be sufficient to activate free radicals to initiate the hydrosilylation. In one embodiment, acoustic cavitation activates the hydrogen-terminated silicon surfaces for hydrosilylation.

In certain embodiments, the ligand is an electromagnetic-driven ligand. In certain embodiments, the electromagnetic-driven ligand can be platinum complex of triazine, such as tetrakis (1-phenyl-3-hexyl-triazenido) Pt(IV), Pt(II)-phosphine complex, platinum/oxalate complexs, Pt(II)-bis-(diketonates), dicarbonyl-Pt(IV)R3 complex, sulfoxide-Pt(II) complex. Without being bound by theory, the activity of the electromagnetic-driven ligand is reduced by exposure to electromagnetic wave, wherein the electromagnetic wave such as light, UV, infrared wave, microwave supplies electromagnetic energy to knock the catalyst out of the ligand-catalyst complex.

In certain embodiments, the ligand is a heat-sensitive ligand. In certain embodiments, the heat-sensitive ligand can be platinum complex of triazine such as tetrakis (1-phenyl-3-hexyl-triazenido) Pt(IV), Pt(II)-phosphine complex. Without being bound by theory, the activity of the heat-sensitive ligand is reduced by exposure to direct heat source or heat as a by-product of chemical reaction, microwave, and else; wherein the heat helps release the catalyst out of the ligand-catalyst complex.

In one embodiment, the ligand is 1,3-divinyltetramethyldisiloxane. In one embodiment, the ligand is 1,1,3,3,5,5-hexamethyl-1,5-divinyltrisiloxane. In one embodiment, the ligand is 1,5-divinyl-3-phenylpentamethyltrisiloxane. In one embodiment, the ligand is 1,1,5,5-tetramethyl-3,3-diphenyl-1,5-divinyltrisiloxane. In one embodiment, the ligand is 1,3,5-trivinyl-1,3,5-trimethylcyclotrisiloxane. In one embodiment, the ligand is 2,4,6,8-tetramethyltetravinylcyclotetrasiloxane. In one embodiment, the ligand is 1,3,5,7,9-pentamethyl-1,3,5,7,9-pentavinylcyclopentasiloxane. In one embodiment, the ligand is tris(vinyldimethylsiloxy)methylsilane. In one embodiment, the ligand is tetrakis(vinyldimethylsiloxy)silane. In one embodiment, the ligand is methacryloxypropyltris(vinyldimethylsiloxy)silane. In one embodiment, the ligand is 1,2-divinyltetramethyldisilane. In one embodiment, the ligand is methyl vinyl ketone. In one embodiment, the ligand is dimethyl maleate. In one embodiment, the ligand is dimethyl fumarate. In one embodiment, the ligand is (3E)-4-methoxy-3-buten-2-one. In one embodiment, the ligand is (E)-2-ethylhex-2-enal. In one embodiment, the ligand is pent-1-en-3-one. In one embodiment, in the ligand is maleic acid. In one embodiment, the ligand is alkyl diene, alkyl diyne, alkyl monoyne. In one embodiment, the ligand is butadiene, pentadiene, hexadiene, heptadiene, octadiene. In one embodiment, the ligand is methylbutadiene, methylpentadiene, methylhexadiene, methylheptadience, methyloctadiene. In one embodiment, the ligand is ethylbutadiene, ethylpentadiene, ethylhexadiene, ethylheptadience, ethyloctadiene. In one embodiment, the ligand is dimethylbutadiene, dimethylpentadiene, dimethylhexadiene, dimethylheptadience, dimethyloctadiene, or xylene.

In one embodiment, in the ligand is a polymer having at least one unsaturated group, a function group with one lone-pair electrons or a function group with ability to function as an electron donor. In one embodiment, the ligand is divinyldisiloxane.

In one embodiment, in the ligand is a platinum poison.

In one embodiment, the ligand is a siloxane polymer having at least one unsaturated group. In one embodiment, in the ligand is a vinyl-containing siloxane polymer. In one embodiment, the ligand is a divinyl-containing siloxane polymer. In one embodiment, the ligand is a divinyl-containing disiloxane. In one embodiment, the ligand is divinyl trisiloxane or divinyl tetrasilxoane.

### 1.7 Encapsulating Agent

Provided herein is a composition for application to skin of a subject, wherein the composition includes (a) an unsaturated organopolymer; (b) a hydride functionalized polysiloxane; (c) an environment-responsive agent that is capable of transporting out of the composition or facilitate out-transport of one or more beneficial agents from the composition after the composition is applied to a subject; (d) a catalyst, wherein the catalyst is capable of cross-linking the unsaturated organopolymer and the hydride functionalized polysiloxane thereby forming a film over the skin of a subject; and (e) at least one encapsulating agent, wherein the encapsulating agent slows down or prohibits cross-linking reaction between the unsaturated organopolymer and the hydride functionalized polysiloxane by forming physical or chemical barriers such as microcapsules between the transition metal and hydride functionalized polysiloxane, such that these components can be formulated and stored together as a mixture without significant cross-linking. Provided herein is a composition for application to skin of a subject, wherein the composition includes (a) a vinyl functionalized organopolysiloxane; (b) a hydride functionalized polysiloxane; (c) a volatile agent that is capable of transporting out of the composition or facilitate out-transport of one or more beneficial agents from the composition after the composition is applied to a subject; (d) a catalyst, wherein the catalyst is capable of cross-linking the vinyl functionalized organopolysiloxane and the hydride functionalized polysiloxane thereby forming a film over the skin of a subject; and (e) at least one encapsulating agent, wherein the encapsulating agent slows down or prohibits cross-linking reaction between the vinyl functionalized organopolysiloxane and the hydride functionalized polysiloxane by forming physical or chemical barriers such as microcapsules between the transition metal and hydride functionalized polysiloxane, such that these components can be formulated and stored together as a mixture without significant cross-linking.

Provided herein are compositions for the formation of a film over the skin of a subject, including: a) a bifunctional organopolysiloxane polymer having one unsaturated group and one hydride group; b) an environment-responsive agent that is capable of transporting out of the composition or facilitate out-transport of one or more beneficial agents from the composition after the composition is applied to a subject; (c) a catalyst, wherein the catalyst is capable of catalyzing hydrosilylation step-growth polymerization reaction of the bifunctional organopolysiloxane polymer thereby forming a film over the skin of a subject; and (d) at least one encapsulating agent, wherein the encapsulating agent slows down or prohibits the hydrosilylation step-growth polymerization reaction of the bifunctional organopolysiloxane polymer by forming physical or chemical barriers such as microcapsules between the catalyst and bifunctional organopolysiloxane polymer, such that these components can be formulated and stored together as a mixture without significant polymerization. Provided herein are compositions for the formation of a film over the skin of a subject, including: a) a bifunctional organopolysiloxane polymer having one unsaturated group and one hydride group; b) a volatile agent that is capable of transporting out of the composition or facilitate out-transport of one or more beneficial agents from the composition after the composition is applied to a subject; (c) a catalyst, wherein the catalyst is capable of catalyzing hydrosilylation step-growth polymerization reaction of the bifunctional organopolysiloxane polymer thereby forming a film over the skin of a subject; and (d) at least one encapsulating agent, wherein the encapsulating agent slows down or prohibits the hydrosilylation step-growth polymerization reaction of the bifunctional organopolysiloxane polymer by forming physical or chemical barriers such as microcapsules between the catalyst and bifunctional organopolysiloxane polymer, such that these components can be formulated and stored together as a mixture without significant polymerization.

In certain embodiments, the encapsulating agent is a chemical or a functional group that forms a physical or chemical barrier such as a microcapsule or a self-assembled structure or a network structure with a catalyst or with the hydride functionalized polysiloxane.

In one embodiment, the encapsulating agent is a polysaccharide, protein, lipid or synthetic polymer. In one embodiment, the encapsulating agent is a polysaccharide, wherein the polysaccharide is gum, starch, cellulose, cyclodextrine or chitosan. In one embodiment, the encapsulating agent is a protein, wherein the protein is gelatin, casein or soy protein. In one embodiment, the encapsulating agent is a lipid, wherein the lipid is wax, paraffin or oil. In one embodiment, the encapsulating agent is a synthetic polymer, wherein the synthetic polymer is an acrylic polymer, polyvinyl alcohol or poly(vinylpyrrolidone). In one embodiment, the encapsulating agent is an inorganic material. In one embodiment, the encapsulating agent is an inorganic material, wherein the inorganic material is a silicate, clay or polyphosphate. In one embodiment, the encapsulating agent is a biopolymer or biodegradable polymer. In one embodiment, the encapsulating agent is a biopolymer, wherein the biopolymer is starch. In one embodiment, the encapsulating agent is a biodegradable polymer, wherein the biodegradable polymer is chitosan, hyaluronic acid, a cyclodextrin, alginate, aliphatic polyester or copolymer of lactic and glycolic acids. In one embodiment, the encapsulating agent is an aliphatic polyester, wherein the aliphatic polyester is poly(lactic acid). In one embodiment, the encapsulating agent is a copolymer of lactic and glycolic acids, wherein the copolymer of lactic and glycolic acids is poly(lactic co-glycolic acid). In one embodiment, the encapsulating agent is polyurethane-1, polyurethane-11, polyurethane-14, polyurethane-6, polyurethane-2, polyurethane-18 or their mixtures thereof. In one embodiment, the encapsulating agent is polyurethane-1. In one embodiment, the encapsulating agent is a self-assembled polymer. In one embodiment, the encapsulating agent is a network-forming inorganic dispersion system. In one embodiment, the encapsulating agent is a network-forming inorganic-organic hybrid system.

In certain embodiments, the encapsulating agent is capable of slowing down or prohibiting the catalytic activity for hydrosilylation reaction.

In certain embodiments, the encapsulating agent is at a concentration sufficient to slow down or prohibiting the cross-linking reaction between the unsaturated organopolymer or vinyl functionalized organopolysiloxane and the hydride functionalized polysiloxane, such that these components can be formulated and stored together as a mixture without significant cross-linking. In certain embodiments, the encapsulating agent is at a concentration sufficient to slow down or prohibiting the hydrosilylation step-growth polymerization reaction of the bifunctional organopolysiloxane polymer, such that these components can be formulated and stored together as a mixture without significant hydrosilylation step-growth polymerization. In certain embodiments, the encapsulating agent is at a concentration sufficient to slow down the reaction rate at about 25° C. to 99% of the reaction rate without the encapsulating agent. In certain embodiments, the encapsulating agent is at a concentration sufficient to slow down the reaction rate at about 25° C. to 50% of the reaction rate without the encapsulating agent. In certain embodiments, the encapsulating agent is at a concentration sufficient to slow down the reaction rate at about 25° C. to 25% of the reaction rate without the encapsulating agent. In certain embodiments, the encapsulating agent is at a concentration sufficient to slow down the reaction rate at about 25° C. to 10% of the reaction rate without the encapsulating agent. In certain embodiments, the encapsulating agent is at a concentration sufficient to slow down the reaction rate at about 25° C. to about 1% of the reaction rate without the encapsulating agent. In certain embodiments, the encapsulating agent is at a concentration sufficient to slow down the reaction rate at about 25° C. to about 0.1% of the reaction rate without the encapsulating agent. In certain embodiments, the encapsulating agent is at a concentration sufficient to slow down the reaction rate at about 25° C. to about 0.01% of the reaction rate without the encapsulating agent. In certain embodiments, the encapsulating agent is at a concentration sufficient to slow down the reaction rate at about 25° C. to about 0.001% of the reaction rate without the encapsulating agent. In certain embodiments, the encapsulating agent is at a concentration sufficient to slow down the reaction rate at about 25° C. to about 0.0001% of the reaction rate without the encapsulating agent. In certain embodiments, the encapsulating agent is at a concentration sufficient to slow down the reaction rate at about 25° C. to about 0.00001% of the reaction rate without the encapsulating agent. In certain embodiments, the encapsulating agent is at a concentration sufficient to slow down the reaction rate at about 25° C. to about 0.000001% of the reaction rate without the encapsulating agent. In certain embodiments, the encapsulating agent is at a concentration sufficient to slow down the reaction rate at about 25° C. to about 0.0000001% of the reaction rate without the encapsulating agent. In certain embodiments, the encapsulating agent is at a concentration sufficient to prohibit the reaction rate at about 25° C. to 0% of the reaction rate without the encapsulating agent.

In certain embodiments, the encapsulating agent is at a concentration sufficient to slow down or prohibit the cross-linking reaction between the unsaturated organopolymer or vinyl functionalized organopolysiloxane and the hydride functionalized polysiloxane, such that these components can be formulated and stored together as a mixture without significant cross-linking. In certain embodiments, the encapsulating agent is at a concentration sufficient to slow down or prohibiting the hydrosilylation step-growth polymerization reaction of the bifunctional organopolysiloxane polymer, such that these components can be formulated and stored together as a mixture without significant hydrosilylation step-growth polymerization. In certain embodiments, the encapsulating agent is at a concentration sufficient to slow down the reaction rate of the cross-linking reaction at about 5° C. to 99% of the reaction rate without the encapsulating agent. In certain embodiments, the encapsulating agent is at a concentration sufficient to slow down the reaction rate of the cross-linking reaction at about 5° C. to 50% of the reaction rate without the encapsulating agent. In certain embodiments, the encapsulating agent is at a concentration sufficient to slow down the reaction rate of the cross-linking reaction at about 5° C. to 25% of the reaction rate without the encapsulating agent. In certain embodiments, the encapsulating agent is at a concentration sufficient to slow down the reaction rate of the cross-linking reaction at about 5° C. to 10% of the reaction rate without the encapsulating agent. In certain embodiments, the encapsulating agent is at a concentration sufficient to slow down the reaction rate of the cross-linking reaction at about 5° C. to about 1% of the reaction rate without the encapsulating agent. In certain embodiments, the encapsulating agent is at a concentration sufficient to slow down the reaction rate of the cross-linking reaction at about 5° C. to about 0.1% of the reaction rate without the encapsulating agent. In certain embodiments, the encapsulating agent is at a concentration sufficient to slow down the reaction rate of the cross-linking reaction at about 5° C. to about 0.01% of the reaction rate without the encapsulating agent. In certain embodiments, the encapsulating agent is at a concentration sufficient to slow down the reaction rate of the cross-linking reaction at about 5° C. to about 0.001% of the reaction rate without the encapsulating agent. In certain embodiments, the encapsulating agent is at a concentration sufficient to slow down the reaction rate of the cross-linking reaction at about 5° C. to about 0.0001% of the reaction rate without the encapsulating agent. In certain embodiments, the encapsulating agent is at a concentration sufficient to slow down the reaction rate of the cross-linking reaction at about 5° C. to about 0.00001% of the reaction rate without the encapsulating agent. In certain embodiments, the encapsulating agent is at a concentration sufficient to slow down the reaction rate of the cross-linking reaction at about 5° C. to about 0.000001% of the reaction rate without the encapsulating agent. In certain embodiments, the encapsulating agent is at a concentration sufficient to slow down the reaction rate of the cross-linking reaction at about 5° C. to about 0.0000001% of the reaction rate without the encapsulating agent. In certain embodiments, the encapsulating agent is at a concentration sufficient to prohibit the reaction rate of the cross-linking reaction at about 25° C. to 0% of the reaction rate without the encapsulating agent.

In certain embodiments, the encapsulating agent is capable of delaying or prohibiting the hydrosilylation reaction by which the compositions provided herein. In certain embodiments, the encapsulating agent is capable of lowering the reaction rate of the hydrosilylation reaction at about 25° C. to 99% of the reaction rate without the encapsulating agent. In certain embodiments, the encapsulating agent is capable of lowering the reaction rate of the hydrosilylation reaction at about 25° C. to 50% of the reaction rate without the encapsulating agent. In certain embodiments, the encapsulating agent is capable of lowering the reaction rate of the hydrosilylation reaction at about 25° C. to 25% of the reaction rate without the encapsulating agent. In certain embodiments, the encapsulating agent is capable of lowering the reaction rate of the hydrosilylation reaction at about 25° C. to 10% of the reaction rate without the encapsulating agent. In certain embodiments, the encapsulating agent is capable of lowering the reaction rate of the hydrosilylation reaction at about 25° C. to about 1% of the reaction rate without the encapsulating agent. In certain embodiments, the encapsulating agent is capable of lowering the reaction rate of the hydrosilylation reaction at about 25° C. to about 0.1% of the reaction rate without the encapsulating agent. In certain embodiments, the encapsulating agent is capable of lowering the reaction rate of the hydrosilylation reaction at about 25° C. to about 0.01% of the reaction rate without the encapsulating agent. In certain embodiments, the encapsulating agent is capable of lowering the reaction rate of the hydrosilylation reaction at about 25° C. to about 0.001% of the reaction rate without the encapsulating agent. In certain embodiments, the encapsulating agent is capable of lowering the reaction rate of the hydrosilylation reaction at about 25° C. to about 0.0001% of the reaction rate without the encapsulating agent. In certain embodiments, the encapsulating agent is capable of lowering the reaction rate of the hydrosilylation reaction at about 25° C. to about 0.00001% of the reaction rate without the encapsulating agent. In certain embodiments, the encapsulating agent is capable of lowering the reaction rate of the hydrosilylation reaction at about 25° C. to about 0.000001% of the reaction rate without the encapsulating agent. In certain embodiments, the encapsulating agent is capable of lowering the reaction rate of the hydrosilylation reaction at about 25° C. to about 0.0000001% of the reaction rate without the encapsulating agent. In certain embodiments, the encapsulating agent is capable of prohibiting the reaction rate of the hydrosilylation reaction at about 25° C. to about 0% of the reaction rate without the encapsulating agent.

In certain embodiments, the encapsulating agent is capable of delaying or prohibiting the hydrosilylation reaction by which the compositions provided herein. In certain embodiments, the encapsulating agent is capable of lowering the reaction rate of the hydrosilylation reaction at about 5° C. to 99% of the reaction rate without the encapsulating agent. In certain embodiments, the encapsulating agent is capable of lowering the reaction rate of the hydrosilylation reaction at about 5° C. to 50% of the reaction rate without the encapsulating agent. In certain embodiments, the encapsulating agent is capable of lowering the reaction rate of the hydrosilylation reaction at about 5° C. to 25% of the reaction rate without the encapsulating agent. In certain embodiments, the encapsulating agent is capable of lowering the reaction rate of the hydrosilylation reaction at about 5° C. to 10% of the reaction rate without the encapsulating agent. In certain embodiments, the encapsulating agent is capable of lowering the reaction rate of the hydrosilylation reaction at about 5° C. to about 1% of the reaction rate without the encapsulating agent. In certain embodiments, the encapsulating agent is capable of lowering the reaction rate of the hydrosilylation reaction at about 5° C. to about 0.1% of the reaction rate without the encapsulating agent. In certain embodiments, the encapsulating agent is capable of lowering the reaction rate of the hydrosilylation reaction at about 5° C. to about 0.01% of the reaction rate without the encapsulating agent. In certain embodiments, the encapsulating agent is capable of lowering the reaction rate of the hydrosilylation reaction at about 5° C. to about 0.001% of the reaction rate without the encapsulating agent. In certain embodiments, the encapsulating agent is capable of lowering the reaction rate of the hydrosilylation reaction at about 5° C. to about 0.0001% of the reaction rate without the encapsulating agent. In certain embodiments, the encapsulating agent is capable of lowering the reaction rate of the hydrosilylation reaction at about 5° C. to about 0.00001% of the reaction rate without the encapsulating agent. In certain embodiments, the encapsulating agent is capable of lowering the reaction rate of the hydrosilylation reaction at about 5° C. to about 0.000001% of the reaction rate without the encapsulating agent. In certain embodiments, the encapsulating agent is capable of lowering the reaction rate of the hydrosilylation reaction at about 5° C. to about 0.0000001% of the reaction rate without the encapsulating agent. In certain embodiments, the encapsulating agent is capable of prohibiting the reaction rate of the hydrosilylation reaction at about 25° C. to about 0% of the reaction rate without the encapsulating agent.

In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 30 days. In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 60 days. In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 90 days. In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 120 days. In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 180 days. In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 365 days. In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 730 days. In certain embodiments, the encapsulating agent is at a concentration sufficient to slow down or prohibit the cross-linking reaction or hydrosilylation step-growth polymerization reaction, such that these components can be formulated and stored together as a mixture without significant cross-linking or significant polymerization reaction at about 25° C. for about 3 years.

In certain embodiments, the encapsulating agent is at a concentration of about 1% by weight of the composition. In certain embodiments, the encapsulating agent is at a concentration of about 10% by weight of the composition. In certain embodiments, the encapsulating agent is at a concentration of about 20% by weight of the composition. In certain embodiments, the encapsulating agent is at a concentration of about 30% by weight of the composition. In certain embodiments, the encapsulating agent is at a concentration of about 40% by weight of the composition. In certain embodiments, the encapsulating agent is at a concentration of about 50% by weight of the composition. In certain embodiments, the encapsulating agent is at a concentration of about 60% by weight of the composition. In certain embodiments, the encapsulating agent is at a concentration of about 70% by weight of the composition. In certain embodiments, the encapsulating agent is at a concentration of about 80% by weight of the composition. In certain embodiments, the encapsulating agent is at a concentration of about 90% by weight of the composition. In certain embodiments, the encapsulating agent is at a concentration of about 95% by weight of the composition. In certain embodiments, the encapsulating agent is at a concentration of about 99% by weight of the composition. In certain embodiments, the encapsulating agent is at a concentration of about 99.9% by weight of the composition.

In one embodiment, the molar ratio between the encapsulating agent and the transition metal is about 10⁷:1. In one embodiment, the molar ratio between the encapsulating agent and the transition metal is about 10⁶:1. In one embodiment, the molar ratio between the encapsulating agent and transition metal or hydride functionalized polysiloxane is about 10⁵:1. In one embodiment, the molar ratio between the encapsulating agent and the transition metal is about 10⁴:1. In one embodiment, the molar ratio between the encapsulating agent and the transition metal is about 10³:1. In one embodiment, the molar ratio between the encapsulating agent and the transition metal is about 10²:1. In one embodiment, the molar ratio between the encapsulating agent and the transition metal is about 10:1. In one embodiment, the molar ratio between the encapsulating agent and the transition metal is about 1:1. In one embodiment, the molar ratio between the encapsulating agent and the transition metal is about 1:2. In one embodiment, the molar ratio between the encapsulating agent and the transition metal is about 1:5. In one embodiment, the molar ratio between the encapsulating agent and the transition metal is about 500:1.

In one embodiment, the molar ratio between the encapsulating agent and the hydride functionalized polysiloxane is about 10⁷:1. In one embodiment, the molar ratio between the encapsulating agent and the hydride functionalized polysiloxane is about 10⁶:1. In one embodiment, the molar ratio between the encapsulating agent and transition metal or hydride functionalized polysiloxane is about 10⁵:1. In one embodiment, the molar ratio between the encapsulating agent and the hydride functionalized polysiloxane is about 10⁴:1. In one embodiment, the molar ratio between the encapsulating agent and the hydride functionalized polysiloxane is about 10³:1. In one embodiment, the molar ratio between the encapsulating agent and the hydride functionalized polysiloxane is about 10²:1. In one embodiment, the molar ratio between the encapsulating agent and the hydride functionalized polysiloxane is about 10:1. In one embodiment, the molar ratio between the encapsulating agent and the hydride functionalized polysiloxane is about 1:1. In one embodiment, the molar ratio between the encapsulating agent and the hydride functionalized polysiloxane is about 1:2. In one embodiment, the molar ratio between the encapsulating agent and the hydride functionalized polysiloxane is about 1:5. In one embodiment, the molar ratio between the encapsulating agent and the hydride functionalized polysiloxane is about 500:1.

In one embodiment, the encapsulating agent is a moderator delaying or prohibiting the hydrosilylation reaction. In one embodiment, the encapsulating agent is a moderator delaying or prohibiting the hydrosilylation reaction by forming microcapsules with the catalyst or hydride functionalized polysiloxane. In one embodiment, the encapsulating agent is a moderator delaying or prohibiting the hydrosilylation reaction by forming microcapsules with the catalyst. In one embodiment, the encapsulating agent is a moderator that forms microcapsules with the catalyst or hydride functionalized polysiloxane reversibly. In one embodiment, the encapsulating agent is a moderator that forms microcapsules with the catalyst reversibly. In one embodiment, the encapsulating agent is a moderator that dissociates with the catalyst or hydride functionalized polysiloxane at higher temperatures, e.g., about 25° C., about 30° C., about 35° C., about 40° C., about 50° C., about 60° C., about 70° C. In one embodiment, the encapsulating agent is a moderator that dissociates with the catalyst or hydride functionalized polysiloxane by evaporation. In one embodiment, the encapsulating agent is a moderator that dissociates with the catalyst or hydride functionalized polysiloxane by solvent extraction. In one embodiment, the encapsulating agent is a moderator that dissociates with the catalyst or hydride functionalized polysiloxane under acoustic wave. In one embodiment, the encapsulating agent is a moderator that dissociates with the catalyst or hydride functionalized polysiloxane under electromagnetic wave. Without being bound by theory, upon dissociation of the encapsulating agent from the catalyst or hydride functionalized polysiloxane, the hydrosilylation reaction is no longer delayed.

In one embodiment, the encapsulating agent is a retarder delaying the hydrosilylation reaction. In one embodiment, the encapsulating agent is a retarder delaying the hydrosilylation reaction by complexing with the catalyst or hydride functionalized polysiloxane. In one embodiment, the encapsulating agent is a retarder delaying the hydrosilylation reaction by complexing with the catalyst. In one embodiment, the encapsulating agent is a retarder that forms microcapsules with the catalyst or hydride functionalized polysiloxane reversibly. In one embodiment, the encapsulating agent is a retarder that forms microcapsules with the catalyst reversibly. In one embodiment, the encapsulating agent is a retarder that dissociates with the catalyst or hydride functionalized polysiloxane at higher temperatures, e.g., about 25° C., about 30° C., about 35° C., about 40° C., about 50° C., about 60° C., about 70° C. In one embodiment, the encapsulating agent is a retarder that dissociates with the catalyst or hydride functionalized polysiloxane under acoustic wave. In one embodiment, the encapsulating agent is a retarder that dissociates with the catalyst or hydride functionalized polysiloxane under electromagnetic wave. Without being bound by theory, upon dissociation of the encapsulating agent from the catalyst or hydride functionalized polysiloxane, the hydrosilylation reaction is no longer delayed.

In one embodiment, the encapsulating agent is an inhibitor preventing the hydrosilylation reaction. In one embodiment, the encapsulating agent is an inhibitor preventing the hydrosilylation reaction by forming physical or chemical barriers such as microcapsules with the catalyst or hydride functionalized polysiloxane. In one embodiment, the encapsulating agent is an inhibitor that can be removed to reactivate with the catalyst or hydride functionalized polysiloxane. In one embodiment, the encapsulating agent is an inhibitor that can be removed at higher temperatures, e.g., about 25° C., about 30° C., about 35° C., about 40° C., about 50° C., about 60° C., about 70° C. In one embodiment, the encapsulating agent is an inhibitor that can be removed with acoustic wave. In one embodiment, the encapsulating agent is an inhibitor that can be removed with electromagnetic wave.

In certain embodiments, the encapsulating agent is capable of slowing down or prohibiting the catalytic activity for hydrosilylation reaction such that at most about 0.1%, 0.5%, 1%, 2%, 5%, 8% or 10% of the functional moieties are reacted over the period of a day, a week, a month, or a year.

In certain embodiments, the encapsulating agent is capable of stabilization of the catalyst and spatially separation of the catalyst. In certain embodiments, the encapsulating agent is capable of stabilization of the hydride functionalized polysiloxane and spatially separation of the hydride functionalized polysiloxane. This way, the encapsulating agent prevents the catalyst to form larger structure, modifying its catalytic activity.

In certain embodiments, the encapsulating agent is capable of stabilization of the catalyst or hydride functionalized polysiloxane and spatially separation of the catalyst away from hydride functional organopolysiloxanes and vice versa. This way, the encapsulating agent prevents the initiation of intermediate state for hydrosilylation, modifying the catalytic activity of the catalyst.

In certain embodiments, the encapsulating agent is capable of stabilization of the catalyst such that at most about 0.01%, 0.05%, 0.1%, 0.5%, 1%, 2%, 5%, 10% or 50% of the catalyst catalyzing the hydrosilylation reaction.

In certain embodiments, the encapsulating agent is capable of stabilization of the hydride functionalized polysiloxane such that at most about 0.01%, 0.05%, 0.1%, 0.5%, 1%, 2%, 5%, 10% or 50% of the hydride functionalized polysiloxane remains accessible for the hydrosilylation reaction.

In certain embodiments, the encapsulating agent is capable of slowing down the catalytic activity for hydrosilylation reaction by forming physical or chemical barriers such as microcapsules with the catalyst. In certain embodiments, the encapsulating agent is capable of slowing down the catalytic activity for hydrosilylation reaction by forming physical or chemical barriers such as microcapsules with the hydride functionalized polysiloxane.

In certain embodiments, the encapsulating agent is capable of forming physical or chemical barriers such as microcapsules with the catalyst such that at least about 99.9%, 99.5%, 99%, 98%, 95%, 92%, 90%, 70%, 50%, 25%, 10% or 5% of the catalyst forms microcapsules with the encapsulating agent. In certain embodiments, the encapsulating agent is capable of forming physical or chemical barriers such as microcapsules with the hydride functionalized polysiloxane such that at least about 99.9%, 99.5%, 99%, 98%, 95%, 92%, 90%, 70%, 50%, 25%, 10% or 5% of the encapsulating agent forms microcapsules with the hydride functionalized polysiloxane.

In certain embodiments, at least about 5% of the encapsulating agent forms encapsulating agent-catalyst microcapsules; whereas at least about 99% of the catalyst forms encapsulating agent-catalyst microcapsules.

In one embodiment, the amount of encapsulating agent is sufficient to form encapsulating agent-catalyst microcapsules with about 100% of the catalyst. In certain embodiments, the amount of encapsulating agent is about 1.1, 1.2, 1.3, 1.4, 1.6, 1.8, 2.0, 2.2, 2.4, 2.6, 2.8, 3.0, 3.4, 3.6, 3.9, 4.0, 4.5, 5, 6, 7, 8, 9, 10, 15, 20, 25, 30, 40, 50, 60, 70, 80, 90 or 100 times by mole of the amount required to form encapsulating agent-catalyst microcapsules with about 100% of the catalyst.

In certain embodiments, at least about 5% of the encapsulating agent forms encapsulating agent-hydride functionalized polysiloxane microcapsules; whereas at least about 99% of the catalyst forms encapsulating agent-hydride functionalized polysiloxane microcapsules.

In one embodiment, the amount of encapsulating agent is sufficient to form encapsulating agent-hydride functionalized polysiloxane microcapsules with about 100% of the hydride functionalized polysiloxane. In certain embodiments, the amount of encapsulating agent is about 1.1, 1.2, 1.3, 1.4, 1.6, 1.8, 2.0, 2.2, 2.4, 2.6, 2.8, 3.0, 3.4, 3.6, 3.9, 4.0, 4.5, 5, 6, 7, 8, 9, 10, 15, 20, 25, 30, 40, 50, 60, 70, 80, 90 or 100 times by mole of the amount required to form encapsulating agent-hydride functionalized polysiloxane microcapsules with about 100% of the hydride functionalized polysiloxane.

In certain embodiments, the activity of the encapsulating agent to slow down or prohibit the activity for hydrosilylation reaction can be reduced by decreasing the concentration of the encapsulating agent.

In certain embodiments, the activity of the encapsulating agent to slow down or prohibit the activity for hydrosilylation reaction can be reduced by decreasing the concentration of the encapsulating agent by means of evaporation. In certain embodiments, the activity of the encapsulating agent to slow down or prohibit the activity for hydrosilylation reaction can be reduced by decreasing the concentration of the encapsulating agent by means of sorption, including physisorption and chemisorption; or adsorption and absorption.

In certain embodiments, the activity of the encapsulating agent to slow down or prohibit the activity for hydrosilylation reaction can be reduced by decreasing the concentration of the encapsulating agent by means of phase separation including solidification, crystallization, precipitation, surface self-segregation, interface self-segregation, phase extraction, phase inversion, or coacervation.

In certain embodiments, the activity of the encapsulating agent to slow down or prohibit the activity for hydrosilylation reaction can be reduced by decreasing the concentration of the encapsulating agent by means of encapsulating agent migration such as solvent extraction.

In certain embodiments, the activity of the encapsulating agent to slow down or prohibit the activity for hydrosilylation reaction can be reduced by decreasing the concentration of the encapsulating agent by means of encapsulating agent degradation such as chemical oxidation, optical degradation by UV and such.

In certain embodiments, the activity of the encapsulating agent to slow down or prohibit the activity for hydrosilylation reaction can be reduced by decreasing the concentration of the encapsulating agent by means of encapsulating agent reconfiguration, such as charge transfer, electron transfer, proton transfer, radical transfer and else.

In certain embodiments, the activity of the encapsulating agent to slow down or prohibit the activity for hydrosilylation reaction can be reduced by the use of ultrasound to supply vibrational energy to knock the catalyst or hydride functionalized polysiloxane out of the microcapsules containing encapsulating agent-catalyst or encapsulating agent-hydride functionalized polysiloxane.

In certain embodiments, the activity of the encapsulating agent to slow down or prohibit the activity for hydrosilylation reaction can be reduced by the use of electromagnetic waves that free the catalyst or hydride functionalized polysiloxane out of the microcapsules containing encapsulating agent-catalyst or encapsulating agent-hydride functionalized polysiloxane.

In certain embodiments, the activity of the encapsulating agent to slow down or prohibit the activity for hydrosilylation reaction can be reduced by the use of temperature as a form of heat or cold that reduces the interactive strength of the encapsulating agent-catalyst or encapsulating agent-hydride functionalized polysiloxane microcapsules.

In certain embodiments, the activity of the encapsulating agent to slow down or prohibit the activity for hydrosilylation reaction can be reduced by the use of environments that trigger a phase transition in encapsulating agent, impacting the stability of encapsulating agent-catalyst or encapsulating agent-hydride functionalized polysiloxane microcapsules.

In one embodiment, the encapsulating agent is a volatile chemical. In one embodiment, the encapsulating agent has a vapor pressure of at least about 0.1, at least about 1, at least about 2, at least about 5, at least about 10, at least about 15, at least about 20, at least about 25, at least about 30, at least about 35, at least about 40, or at least about 45 mm Hg at 20° C. In one embodiment, the encapsulating agent has a vapor pressure of at least about 0.1, at least about 1, at least about 5, at least about 15, at least about 30, or at least about 45 mm Hg at 20° C. In one embodiment, the encapsulating agent has a vapor pressure of at least about 0.1 mm Hg at 20° C. In one embodiment, the encapsulating agent has a vapor pressure of at least about 15 mm Hg at 20° C. In one embodiment, the encapsulating agent has a vapor pressure of at least about 30 mm Hg at 20° C. In one embodiment, the encapsulating agent has a vapor pressure of at least about 45 mm Hg at 20° C. In one embodiment, the encapsulating agent has a vapor pressure of at least about 0.1 mm Hg at 20° C. as determined by U.S. EPA Reference Method 24. In one embodiment, the encapsulating agent has a vapor pressure of at least about 0.1 mm Hg at 20° C. as determined by U.S. EPA Reference Method 24, available at https://www.epa.gov/sites/production/files/2017-08/documents/method_24.pdf (accessed Feb. 17, 2020).

Without being bound by theory, the activity of the volatile encapsulating agent is reduced by exposure to air, wherein the encapsulating agent evaporates and the catalyst is set free to catalyze.

In certain embodiments, the encapsulating agent is an acoustic-driven encapsulating agent. In certain embodiments, the acoustic-driven encapsulating agent can be any of the above encapsulating agents. Without being bound by theory, the activity of the acoustic-driven encapsulating agent is reduced by exposure to ultrasound, wherein the ultrasound supplies vibrational energy to knock the catalyst or hydride functionalized polysiloxane out of the encapsulating agent-catalyst or encapsulating agent-hydride functionalized polysiloxane microcapsules. Selection of ultrasound ranges of frequency would regulate the rate of hydrosilylation. In certain embodiment, the catalyst and the encapsulating agent may not be necessary for hydrosilylation to proceed, as energy from acoustic cavitation may be sufficient to activate free radicals to initiate the hydrosilylation. In one embodiment, acoustic cavitation activates the hydrogen-terminated silicon surfaces for hydrosilylation.

In certain embodiments, the encapsulating agent is an electromagnetic-driven encapsulating agent. Without being bound by theory, the activity of the electromagnetic-driven encapsulating agent is reduced by exposure to electromagnetic wave, wherein the electromagnetic wave such as light, UV, infrared wave, microwave supplies electromagnetic energy to knock the catalyst or hydride functionalized polysiloxane out of the microcapsules containing encapsulating agent-catalyst or encapsulating agent-hydride functionalized polysiloxane.

In certain embodiments, the encapsulating agent is a heat-sensitive encapsulating agent. Without being bound by theory, the activity of the heat-sensitive encapsulating agent is reduced by exposure to direct heat source or heat as a by-product of chemical reaction, microwave, and else; wherein the heat helps release the catalyst or hydride functionalized polysiloxane out of the microcapsules containing encapsulating agent-catalyst or encapsulating agent-hydride functionalized polysiloxane.

### 1.8 Beneficial Agent

In one embodiment, the beneficial agent is selected from a group consisting of volatile agents, cosmetic agents, therapeutic agents, stimuli-responsive agents, sensing agents, drug-delivery agents, optical agents, coloring agents, pigments, scattering agents, sorbing agents, temperature-active agents, heat-active agents, UV-active agents, light-active agents, sound-active agents, pressure-active agents, motion-active agents, radioactive agents, electrical agents, and magnetic agents.

In one embodiment, the volatile agent is fragrance, a volatile active, a drug, a beneficial agent, an environment responsive agent, an active that attracts animals, an active that repels animals, a pheromone, an insect repellent, a mosquito repellent, a cooling agent, a heating agent, an antihistamine, an odor masking active, a humidity control agent, an inhalant, an antidepressant, or nicotine.

The fragrance includes, but is not limited to aroma compounds, flavors, odorants, and odorizers whose molecular structure containsester, alcohol, linear terpene, cyclic terpene, aromatic, amine, aldehyde, ketone, lactone, thiol, ancombinations thereof (e.g. geranyl acetate, methyl formate, methyl acetate, methyl propionate, methyl butyrate, ethyl acetate, ethyl butyrate, isoamyl acetate, pentyl butyrate, pentyl pentanoate, octyl acetate, benzyl acetate, methyl anthranilate, hexyl acetate, myrcene, geraniol, neol, citral, citronellal, citronellol, linalool, nerolidol, ocimene, limonene, camphor, menthol, carvone, terpineol, alpha-lonone, thujone, eucalyptol, jasmone, eugenol, acetone, ethanol, benzaldehyde, formaldehyde, cinnamaldehyde, ethyl maltol, vanillin, anisole, anethole, estragole, thymol, trimethylamine, putrescine, cadaverine, pyridine, indole, skatole, furaneol, 1-hexanol, cis-3-hexen-1-ol, acetaldehyde, hexanal, cis-3-hexanal, furfural, hexyl cinnamaldehyde, isovaleraldehyde, anisic aldehyde, fructone, ethyl methyphenylglycidate, alpha-methyllbenzyl acetate, cyclopentadecanone, dihydrojasmone, octenone, acetyl pyrroline, acetyl tetrahydropyridine, decalactone, nonalactone, octalactone, jasmine lactone, massoia lactone, wine lactone, sotolon, thioacetone, allyl thiol, methylthio methanethiol, ethanethiol, methyl propanethiol, butane thil, grapefruit mecaptan, methanethiol, furanylmethanethiol, furfuryl mercaptan, benzyl mercaptan, methylphosphine, dimethylphosphine, phosphine, diacetyl, acetoin, nerolin, tetrahydrothiophene, trichloroanisole, substituted pyrazines, methylene chloride, benzyl alcohol and phthalates.

The inhalant includes, but is not limited to: aliphatic hydrocarbons (e.g., petroleum products, gasoline, kerosene, propane and butane), aromatic hydrocarbons (e.g., toluene and xylene), ketones (e.g., acetone), haloalkanes (e.g., hydrofluorocarbons, chlorofluorocarbons, 1,1,1-trichloroethane, trichloroethylene, chloroform), nitrites (e.g., alkyl nitrites and amyl nitrite) and nitrous oxide.

The antidepressant includes, but is not limited to: Citalopram (Celexa, Cipramil); Escitalopram (Lexapro, Cipralex); Fluoxetine (Prozac, Sarafem); Fluvoxamine (Luvox, Faverin); Paroxetine (Paxil, Seroxat); Sertraline (Zoloft, Lustral); Desvenlafaxine (Pristiq); Duloxetine (Cymbalta); Levomilnacipran (Fetzima); Milnacipran (Ixel, Savella); Venlafaxine (Effexor); Vilazodone (Viibryd); Vortioxetine (Trintellix); Nefazodone (Dutonin, Nefadar, Serzone); Trazodone (Desyrel); Atomoxetine (Strattera); Reboxetine (Edronax); Teniloxazine (Lucelan, Metatone); Viloxazine (Vivalan); Bupropion (Wellbutrin); Amitriptyline (Elavil, Endep); Amitriptylinoxide (Amioxid, Ambivalon, Equilibrin); Clomipramine (Anafranil); Desipramine (Norpramin, Pertofrane); Dibenzepin (Noveril, Victoril); Dimetacrine (Istonil); Dosulepin (Prothiaden); Doxepin (Adapin, Sinequan); Imipramine (Tofranil); Lofepramine (Lomont, Gamanil); Melitracen (Dixeran, Melixeran, Trausabun); Nitroxazepine (Sintamil); Nortriptyline (Pamelor, Aventyl); Noxiptiline (Agedal, Elronon, Nogedal); Opipramol (Insidon); Pipofezine (Azafen/Azaphen); Protriptyline (Vivactil); Trimipramine (Surmontil); Amoxapine (Asendin); Maprotiline (Ludiomil); Mianserin (Tolvon); Mirtazapine (Remeron); Setiptiline (Tecipul); Isocarboxazid (Marplan); Phenelzine (Nardil); Tranylcypromine (Parnate); Selegiline (Eldepryl, Zelapar, Emsam); Caroxazone (Surodil, Timostenil); Metralindole (Inkazan); Moclobemide (Aurorix, Manerix); Pirlindole (Pirazidol); Toloxatone (Humoryl); Eprobemide (Befol); minaprine (Brantur, Cantor); Bifemelane (Alnert, Celeport); Agomelatine (Valdoxan); Esketamine (Spravato); Ketamine (Ketalar); Tandospirone (Sediel); Tianeptine (Stablon, Coaxil); α-Methyltryptamine (Indopan); Etryptamine, α-Ethyltryptamine (αET), (Monase); Indeloxazine (Elen, Noin); Medifoxamine (Cledial, Gerdaxyl); Oxaflozane (Conflictan); Pivagabine (Tonerg); Ademetionine, S-Adenosyl-L-methionine (SAMe), (Heptral, Transmetil, Samyl); Hypericum perforatum (Jarsin, Kira, Movina); Oxitriptan, 5-Hydroxytryptophan (5-HTP), (Cincofarm, Levothym, Triptum); Rubidium chloride (Rubinorm); Tryptophan (Tryptan, Optimax, Aminomine); Magnesium; Acetylcarnitine; Amisulpride (Solian); Aripiprazole (Abilify); Brexpiprazole (Rexulti); Lurasidone (Latuda); Olanzapine (Zyprexa); Quetiapine (Seroquel); Risperidone (Risperdal); Buspirone (Buspar); Lithium (Eskalith, Lithobid); Modafinil; Thyroxine; Triiodothyronine; Minocycline; Amitriptyline, chlordiazepoxide; benzodiazepine (Limbitrol); Amitriptyline, perphenazine (Etafron); Flupentixol, melitracen (Deanxit); Olanzapine, fluoxetine (Symbyax); Tranylcypromine and trifluoperazine.

### 1.9 Catalyst

In certain embodiments, the composition further includes a catalyst that facilitates hydrosilylation reaction of the one or more crosslinkable polymers or bifunctional organopolysiloxane polymer. “Catalyst” includes any substance that causes, facilitates, or initiates a physical and/or chemical hydrosilylation reaction. The catalyst may or may not undergo permanent physical and/or chemical changes during or at the end of the process. In preferred embodiments, the catalyst is a metal catalyst capable of initiating and/or facilitating the hydrosilylation at or below body temperature, for example, Group VIII metal catalysts, such as platinum, rhodium, palladium, cobalt, nickel, ruthenium, osmium and iridium catalysts, and Group IVA metal catalysts, such as germanium and tin. In further preferred embodiments, the catalyst is a platinum catalyst, a rhodium catalyst or a tin catalyst. Examples of platinum catalysts include, for example, platinum carbonyl cyclovinylmethylsiloxane complexes, platinum divinyltetramethyldisiloxane complexes, platinum cyclovinylmethylsiloxane complexes, platinum octanaldehyde/octanol complexes, and other Pt(0) catalysts such as Karstedt’s catalyst, platinum-alcohol complexes, platinum-alkoxide complexes, platinum-ether complexes, platinum-aldehyde complexes, platinum-ketone complexes, platinum-halogen complexes, platinum-sulfur complexes, platinum-nitrogen complexes, platinum-phophorus complexes, platinum-carbon double-bond complexes, platinum carbon triple-bond complexes, platinum-imide complexes, platinum-amide complexes, platinum-ester complexes, platinum-phosphate ester complexes, platinum-thiol ester complexes, platinum lone-pair-electron complexes, platinum-aromatic complexes, platinum π-electron complexes, and combinations thereof. Examples of rhodium catalyst include tris (dibutylsulfide) rhodium trichloride and rhodium trichloride hydrate. Examples of tin catalysts include tin II octoate, tin II neodecanoate, dibutyltin diisooctylmaleate, di-n-butylbis(2,4 pentanedionate)tin, din-butylbutoxychlorotin, dibutyltin dilaurate, dimethyltin dineodecanoate, dimethylhydroxy(oleate)tin and tin II oleate. In preferred embodiments, the catalyst is platinum catalyst. In further preferred embodiments, the catalyst is platinum divinyltetramethyldisiloxane complexes.

In one embodiment, the catalyst is a transition metal. In one embodiment, the transition metal is platinum.

In preferred embodiments, the composition includes about 0.001 to about 1% by weight (i.e., about 10 ppm to about 1,000 ppm), preferably about 0.005 to about 0.05% by weight (i.e., about 50 ppm to about 500 ppm) catalyst. In further preferred embodiments, the composition includes about 0.01 to about 0.03% by weight catalyst.

In one embodiment, the molar ratio of transition metal to ligand is between about 10:1 to about 1:10000. In one embodiment, the molar ratio of transition metal to ligand is between about 1:250 to about 1:750. In one embodiment, the molar ratio of transition metal to ligand is about 1:500. In one embodiment, the vinyl to functional hydride molar ratio is between about 1:10 and about 1:100. In one embodiment, the vinyl to functional hydride molar ratio is between about 1:15 and about 1:90. In one embodiment, the vinyl to functional hydride molar ratio is between about 1:25 and about 1:70. In one embodiment, the vinyl to functional hydride molar ratio is between about 1:30 and about 1:60. In one embodiment, the composition has a viscosity of between about 5,000 and 700,000 cSt or cP at about 25° C. In one embodiment, the molar ratio of hydride functionalized polysiloxane to ligand is between about 10:1 to about 1:10000. In one embodiment, the molar ratio of hydride functionalized polysiloxane to ligand is between about 1:250 to about 1:750. In one embodiment, the molar ratio of hydride functionalized polysiloxane to ligand is about 1:500.

In one embodiment, the molar ratio of transition metal or hydride functionalized polysiloxane to encapsulating agent is between about 10:1 to about 1:10000. In one embodiment, the molar ratio of transition metal to encapsulating agent is between about 1:250 to about 1:750. In one embodiment, the molar ratio of transition metal to encapsulating agent is about 1:500. In one embodiment, the molar ratio of hydride functionalized polysiloxane to encapsulating agent is between about 1:250 to about 1:750. In one embodiment, the molar ratio of hydride functionalized polysiloxane to encapsulating agent is about 1:500.

### 1.10 Ligand-Catalyst Complex

In one embodiment, the ligand-catalyst complex is Karstedt’s catalyst. In one embodiment, the ligand-catalyst complex has the chemical formula of C₂₄H₅₄O₃Pt₂Si₆. In one embodiment, the ligand-catalyst complex has the following structure: [0277]

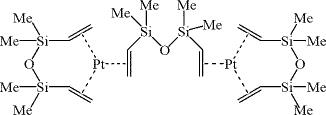

In one embodiment, the ligand in the ligand-catalyst complex is 1,3-divinyltetramethyldisiloxane. In one embodiment, the ligand has the chemical formula of C₈H₁₈OSi₂. In one embodiment, the ligand has the following structure:

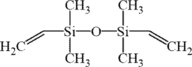

In one embodiment, the ligand in the ligand-catalyst complex is 1,1,3,3,5,5-hexamethyl-1,5-divinyltrisiloxane. In one embodiment, the ligand has the chemical formula of C₁₀H₂₄O₂Si₃. In one embodiment, the ligand has the following structure:

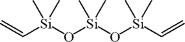

In one embodiment, the ligand in the ligand-catalyst complex is 1,5-divinyl-3-phenylpentamethyltrisiloxane. In one embodiment, the ligand has the chemical formula of C₁₅H₂₆O₂Si₃. In one embodiment, the ligand has the following structure:

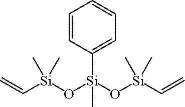 [SMILES: C=C[Si](C)(C)O[Si](C)(O[Si](C)(C)C=C)c1ccccc1]

In one embodiment, the ligand in the ligand-catalyst complex is 1,1,5,5-tetramethyl-3,3-diphenyl-1,5-divinyltrisiloxane. In one embodiment, the ligand has the chemical formula of C₂₀H₂₈O₂Si₃. In one embodiment, the ligand has the following structure:

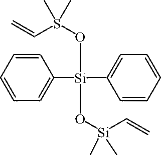 [SMILES: C=C[Si](C)(C)O[Si](OS(C)(C)C=C)(c1ccccc1)c1ccccc1]

In one embodiment, the ligand in the ligand-catalyst complex is 1,3,5-trivinyl-1,3,5-trimethylcyclotrisiloxane. In one embodiment, the ligand has the chemical formula of C₉H₁₈O₃Si₃. In one embodiment, the ligand has the following structure:

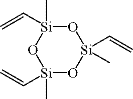

In one embodiment, the ligand in the ligand-catalyst complex is 2,4,6,8-tetramethyltetravinylcyclotetrasiloxane. In one embodiment, the ligand has the chemical formula of C₁₂H₂₄O₄Si₄. In one embodiment, the ligand has the following structure:

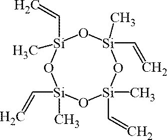 [SMILES: C=C[Si]1(C)O[Si](C)(C=C)O[Si](C)(C=C)O[Si](C)(C=C)O1]

In one embodiment, the ligand in the ligand-catalyst complex is 1,3,5,7,9-pentamethyl-1,3,5,7,9-pentavinylcyclopentasiloxane. In one embodiment, the ligand has the chemical formula of C₁₅H₃₀O₅Si₅. In one embodiment, the ligand has the following structure:

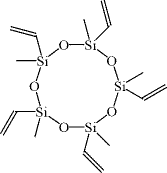 [SMILES: C=C[Si]1(C)O[Si](C)(C=C)O[Si](C)(C=C)O[Si](C)(C=C)O[Si](C)(C=C)O1]

In one embodiment, the ligand in the ligand-catalyst complex is tris(vinyldimethylsiloxy)methylsilane. In one embodiment, the ligand has the chemical formula of C₁₃H₃₀O₃Si₄. In one embodiment, the ligand has the following structure:

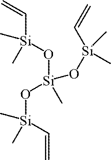

In one embodiment, the ligand in the ligand-catalyst complex is tetrakis(vinyldimethylsiloxy)silane. In one embodiment, the ligand has the chemical formula of C₁₆H₃₆O₄Si₅. In one embodiment, the ligand has the following structure:

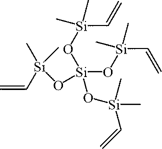 [SMILES: C=C[Si](C)(C)O[Si](O[Si](C)(C)C=C)(O[Si](C)(C)C=C)O[Si](C)(C)C=C]

In one embodiment, the ligand in the ligand-catalyst complex is methacryloxypropyltris(vinyldimethylsiloxy)silane. In one embodiment, the ligand has the chemical formula of C₁₉H₃₈O₅Si₄. In one embodiment, the ligand has the following structure:

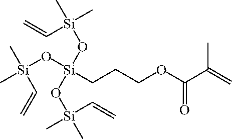 [SMILES: C=C[Si](C)(C)O[Si](CCCOC(=O)C(=C)C)(O[Si](C)(C)C=C)O[Si](C)(C)C=C]

In one embodiment, the ligand in the ligand-catalyst complex is 1,2-divinyltetramethyldisilane. In one embodiment, the ligand has the chemical formula of C₈H₁₈O₅Si₂. In one embodiment, the ligand has the following structure:

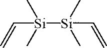

In one embodiment, the ligand in the ligand-catalyst complex is 1,5-hexadiene. In one embodiment, the ligand has the chemical formula of C₆H₁₀. In one embodiment, the ligand has the following structure:

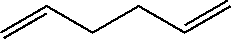

In one embodiment, the ligand in the ligand-catalyst complex is 1,4-hexadiene. In one embodiment, the ligand has the chemical formula of C₆H₁₀. In one embodiment, the ligand has the following structure:

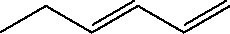

In one embodiment, the ligand in the ligand-catalyst complex is Octadiene. In one embodiment, the ligand has the chemical formula of C₈H₁₄. In one embodiment, the ligand has one of the following structures:

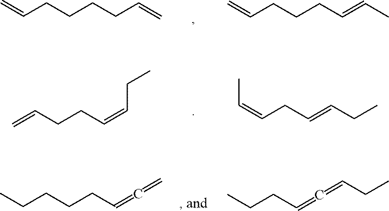

In one embodiment, the ligand in the ligand-catalyst complex is dimethylbutadiene. In one embodiment, the ligand has the chemical formula of C₆H₁₀. In one embodiment, the ligand has the following structure:

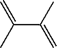

In one embodiment, the ligand in the ligand-catalyst complex is dimethylhexadiene. In one embodiment, the ligand has the chemical formula of C₈H₁₄. In one embodiment, the ligand has the following structure:

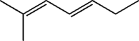

In one embodiment, the ligand in the ligand-catalyst complex is dimethyloctadiene. In one embodiment, the ligand has the chemical formula of C₁₀H₁₈. In one embodiment, the ligand has the following structure:

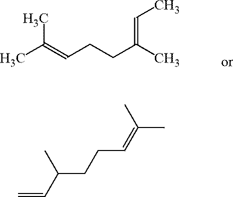

In one embodiment, the ligand in the ligand-catalyst complex is methyl vinyl ketone. In one embodiment, the ligand has the chemical formula of C₄H₆O. In one embodiment, the ligand has the following structure:

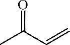

In one embodiment, the ligand in the ligand-catalyst complex is dimethyl maleate. In one embodiment, the ligand has the chemical formula of C₆H₈O₄. In one embodiment, the ligand has the following structure:

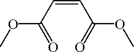

In one embodiment, the ligand in the ligand-catalyst complex is dimethyl fumarate. In one embodiment, the ligand has the chemical formula of C₆H₈O₄. In one embodiment, the ligand has the following structure:

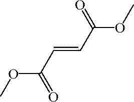

In one embodiment, the ligand in the ligand-catalyst complex is (3E)-4-methoxy-3-buten-2-one. In one embodiment, the ligand has the chemical formula of C₅H₈O₂. In one embodiment, the ligand has the following structure:

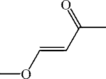

In one embodiment, the ligand in the ligand-catalyst complex is (E)-2-ethylhex-2-enal. In one embodiment, the ligand has the chemical formula of C₈H ₁₄O. In one embodiment, the ligand has the following structure:

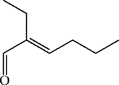

In one embodiment, the ligand in the ligand-catalyst complex is pent-1-en-3-one. In one embodiment, the ligand has the chemical formula of C₅H₈O. In one embodiment, the ligand has the following structure:

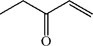

### 1.11 Encapsulating Agent-Catalyst Microcapsules

In one embodiment, the encapsulating agent-catalyst microcapsules are prepared by emulsion polymerization, suspension polymerization, interfacial polymerization, coacervation/phase separation, solvent evaporation/extraction, sol-gel encapsulation, supercritical fluid-assisted microencapsulation, layer-by-layer assembly, spray-drying, spray-cooling, co-extrusion, spinning disk, fluidized-bed coating, melt solidification, or polymer precipitation. In one embodiment, the encapsulating agent-catalyst microcapsules are prepared by solvent evaporation/extraction or spray-drying. In one embodiment, the encapsulating agent-catalyst microcapsules are prepared by solvent evaporation/extraction. In one embodiment, the encapsulating agent-catalyst microcapsules are prepared by spray-drying.

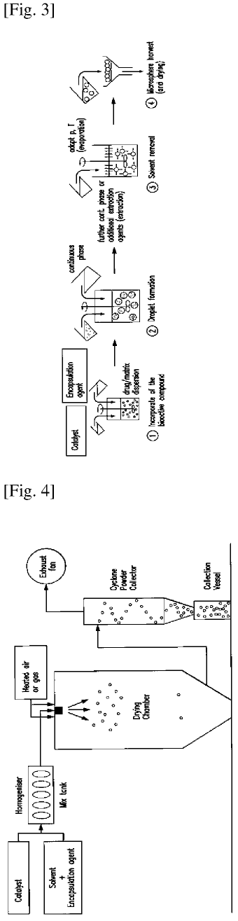

A schematic representation of the solvent evaporation process is presented in FIG. 3. In this method a water insoluble encapsulating agent is dissolved in a water immiscible volatile organic solvent, e.g., dichloromethane or chloroform or disiloxane or isododecane, into which the catalyst is also dissolved or dispersed. The resulting solution is added dropwise to a stirring aqueous solution having a suitable stabilizer to form small polymer droplets containing the encapsulated material. The core material may also be dispersed or dissolved in this aqueous solution instead. After a reasonable aging time, the droplets are hardened to produce the corresponding polymer microcapsules. This hardening process is accomplished by removal of the solvent from the polymer droplets either by solvent evaporation (by heat or reduced pressure), or by solvent extraction (with a third liquid which is a precipitant).

A schematic representation of the spray drying process is presented in FIG. 4. The catalyst to be encapsulated is added to the solvent (the ratio of catalyst to solvent may be optimized) and the mixture is homogenized. The encapsulating agent is added at this stage. This mixture is then fed into the spray dryer with circulating hot air and atomized, which can be made by different types of atomizers: pneumatic atomizer, pressure nozzle, spinning disk, fluid nozzle and sonic nozzle. The solvent is evaporated by hot air and the encapsulating agent encapsulates the catalyst. Small particles of the resulting microcapsules are deposited in the collection vessel where they are collected.

### 1.12 Single Formulation Organopolysiloxane Polymer For Use With The Compositions And Methods Provided Herein

Without being bound by theory, the ability of the ligand to reduce or prevent the activity of the catalyst to cross-link the unsaturated organopolymer or vinyl functionalized organopolysiloxane and the hydride functionalized polysiloxane makes it possible to formulate the various components into a single formulation without cross-linking and polymer-formation prior to the application of the formulation, e.g., by applying the formulation to the skin of a subject. Without being bound by theory, the ability of the encapsulating agent to reduce or prevent the activity of the catalyst to cross-link the unsaturated organopolymer or vinyl functionalized organopolysiloxane and the hydride functionalized polysiloxane, or to reduce or prevent the activity of hydride functionalized polysiloxane to react with the unsaturated organopolymer or vinyl functionalized organopolysiloxane as facilitated by catalyst, makes it possible to formulate the various components into a single formulation without cross-linking and polymer-formation prior to the application of the formulation, e.g., by applying the formulation to the skin of a subject.

Provided herein is a single formulation that enables one-step room temperature vulcanizing (RTV). In one embodiment, the formulation provided herein is capable of vulcanizing at room temperature in one-step. In one embodiment, the formulation provided herein is capable of vulcanizing at room temperature in one-step, without the need to a priori separate into formulations containing hydride functional groups and the catalyst individually.

### 1.13 Reinforcing Constituents For Use With The Methods Provided Herein

In preferred embodiments, a composition provided herein further includes one or more reinforcing constituent(s). In certain embodiments, the reinforcing constituent is selected from surface treated carbon, graphene, diamond, carbon nanotube, graphene oxide, silver, mica, zinc sulfide, zinc oxide, titanium dioxide, aluminum oxide, clay (e.g., Al₂O₃, SiO₂), chalk, talc, calcite (e.g., CaCO₃), barium sulfate, zirconium dioxide, polymer beads and silica (e.g., silica aluminates, calcium silicates, or surface treated silica (e.g., fumed silica, hydrated silica, or anhydrous silica)), or a combination thereof. Such reinforcing constituents reinforce the physical properties of the layer as discussed herein. In preferred embodiments, the reinforcing constituent is surface treated silica, for example, silica treated with hexamethyldisilazane, polydimethylsiloxane, hexadecylsilane or methacrylsilane. In further preferred embodiments, the reinforcing constituent is fumed silica, including fumed silica having been surface treated with hexamethyldisilazane. In further preferred embodiments, the reinforcing constituent includes nanofibers.

In certain embodiments, the particles of the reinforcing constituent have an average surface area of between about 50 and about 1000 m²/g. In certain embodiments, the particles of the reinforcing constituent have an average surface area of between about 50 and about 500 m²/g. In preferred embodiments, the particles of the reinforcing constituent have an average surface area of between about 100 and about 350 m²/g. In further preferred embodiments, the particles of the reinforcing constituent have an average surface area of between about 135 and about 250 m²/g. In certain embodiments, the reinforcing constituent has an average particle diameter of between about 1 nm and about 20 µm. In preferred embodiments, the reinforcing constituent has an average particle diameter of between about 2 nm and about 1 µm, and further preferably between about 5 nm and about 50 nm.

### 1.14 Optional Additional Agents

In some embodiments, the film is used in combination with one or more additional therapeutic agents. In some embodiments, the additional therapeutic agent is a moisturizer, mineral oil, petroleum jelly, coal tar, anthralin, corticosteroids, fluocinonide, vitamin D3 analogues, retinoids, phototherapy, methotrexate, cyclosporine, a monoclonal antibody, pimecrolimus, tacrolimus, azathioprine, fluoruracil, salicylic acid, benzoyl peroxide, antibiotics or alpha-hydroxy acids.

### 1.15 Additives For Use With The Compositions And Methods Provided Herein

In certain embodiments, the composition further includes one or more additives. In certain embodiments, the composition provided herein further independently include(s) one or more additives. Suitable additives include, but are not limited to, feel modifiers, tack modifiers, spreadability enhancers, diluents, adhesion modifiers, volatile siloxanes, emulsifiers, emollients, surfactants, lubricants, thickeners, solvents, film formers, humectants, preservatives, pigments, skin permeation enhancers, optic modifiers, gas transport modifiers, liquid transport modifiers, pH modifiers, sensitizing modifiers, aesthetic modifiers, and a combination thereof. Additional suitable additives are disclosed in the International Nomenclature Cosmetic Ingredient (INCI) dictionary, which is incorporated herein by reference in its entirety. In preferred embodiments, the emulsifiers are alkoxydimethicone, alkyldimethicone, amodimethicone, sulfodimethicone, phosphodimethicone, borodimethicone, halodimethicone, fluorodimethicone, chlorodimethicone, bromodimethicone, charged dimethicone, and a combination thereof. In preferred embodiments, the emulsifiers are of linear-type, branch-type, elastomeric-type network, elastomeric-type organic/inorganic network, and a combination thereof.

In preferred embodiments, the composition further includes one or more optics modifiers. In other preferred embodiments, the first part and/or the second part further independently include one or more optics modifiers or particles. Optics modifiers or particles introduce surfaces responsive to optical or photonic interaction, e.g., roughness for light scattering, thereby imparting desirable shine, glossy, glow, matte appearance beyond or comparable to that of normal, healthy skin, preferably avoiding a significantly more shiny and/or glossy appearance than normal skin. Suitable optics modifiers or particles include, for example, pigments, dyes, polymers such as nylon (e.g., nylon-6, nylon-10, nylon-12), silicone, acrylic, acrylates/carbamate or other polymer or copolymer beads or particles, polyethylene beads, polymethymethacrylate beads, polystyrene beads, polyurethane beads; inorganics such as silica (e.g., silica and DMPA/isophthalic acid/SMDI copolymer, available as ChronoSphere (Registered Trademark) Opticals from Lonza Group), boron nitride, talc, mica, alumina, titania; metal such as silver nanoparticles; and silicone, acrylic, acrylates/carbamate or other polymer or copolymer beads or particles. In certain embodiments, the optics modifiers or particles have an average particle diameter of between about 1 µm and about 20 µm. In a preferred embodiment, the optics modifiers or particles have an average particle diameter of between about 0.1 µm and about 20 µm. In preferred embodiments, the optics modifiers or particles have an average particle diameter of 2 µm to 15 µm, and further preferably 5 to 10 µm.

In certain embodiments, the composition further includes one or more additional agents. In certain embodiments, the composition provided herein further independently include(s) one or more additional agents, including cosmetic agents, therapeutic agents, stimuli-responsive agents, sensing agents, drug-delivery agents, optical agents, coloring agents, pigments, scattering agents, sorbing agents, temperature-active agents, heat-active agents, UV-active agents, light-active agents, sound-active agents, pressure-active agents, motion-active agents, radioactive agents, electrical agents, and magnetic agents.

### 1.16 Methods Of Using

Provided herein are methods for use of the compositions provided herein for cosmetic applications.

In one embodiment, a method for transporting an environment-responsive agent out or facilitating out-transport of agents from the film formed by the composition provided here, includes applying the composition to a subject. In one embodiment, a method for transporting an environment-responsive agent out or facilitating out-transport of agents from the film formed by the composition provided here, includes applying the composition to a subject, wherein the agent is transported out in a controlled manner. In one embodiment, a method for transporting an environment-responsive agent out or facilitating out-transport of agents from the film formed by the composition provided here, includes applying the composition to a subject, wherein the agent is transported out of the composition by convection. In one embodiment, a method for transporting an environment-responsive agent out or facilitating out-transport of agents from the film formed by the composition provided here, includes applying the composition to a subject, wherein the agent is transported out of the composition by diffusion.

In one embodiment, a method for transporting an environment-responsive agent out or facilitating out-transport of agents from the film formed by the composition provided here, includes applying the composition to a subject, wherein the agent is transferred out of the composition provided herein by evaporation. In one embodiment, a method for transporting an environment-responsive agent out or facilitating out-transport of agents from the film formed by the composition provided here, includes applying the composition to a subject, wherein the agent is transferred out of the composition provided herein by applying pressure to the film. In one embodiment, a method for transporting an environment-responsive agent out or facilitating out-transport of agents from the film formed by the composition provided here, includes applying the composition to a subject, wherein the agent is transferred out of the composition provided herein by exposure to a sound, chemical, heat or light. In one embodiment, a method for transporting an environment-responsive agent out or facilitating out-transport of agents from the film formed by the composition provided here, includes applying the composition to a subject, wherein the agent is transferred out of the composition provided herein by absorbing the environment-responsive agent into another phase. In one embodiment, a method for transporting an environment-responsive agent out or facilitating out-transport of agents from the film formed by the composition provided here, includes applying the composition to a subject, wherein the agent is transferred out of the composition provided herein by absorbing the environment-responsive agent into the skin of a subject. In one embodiment, a method for transporting an environment-responsive agent out or facilitating out-transport of agents from the film formed by the composition provided here, includes applying the composition to a subject, wherein the agent is transferred out of the composition provided herein by absorbing the environment-responsive agent into another ingredient forming a complex. In one embodiment, a method for transporting an environment-responsive agent out or facilitating out-transport of agents from the film formed by the composition provided here, includes applying the composition to a subject, wherein the agent is transferred out of the composition provided herein by using heat. In one embodiment, a method for transporting an environment-responsive agent out or facilitating out-transport of agents from the film formed by the composition provided here, includes applying the composition to a subject, wherein the agent is transferred out of the composition provided herein by cooling the composition. In one embodiment, a method for transporting an environment-responsive agent out or facilitating out-transport of agents from the film formed by the composition provided here, includes applying the composition to a subject, wherein the agent is transferred out of the composition provided herein by using heat generated with a blow-dry. In one embodiment, a method for transporting an environment-responsive agent out or facilitating out-transport of agents from the film formed by the composition provided here, includes applying the composition to a subject, wherein the agent is transferred out of the composition provided herein by using ultrasound. In one embodiment, a method for transporting an environment-responsive agent out or facilitating out-transport of agents from the film formed by the composition provided here, includes applying the composition to a subject, wherein the agent is transferred out of the composition provided herein by exposure to radiative transfer of electomagetic waves. In one embodiment, a method for transporting an environment-responsive agent out or facilitating out-transport of agents from the film formed by the composition provided here, includes applying the composition to a subject, wherein the agent is transferred out of the composition provided herein by using electromagnetic waves. In one embodiment, a method for transporting an environment-responsive agent out or facilitating out-transport of agents from the film formed by the composition provided here, includes applying the composition to a subject, wherein the agent is transferred out of the composition provided herein by using visible light. In one embodiment, a method for transporting an environment-responsive agent out or facilitating out-transport of agents from the film formed by the composition provided here, includes applying the composition to a subject, wherein the agent is transferred out of the composition provided herein by using ultraviolet light. In one embodiment, a method for transporting an environment-responsive agent out or facilitating out-transport of agents from the film formed by the composition provided here, includes applying the composition to a subject, wherein the agent is transferred out of the composition provided herein by using infrared radiation.

In one embodiment, a method for transporting an environment-responsive agent out or facilitating out-transport of agents from the film formed by the composition provided here, includes applying the composition to a subject, wherein the environment-responsive agent is fragrance, a volatile active, a drug, a beneficial agent, an active that attracts animals, an active that repels animals, a pheromone, an insect repellent, a mosquito repellent, a cooling agent, a heating agent, an antihistamine, an odor masking active, a humidity control agent, an inhalant, an antidepressant, or nicotine.

In one embodiment, a method for transporting a fragrance out from the film formed by the composition provided here, includes applying the composition to a subject, wherein the volatile agent is the fragrance. In one embodiment, a method for transporting a skincare out from the film formed by the composition provided here, includes applying the composition to a subject, wherein the volatile agent is for personal care. In one embodiment, a method for transporting a skincare out from the film formed by the composition provided here, includes applying the composition to a subject, wherein the volatile agent is for skincare, haircare, or bodycare. In one embodiment, a method for transporting a skincare out from the film formed by the composition provided here, includes applying the composition to a subject, wherein the volatile agent is for skincare.

Provided herein are methods for use of the compositions provided herein for cosmeceutical applications. In one embodiment, a method for transporting an essential oil out from the film formed by the composition provided here, includes applying the composition to a subject, wherein the volatile agent is the essential oil.

Provided herein are methods for use of the compositions provided herein for therapeutic applications. In one embodiment, a method for transporting an inhalant out from the film formed by the composition provided here, includes applying the composition to a subject, wherein the volatile agent is inhalant. In one embodiment, the inhalant is useful for treatment of anastasia. In one embodiment, the inhalant is useful for treatment of allergy. In one embodiment, the inhalant is useful for treatment of respiratory system. In one embodiment, the inhalant is useful for treatment of pulmonary. In one embodiment, the inhalant is useful for treatment inside nasal cavity. In one embodiment, the inhalant is useful for treatment of oral pathway.

Provided herein is a method of using a composition provided herein as a single formulation in a one-step method without the need to separate the hydride and the catalyst complex from each other before application to the skin of a subject.

Provided herein is a method of using a composition provided herein to form a thin film on the skin of a subject. In certain embodiments, such a method includes separating the ligand from the catalyst (e.g., transition metal) or from the hydride functionalized polysiloxane in a composition provided herein. Without being limited by theory, separating the ligand from the catalyst (e.g., transition metal) or from the hydride functionalized polysiloxane accelerates the hydrosilylation reaction. In certain embodiments, the separating step involves evaporating the ligand, absorbing the ligand into another phase, absorbing the ligand into the skin of a subject, absorbing the ligand into another ingredients forming a complex, transforming the ligand into non-complex with the transition metal or with the hydride functionalized polysiloxane, heating the composition, cooling the composition, applying ultrasound on the composition, applying electromagnetic waves on the composition, applying visible light on the composition, applying ultraviolet light on the composition, or applying infrared radiation on the composition. Provided herein is a method of using a composition provided herein as a single formulation in a one-step method, including separating at least one divinyl disiloxane from platinum in a composition provided herein, such as a composition that includes (a) the platinum; (b) at least one vinyl functionalized organopolysiloxane; (c) at least one hydride functionalized polysiloxane; (d) a volatile agent that is capable of transporting out of the composition or facilitate out-transport of one or more beneficial agents from the composition after the composition is applied to a subject and (e) the divinyl disiloxane at a concentration sufficient to slow down hydrosilylation reaction between the vinyl functionalized organopolysiloxane and the hydride functionalized polysiloxane, such that these components can be formulated and stored together as a mixture without significant cross-linking. In one embodiment, the method includes separating the ligand from the transition metal or from the hydride functionalized polysiloxane by evaporating the ligand with or without using heat.

Provided herein is a method of using a composition provided herein to form a thin film on the skin of a subject. In certain embodiments, such a method includes separating the encapsulating agent from the catalyst (e.g., transition metal) or from the hydride functionalized polysiloxane in a composition provided herein. Without being limited by theory, separating the encapsulating agent from the catalyst (e.g., transition metal) accelerates the cross-linking reaction or separating the encapsulating agent from the hydride functionalized polysiloxane enables the cross-linking reaction. In certain embodiments, the separating step involves evaporating the encapsulating agent, absorbing the encapsulating agent into another phase, absorbing the encapsulating agent into the skin of a subject, absorbing the encapsulating agent into another ingredients forming a complex, transforming the encapsulating agent into non-microcapsule with the transition metal or with the hydride functionalized polysiloxane, heating the composition, cooling the composition, applying ultrasound on the composition, applying electromagnetic waves on the composition, applying visible light on the composition, applying ultraviolet light on the composition, or applying infrared radiation on the composition. Provided herein is a method of using a composition provided herein as a single formulation in a one-step method, including separating at least one polyurethane-1 from platinum or from the hydride functionalized polysiloxane in a composition provided herein, such as a composition that includes (a) the platinum; (b) at least one vinyl functionalized organopolysiloxane; (c) at least one hydride functionalized polysiloxane; (d) a volatile agent that is capable of transporting out of the composition or facilitate out-transport of one or more beneficial agents from the composition after the composition is applied to a subject and (e) the polyurethane-1 at a concentration sufficient to slow down or prohibit the cross-linking reaction between the vinyl functionalized organopolysiloxane and the hydride functionalized polysiloxane, such that these components can be formulated and stored together as a mixture without significant cross-linking. In one embodiment, the method includes separating the encapsulating agent from the transition metal or from the hydride functionalized polysiloxane by evaporating the encapsulating agent with or without using heat.

In one embodiment, provided herein is a method for treating symptoms of conditions of compromised skin barrier function with the formulations and films disclosed herein. In one aspect of this embodiment, the invention provides formulations, film and methods for treating itchy skin; for treating raw skin; for treating dry skin; for treating flaking or peeling skin; for treating blisters on skin; for treating redness or swelling or inflammation of the skin; or for treating oozing, scabbing and scaling skin.

In one embodiment, provided herein is a method for occluding skin on a subject in need thereof, including: applying to the subject a composition provided herein including a catalyst; at least one environment-responsive agent; at least one ligand; at least one unsaturated organopolymer; and at least one hydride functionalized polysiloxane, wherein said catalyst facilitates in situ cross-linking of the at least one unsaturated organopolymer; and at least one hydride functionalized polysiloxane, such that a film is formed on skin, thereby occluding the skin. In one embodiment, provided herein is a method for occluding skin on a subject in need thereof, including: applying to the subject a composition provided herein including a catalyst; at least one volatile agent; at least one ligand; at least one vinyl functionalized organopolysiloxane; and at least one hydride functionalized polysiloxane, wherein said catalyst facilitates in situ cross-linking of the at least one vinyl functionalized organopolysiloxane; and at least one hydride functionalized polysiloxane, such that a film is formed on skin, thereby occluding the skin. In one embodiment, provided herein is a method for occluding skin on a subject in need thereof, including: applying to the subject a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin, thereby occluding the skin. In one embodiment, provided herein is a method for occluding skin on a subject in need thereof, including: applying to the subject a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin, thereby occluding the skin.

In one embodiment, provided herein is a method for occluding skin on a subject in need thereof, including: applying to the subject a composition provided herein including a catalyst; at least one environment-responsive agent; at least one encapsulating agent; at least one unsaturated organopolymer; and at least one hydride functionalized polysiloxane, wherein said catalyst facilitates in situ cross-linking of the at least one unsaturated organopolymer; and at least one hydride functionalized polysiloxane, such that a film is formed on skin, thereby occluding the skin. In one embodiment, provided herein is a method for occluding skin on a subject in need thereof, including: applying to the subject a composition provided herein including a catalyst; at least one volatile agent; at least one encapsulating agent; at least one vinyl functionalized organopolysiloxane; and at least one hydride functionalized polysiloxane, wherein said catalyst facilitates in situ cross-linking of the at least one vinyl functionalized organopolysiloxane; and at least one hydride functionalized polysiloxane, such that a film is formed on skin, thereby occluding the skin. In one embodiment, provided herein is a method for occluding skin on a subject in need thereof, including: applying to the subject a composition provided herein including a catalyst; at least one environment-responsive agent; at least one encapsulating agent; and at least one bifunctional organopolysiloxane polymer, wherein said catalyst facilitates in situ hydrosilylation step-growth polymerization reaction of the bifunctional organopolysiloxane polymer, such that a film is formed on skin, thereby occluding the skin. In one embodiment, provided herein is a method for occluding skin on a subject in need thereof, including: applying to the subject a composition provided herein including a catalyst; at least one volatile agent; at least one encapsulating agent; and at least one bifunctional organopolysiloxane polymer, wherein said catalyst facilitates in situ hydrosilylation step-growth polymerization reaction of the bifunctional organopolysiloxane polymer, such that a film is formed on skin, thereby occluding the skin.

In one embodiment, provided herein is a method for hydrating skin in a subject in need thereof, including: applying to the subject’s skin a composition provided herein including a catalyst; at least one ligand; at least one environment-responsive agent; at least one unsaturated organopolymer; and at least one hydride functionalized polysiloxane; wherein said catalyst facilitates in situ cross-linking of the at least one unsaturated organopolymer; and at least one hydride functionalized polysiloxane, such that a film is formed on skin, thereby hydrating the skin. In one embodiment, provided herein is a method for hydrating skin in a subject in need thereof, including: applying to the subject’s skin a composition provided herein including a catalyst; at least one ligand; at least one volatile agent; at least one vinyl functionalized organopolysiloxane; and at least one hydride functionalized polysiloxane; wherein said catalyst facilitates in situ cross-linking of the at least one vinyl functionalized organopolysiloxane; and at least one hydride functionalized polysiloxane, such that a film is formed on skin, thereby hydrating the skin. In one embodiment, provided herein is a method for hydrating skin in a subject in need thereof, including: applying to the subject’s skin a composition provided herein including a catalyst; at least one ligand; at least one environment-responsive agent; and at least one bifunctional organopolysiloxane polymer, wherein said catalyst facilitates in situ hydrosilylation step-growth polymerization reaction of the bifunctional organopolysiloxane polymer, such that a film is formed on skin, thereby hydrating the skin. In one embodiment, provided herein is a method for hydrating skin in a subject in need thereof, including: applying to the subject’s skin a composition provided herein including a catalyst; at least one ligand; at least one volatile agent; and at least one bifunctional organopolysiloxane polymer, wherein said catalyst facilitates in situ hydrosilylation step-growth polymerization reaction of the bifunctional organopolysiloxane polymer, such that a film is formed on skin, thereby hydrating the skin.

In one embodiment, provided herein is a method for hydrating skin in a subject in need thereof, including: applying to the subject’s skin a composition provided herein including a catalyst; at least one environment-responsive agent; at least one encapsulating agent; at least one unsaturated organopolymer; and at least one hydride functionalized polysiloxane; wherein said catalyst facilitates in situ cross-linking of the at least one unsaturated organopolymer; and at least one hydride functionalized polysiloxane, such that a film is formed on skin, thereby hydrating the skin. In one embodiment, provided herein is a method for hydrating skin in a subject in need thereof, including: applying to the subject’s skin a composition provided herein including a catalyst; at least one volatile agent; at least one encapsulating agent; at least one vinyl functionalized organopolysiloxane; and at least one hydride functionalized polysiloxane; wherein said catalyst facilitates in situ cross-linking of the at least one vinyl functionalized organopolysiloxane; and at least one hydride functionalized polysiloxane, such that a film is formed on skin, thereby hydrating the skin. In one embodiment, provided herein is a method for hydrating skin in a subject in need thereof, including: applying to the subject’s skin a composition provided herein including a catalyst; at least one environment-responsive agent; at least one encapsulating agent; and at least one bifunctional organopolysiloxane polymer; wherein said catalyst facilitates in situ hydrosilylation step-growth polymerization reaction of the bifunctional organopolysiloxane polymer, such that a film is formed on skin, thereby hydrating the skin. In one embodiment, provided herein is a method for hydrating skin in a subject in need thereof, including: applying to the subject’s skin a composition provided herein including a catalyst; at least one volatile agent; at least one encapsulating agent; and at least one bifunctional organopolysiloxane polymer; wherein said catalyst facilitates in situ hydrosilylation step-growth polymerization reaction of the bifunctional organopolysiloxane polymer, such that a film is formed on skin, thereby hydrating the skin.

In one embodiment, provided herein are methods for delivering out an agent from a subject, including applying to the subject a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, thereby delivering out the agent from the subject. In one embodiment, provided herein are methods for delivering out an agent from a subject, including applying to the subject a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane such that a film is formed on skin, thereby delivering out the agent from the subject. In one embodiment, provided herein are methods for delivering out an agent from a subject, including applying to the subject a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, thereby delivering out the agent from the subject. In one embodiment, provided herein are methods for delivering out an agent from a subject, including applying to the subject a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane such that a film is formed on skin, thereby delivering out the agent from the subject. In one embodiment, provided herein are methods for delivering out an agent from a subject, including applying to the subject a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer such that a film is formed on skin, thereby delivering out the agent from the subject. In one embodiment, provided herein are methods for delivering out an agent from a subject, including applying to the subject a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer such that a film is formed on skin, thereby delivering out the agent from the subject. In one embodiment, provided herein are methods for delivering out an agent from a subject, including applying to the subject a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer such that a film is formed on skin, thereby delivering out the agent from the subject. In one embodiment, provided herein are methods for delivering out an agent from a subject, including applying to the subject a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer such that a film is formed on skin, thereby delivering out the agent from the subject.

In another embodiment, provided herein is a film removing cleanser including a film wetting component, a penetration component, a film swelling component and a film release component.

In one embodiment, provided herein are methods for treating a subject post-laser treatment, including applying to the subject a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, thereby treating a subject post-laser treatment. In some embodiments, provided herein are methods for treating a subject post-laser treatment, including applying to the subject a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin, thereby treating a subject post-laser treatment. In one embodiment, provided herein are methods for treating a subject post-laser treatment, including applying to the subject a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, thereby treating a subject post-laser treatment. In some embodiments, provided herein are methods for treating a subject post-laser treatment, including applying to the subject a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin, thereby treating a subject post-laser treatment. In one embodiment, provided herein are methods for treating a subject post-laser treatment, including applying to the subject a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer such that a film is formed on skin, thereby treating a subject post-laser treatment. In some embodiments, provided herein are methods for treating a subject post-laser treatment, including applying to the subject a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject post-laser treatment. In one embodiment, provided herein are methods for treating a subject post-laser treatment, including applying to the subject a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer such that a film is formed on skin, thereby treating a subject post-laser treatment. In some embodiments, provided herein are methods for treating a subject post-laser treatment, including applying to the subject a composition provided herein including a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject post-laser treatment.

In some embodiments, provided herein are non-invasive formulations that form a film upon application to a subject post laser treatment, thereby facilitating healing of the subject post-laser treatment. In some embodiments, provided herein are methods of using such formulations. In some embodiments, provided herein are cleansers to remove the film.

In one embodiment, provided herein are methods for treating a subject post-laser treatment, including applying to the subject a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, such that a film is formed on skin. In some embodiments, provided herein is a composition for treating a subject post-laser treatment, wherein a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane upon application to skin, such that a film is formed on skin. In one embodiment, provided herein are methods for treating a subject post-laser treatment, including applying to the subject a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, such that a film is formed on skin. In some embodiments, provided herein is a composition for treating a subject post-laser treatment, wherein a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane upon application to skin, such that a film is formed on skin. In one embodiment, provided herein are methods for treating a subject post-laser treatment, including applying to the subject a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer such that a film is formed on skin, such that a film is formed on skin. In some embodiments, provided herein is a composition for treating a subject post-laser treatment, wherein a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer upon application to skin, such that a film is formed on skin. In one embodiment, provided herein are methods for treating a subject post-laser treatment, including applying to the subject a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer such that a film is formed on skin, such that a film is formed on skin. In some embodiments, provided herein is a composition for treating a subject post-laser treatment, wherein a composition provided herein including a catalyst; at least one volatile agent; at least one encapsulating agent; at least bifunctional organopolysiloxane polymer; in which the catalyst facilitates in situ hydrosilylation step-growth polymerization reaction of the bifunctional organopolysiloxane polymer upon application to skin, such that a film is formed on skin.

In some embodiments, provided herein are formulations for application to a subject post-laser treatment that including a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin and the film has an appearance of natural skin. In some embodiments, provided herein are formulations for application to a subject post-laser treatment that including a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin and the film has an appearance of natural skin. In some embodiments, provided herein are formulations for application to a subject post-laser treatment that including a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin and the film has an appearance of natural skin. In some embodiments, provided herein are formulations for application to a subject post-laser treatment that including a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin and the film has an appearance of natural skin. In some embodiments, provided herein are formulations for application to a subject post-laser treatment that including a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin. In some embodiments, provided herein are formulations for application to a subject post-laser treatment that including a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin. In some embodiments, provided herein are formulations for application to a subject post-laser treatment that including a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin. In some embodiments, provided herein are formulations for application to a subject post-laser treatment that including a composition provided herein including a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin.

In some embodiments, provided herein is a kit for use in treating a post-laser treatment on a subject in need thereof with a composition provided herein including a catalyst; at least one environment-responsive agent; at least one ligand; at least one unsaturated organopolymer; and at least one hydride functionalized polysiloxane; and instructions for use. In some embodiments, provided herein is a kit for use in treating a post-laser treatment on a subject in need thereof with a composition provided herein including a catalyst; at least one volatile agent; at least one ligand; at least one vinyl functionalized organopolysiloxane; and at least one hydride functionalized polysiloxane; and instructions for use. In some embodiments, provided herein is a kit for use in treating a post-laser treatment on a subject in need thereof with a composition provided herein including a catalyst; at least one environment-responsive agent; at least one encapsulating agent; at least one unsaturated organopolymer; and at least one hydride functionalized polysiloxane; and instructions for use. In some embodiments, provided herein is a kit for use in treating a post-laser treatment on a subject in need thereof with a composition provided herein including a catalyst; at least one volatile agent; at least one encapsulating agent; at least one vinyl functionalized organopolysiloxane; and at least one hydride functionalized polysiloxane; and instructions for use. In some embodiments, provided herein is a kit for use in treating a post-laser treatment on a subject in need thereof with a composition provided herein including a catalyst; at least one environment-responsive agent; at least one ligand; at least one bifunctional organopolysiloxane polymer; and instructions for use. In some embodiments, provided herein is a kit for use in treating a post-laser treatment on a subject in need thereof with a composition provided herein including a catalyst; at least one volatile agent; at least one ligand; at least one bifunctional organopolysiloxane polymer; and instructions for use. In some embodiments, provided herein is a kit for use in treating a post-laser treatment on a subject in need thereof with a composition provided herein including a catalyst; at least one environment-responsive agent; at least one encapsulating agent; at least one bifunctional organopolysiloxane polymer; and instructions for use. In some embodiments, provided herein is a kit for use in treating a post-laser treatment on a subject in need thereof with a composition provided herein including a catalyst; at least one volatile agent; at least one encapsulating agent; at least one bifunctional organopolysiloxane polymer; and instructions for use.

In some embodiments, provided herein are therapeutic formulations for application to a subject post-laser treatment, including at least one preselected function modulating component, in which the composition forms a therapeutic film upon application to the subject.

In some embodiments, provided herein are therapeutic formulations for application to a subject post-laser treatment on the subject that target a treatment area on a subject, wherein the targeted area includes an area that has been at least partially laser-treated, including at least one preselected treatment specific component, wherein the composition forms a therapeutic film upon application to the target treatment area on the subject.

In some embodiments, provided herein is a film removing cleanser for use in removing a therapeutic film used for post-laser treatment recovery management, wherein the film is prepared by a process including the steps of applying a composition provided herein including a catalyst; at least one environment-responsive agent; at least one ligand; at least one unsaturated organopolymer; and at least one hydride functionalized polysiloxane, and wherein said catalyst facilitates in situ cross-linking of the at least one unsaturated organopolymer; and at least one hydride functionalized polysiloxane. In some embodiments, provided herein is a film removing cleanser for use in removing a therapeutic film used for post-laser treatment recovery management, wherein the film is prepared by a process including the steps of applying a composition provided herein including a catalyst; at least one volatile agent; at least one ligand; at least one vinyl functionalized organopolysiloxane; and at least one hydride functionalized polysiloxane, and wherein said catalyst facilitates in situ cross-linking of the at least one vinyl functionalized organopolysiloxane; and at least one hydride functionalized polysiloxane. In some embodiments, provided herein is a film removing cleanser for use in removing a therapeutic film used for post-laser treatment recovery management, wherein the film is prepared by a process including the steps of applying a composition provided herein including a catalyst; at least one environment-responsive agent; at least one encapsulating agent; at least one unsaturated organopolymer; and at least one hydride functionalized polysiloxane, and wherein said catalyst facilitates in situ cross-linking of the at least one unsaturated organopolymer; and at least one hydride functionalized polysiloxane. In some embodiments, provided herein is a film removing cleanser for use in removing a therapeutic film used for post-laser treatment recovery management, wherein the film is prepared by a process including the steps of applying a composition provided herein including a catalyst; at least one volatile agent; at least one encapsulating agent; at least one vinyl functionalized organopolysiloxane; and at least one hydride functionalized polysiloxane, and wherein said catalyst facilitates in situ cross-linking of the at least one vinyl functionalized organopolysiloxane; and at least one hydride functionalized polysiloxane. In some embodiments, provided herein is a film removing cleanser for use in removing a therapeutic film used for post-laser treatment recovery management, wherein the film is prepared by a process including the steps of applying a composition provided herein including a catalyst; at least one environment-responsive agent; at least one ligand; at least one bifunctional organopolysiloxane polymer, and wherein said catalyst facilitates in situ hydrosilylation step-growth polymerization reaction of the bifunctional organopolysiloxane polymer. In some embodiments, provided herein is a film removing cleanser for use in removing a therapeutic film used for post-laser treatment recovery management, wherein the film is prepared by a process including the steps of applying a composition provided herein including a catalyst; at least one volatile agent; at least one ligand; at least one bifunctional organopolysiloxane polymer, and wherein said catalyst facilitates in situ hydrosilylation step-growth polymerization reaction of the bifunctional organopolysiloxane polymer. In some embodiments, provided herein is a film removing cleanser for use in removing a therapeutic film used for post-laser treatment recovery management, wherein the film is prepared by a process including the steps of applying a composition provided herein including a catalyst; at least one environment-responsive agent; at least one encapsulating agent; at least one bifunctional organopolysiloxane polymer, and wherein said catalyst facilitates in situ hydrosilylation step-growth polymerization reaction of the bifunctional organopolysiloxane polymer. In some embodiments, provided herein is a film removing cleanser for use in removing a therapeutic film used for post-laser treatment recovery management, wherein the film is prepared by a process including the steps of applying a composition provided herein including a catalyst; at least one volatile agent; at least one encapsulating agent; at least one bifunctional organopolysiloxane polymer, and wherein said catalyst facilitates in situ hydrosilylation step-growth polymerization reaction of the bifunctional organopolysiloxane polymer.

In some embodiments, provided herein is a film removing cleanser including a film wetting component, a penetration component, a film swelling component and a film release component.

In some embodiments, provided herein is a formulation for repairing a therapeutic film applied to a subject post-laser treatment, wherein said formulation includes a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a formulation for repairing a therapeutic film applied to a subject post-laser treatment, wherein said formulation includes a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a formulation for repairing a therapeutic film applied to a subject post-laser treatment, wherein said formulation includes a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a formulation for repairing a therapeutic film applied to a subject post-laser treatment, wherein said formulation includes a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a formulation for repairing a therapeutic film applied to a subject post-laser treatment, wherein said formulation includes a composition provided herein including a catalyst; at least one environment-responsive agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a formulation for repairing a therapeutic film applied to a subject post-laser treatment, wherein said formulation includes a composition provided herein including a catalyst; at least one volatile agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a formulation for repairing a therapeutic film applied to a subject post-laser treatment, wherein said formulation includes a composition provided herein including a catalyst; at least one environment-responsive agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a formulation for repairing a therapeutic film applied to a subject post-laser treatment, wherein said formulation includes a composition provided herein including a catalyst; at least one volatile agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin.

In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one environment-responsive agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one environment-responsive agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one environment-responsive agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film. In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one volatile agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film. In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one environment-responsive agent; at least one encapsulating agent; at least one bifunctional organopolysiloxane polymer, wherein the catalyst facilitates in situ cross-linking of the at least hydrosilylation step-growth polymerization reaction of the bifunctional organopolysiloxane polymer such that a film is formed on skin, thereby repairing the therapeutic film. In some embodiments, provided herein is a method for repairing a therapeutic film applied to a subject post-laser treatment including the steps of a) identifying an area of the film in need of repair; b) optionally smoothing the edges of the film; and c) applying a formulation for repairing the film, wherein a composition provided herein including a catalyst; at least one volatile agent; at least one encapsulating agent; at least one bifunctional organopolysiloxane polymer, wherein the catalyst facilitates in situ cross-linking of the at least hydrosilylation step-growth polymerization reaction of the bifunctional organopolysiloxane polymer such that a film is formed on skin, thereby repairing the therapeutic film.

In some embodiments, provided herein is a kit for repairing a therapeutic film used for post-laser treatment management, the kit including a composition provided herein including a catalyst; at least one environment-responsive agent; at least one ligand; at least one unsaturated organopolymer; and at least one hydride functionalized polysiloxane, such that a film is formed on skin. In some embodiments, provided herein is a kit for repairing a therapeutic film used for post-laser treatment management, the kit including a composition provided herein including a catalyst; at least one environment-responsive agent; at least one encapsulating agent; at least one unsaturated organopolymer; and at least one hydride functionalized polysiloxane, such that a film is formed on skin. In some embodiments, provided herein is a kit for repairing a therapeutic film used for post-laser treatment management, the kit including a composition provided herein including a catalyst; at least one environment-responsive agent; at least one ligand; at least one bifunctional organopolysiloxane polymer, such that a film is formed on skin. In some embodiments, provided herein is a kit for repairing a therapeutic film used for post-laser treatment management, the kit including a composition provided herein including a catalyst; at least one environment-responsive agent; at least one encapsulating agent; at least one bifunctional organopolysiloxane polymer, such that a film is formed on skin.

In some embodiments, provided herein is a kit for repairing a therapeutic film used for post-laser treatment management, the kit including a composition provided herein including a catalyst; at least one volatile agent; at least one ligand; at least one vinyl functionalized organopolysiloxane; and at least one hydride functionalized polysiloxane, such that a film is formed on skin. In some embodiments, provided herein is a kit for repairing a therapeutic film used for post-laser treatment management, the kit including a composition provided herein including a catalyst; at least one volatile agent; at least one encapsulating agent; at least one vinyl functionalized organopolysiloxane; and at least one hydride functionalized polysiloxane, such that a film is formed on skin. In some embodiments, provided herein is a kit for repairing a therapeutic film used for post-laser treatment management, the kit including a composition provided herein including a catalyst; at least one volatile agent; at least one ligand; at least one bifunctional organopolysiloxane polymer, such that a film is formed on skin. In some embodiments, provided herein is a kit for repairing a therapeutic film used for post-laser treatment management, the kit including a composition provided herein including a catalyst; at least one volatile agent; at least one encapsulating agent; at least one bifunctional organopolysiloxane polymer, such that a film is formed on skin.

In some embodiments, provided herein are methods for treating a subject post-light treatment, including applying to the subject a formulation including a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin, thereby treating a subject post-light treatment. In some embodiments, provided herein are methods for treating a subject post-light treatment, including applying to the subject a formulation including a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin, thereby treating a subject post-light treatment. In some embodiments, provided herein are methods for treating a subject post-light treatment, including applying to the subject a formulation including a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject post-light treatment. In some embodiments, provided herein are methods for treating a subject post-light treatment, including applying to the subject a formulation including a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject post-light treatment.

In some embodiments, provided herein are methods for treating a subject post-light treatment, including applying to the subject a formulation including a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin, thereby treating a subject post-light treatment. In some embodiments, provided herein are methods for treating a subject post-light treatment, including applying to the subject a formulation including a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin, thereby treating a subject post-light treatment. In some embodiments, provided herein are methods for treating a subject post-light treatment, including applying to the subject a formulation including a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject post-light treatment. In some embodiments, provided herein are methods for treating a subject post-light treatment, including applying to the subject a formulation including a composition provided herein including a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject post-light treatment.

In some embodiments, provided herein are non-invasive formulations that form a film upon application to a subject post light treatment, thereby facilitating healing of the subject post-light treatment. The invention also provides methods of using such formulations. In another embodiment, the invention provides cleansers to remove the film.

In some embodiments, provided herein is a composition for treating a subject post-light treatment, wherein the composition provided herein includes a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane upon application to skin, such that a film is formed on skin. In some embodiments, provided herein is a composition for treating a subject post-light treatment, wherein the composition provided herein includes a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane upon application to skin, such that a film is formed on skin. In some embodiments, provided herein is a composition for treating a subject post-light treatment, wherein the composition provided herein includes a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer upon application to skin, such that a film is formed on skin. In some embodiments, provided herein is a composition for treating a subject post-light treatment, wherein the composition provided herein includes a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer upon application to skin, such that a film is formed on skin.

In some embodiments, provided herein is a composition for treating a subject post-light treatment, wherein the composition provided herein includes a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane upon application to skin, such that a film is formed on skin. In some embodiments, provided herein is a composition for treating a subject post-light treatment, wherein the composition provided herein includes a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane upon application to skin, such that a film is formed on skin. In some embodiments, provided herein is a composition for treating a subject post-light treatment, wherein the composition provided herein includes a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer upon application to skin, such that a film is formed on skin. In some embodiments, provided herein is a composition for treating a subject post-light treatment, wherein the composition provided herein includes a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer upon application to skin, such that a film is formed on skin.

In some embodiments, provided herein are formulations for application to a subject post-light treatment that include a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin and the film has an appearance of natural skin. In some embodiments, provided herein are formulations for application to a subject post-light treatment that include a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin and the film has an appearance of natural skin. In some embodiments, provided herein are formulations for application to a subject post-light treatment that include a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin. In some embodiments, provided herein are formulations for application to a subject post-light treatment that include a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin.

In some embodiments, provided herein are formulations for application to a subject post-light treatment that include a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin and the film has an appearance of natural skin. In some embodiments, provided herein are formulations for application to a subject post-light treatment that include a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin and the film has an appearance of natural skin. In some embodiments, provided herein are formulations for application to a subject post-light treatment that include a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin. In some embodiments, provided herein are formulations for application to a subject post-light treatment that include a composition provided herein including a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin and the film has an appearance of natural skin.

In some embodiments, provided herein are films for treating a subject post-light treatment prepared by a process including the steps of: a) applying a composition provided herein including a catalyst; at least one environment-responsive agent; at least one ligand; at least one unsaturated organopolymer; and at least one hydride functionalized polysiloxane, in which the catalyst facilitates in situ cross-linking of the at least one unsaturated organopolymer; and at least one hydride functionalized polysiloxane, such that a film is formed on skin. In some embodiments, provided herein are films for treating a subject post-light treatment prepared by a process including the steps of: a) applying a composition provided herein including a catalyst; at least one environment-responsive agent; at least one encapsulating agent; at least one unsaturated organopolymer; and at least one hydride functionalized polysiloxane, in which the catalyst facilitates in situ cross-linking of the at least one unsaturated organopolymer; and at least one hydride functionalized polysiloxane, such that a film is formed on skin. In some embodiments, provided herein are films for treating a subject post-light treatment prepared by a process including the steps of: a) applying a composition provided herein including a catalyst; at least one environment-responsive agent; at least one ligand; at least one bifunctional organopolysiloxane polymer, in which the catalyst facilitates in situ hydrosilylation step-growth polymerization reaction of the bifunctional organopolysiloxane polymer, such that a film is formed on skin. In some embodiments, provided herein are films for treating a subject post-light treatment prepared by a process including the steps of: a) applying a composition provided herein including a catalyst; at least one environment-responsive agent; at least one encapsulating agent; at least one bifunctional organopolysiloxane polymer, in which the catalyst facilitates in situ hydrosilylation step-growth polymerization reaction of the bifunctional organopolysiloxane polymer, such that a film is formed on skin.

In some embodiments, provided herein are films for treating a subject post-light treatment prepared by a process including the steps of: a) applying a composition provided herein including a catalyst; at least one volatile agent; at least one ligand; at least one vinyl functionalized organopolysiloxane; and at least one hydride functionalized polysiloxane, in which the catalyst facilitates in situ cross-linking of the at least one vinyl functionalized organopolysiloxane; and at least one hydride functionalized polysiloxane, such that a film is formed on skin. In some embodiments, provided herein are films for treating a subject post-light treatment prepared by a process including the steps of: a) applying a composition provided herein including a catalyst; at least one volatile agent; at least one encapsulating agent; at least one vinyl functionalized organopolysiloxane; and at least one hydride functionalized polysiloxane, in which the catalyst facilitates in situ cross-linking of the at least one vinyl functionalized organopolysiloxane; and at least one hydride functionalized polysiloxane, such that a film is formed on skin. In some embodiments, provided herein are films for treating a subject post-light treatment prepared by a process including the steps of: a) applying a composition provided herein including a catalyst; at least one volatile agent; at least one ligand; at least one bifunctional organopolysiloxane polymer, in which the catalyst facilitates in situ hydrosilylation step-growth polymerization reaction of the bifunctional organopolysiloxane polymer, such that a film is formed on skin. In some embodiments, provided herein are films for treating a subject post-light treatment prepared by a process including the steps of: a) applying a composition provided herein including a catalyst; at least one volatile agent; at least one encapsulating agent; at least one bifunctional organopolysiloxane polymer, in which the catalyst facilitates in situ hydrosilylation step-growth polymerization reaction of the bifunctional organopolysiloxane polymer, such that a film is formed on skin.

In some embodiments, provided herein are methods for delivering an agent to a subject post-light treatment, including applying to the subject a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, thereby delivering the agent to the subject. In some embodiments, provided herein are methods for delivering an agent to a subject post-light treatment, including applying to the subject a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane such that a film is formed on skin, thereby delivering the agent to the subject. In some embodiments, provided herein are methods for delivering an agent to a subject post-light treatment, including applying to the subject a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer such that a film is formed on skin, thereby delivering the agent to the subject. In some embodiments, provided herein are methods for delivering an agent to a subject post-light treatment, including applying to the subject a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer such that a film is formed on skin, thereby delivering the agent to the subject.

In some embodiments, provided herein are methods for delivering an agent to a subject post-light treatment, including applying to the subject a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane such that a film is formed on skin, thereby delivering the agent to the subject. In some embodiments, provided herein are methods for delivering an agent to a subject post-light treatment, including applying to the subject a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane such that a film is formed on skin, thereby delivering the agent to the subject. In some embodiments, provided herein are methods for delivering an agent to a subject post-light treatment, including applying to the subject a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer such that a film is formed on skin, thereby delivering the agent to the subject. In some embodiments, provided herein are methods for delivering an agent to a subject post-light treatment, including applying to the subject a composition provided herein including a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer such that a film is formed on skin, thereby delivering the agent to the subject.

In some embodiments, provided herein is a kit for use in treating a post-light treatment on a subject in need thereof with a including a composition provided herein including a catalyst; at least one environment-responsive agent; at least one ligand; at least one unsaturated organopolymer; and at least one hydride functionalized polysiloxane; and instructions for use. In some embodiments, provided herein is a kit for use in treating a post-light treatment on a subject in need thereof with a including a composition provided herein including a catalyst; at least one environment-responsive agent; at least one encapsulating agent; at least one unsaturated organopolymer; and at least one hydride functionalized polysiloxane; and instructions for use. In some embodiments, provided herein is a kit for use in treating a post-light treatment on a subject in need thereof with a including a composition provided herein including a catalyst; at least one environment-responsive agent; at least one ligand; at least one bifunctional organopolysiloxane polymer; and instructions for use. In some embodiments, provided herein is a kit for use in treating a post-light treatment on a subject in need thereof with a including a composition provided herein including a catalyst; at least one environment-responsive agent; at least one encapsulating agent; at least one bifunctional organopolysiloxane polymer; and instructions for use.

In some embodiments, provided herein is a kit for use in treating a post-light treatment on a subject in need thereof with a including a composition provided herein including a catalyst; at least one volatile agent; at least one ligand; at least one vinyl functionalized organopolysiloxane; and at least one hydride functionalized polysiloxane; and instructions for use. In some embodiments, provided herein is a kit for use in treating a post-light treatment on a subject in need thereof with a including a composition provided herein including a catalyst; at least one volatile agent; at least one encapsulating agent; at least one vinyl functionalized organopolysiloxane; and at least one hydride functionalized polysiloxane; and instructions for use. In some embodiments, provided herein is a kit for use in treating a post-light treatment on a subject in need thereof with a including a composition provided herein including a catalyst; at least one volatile agent; at least one ligand; at least one bifunctional organopolysiloxane polymer; and instructions for use. In some embodiments, provided herein is a kit for use in treating a post-light treatment on a subject in need thereof with a including a composition provided herein including a catalyst; at least one volatile agent; at least one encapsulating agent; at least one bifunctional organopolysiloxane polymer; and instructions for use.

In some embodiments, provided herein are therapeutic formulations for application to a subject post-light treatment, including at least one preselected function modulating component, in which the composition forms a therapeutic film upon application to the subject.

In some embodiments, provided herein are therapeutic formulations for application to a subject post-light treatment on the subject that target a treatment area on a subject, wherein the targeted area includes an area that has been at least partially light-treated, including at least one preselected treatment specific component, wherein the composition forms a therapeutic film upon application to the target treatment area on the subject.

In some embodiments, provided herein is a film removing cleanser for use in removing a therapeutic film used for post-light treatment recovery management, wherein the film is prepared by a process including the steps of applying a composition provided herein including a catalyst; at least one environment-responsive agent; at least one ligand; at least one unsaturated organopolymer; and at least one hydride functionalized polysiloxane, and wherein said catalyst facilitates in situ cross-linking of the at least one unsaturated organopolymer; and at least one hydride functionalized polysiloxane. In some embodiments, provided herein is a film removing cleanser for use in removing a therapeutic film used for post-light treatment recovery management, wherein the film is prepared by a process including the steps of applying a composition provided herein including a catalyst; at least one environment-responsive agent; at least one encapsulating agent; at least one unsaturated organopolymer; and at least one hydride functionalized polysiloxane, and wherein said catalyst facilitates in situ cross-linking of the at least one unsaturated organopolymer; and at least one hydride functionalized polysiloxane. In some embodiments, provided herein is a film removing cleanser for use in removing a therapeutic film used for post-light treatment recovery management, wherein the film is prepared by a process including the steps of applying a composition provided herein including a catalyst; at least one environment-responsive agent; at least one ligand; at least one bifunctional organopolysiloxane polymer, and wherein said catalyst facilitates in situ hydrosilylation step-growth polymerization reaction of the bifunctional organopolysiloxane polymer. In some embodiments, provided herein is a film removing cleanser for use in removing a therapeutic film used for post-light treatment recovery management, wherein the film is prepared by a process including the steps of applying a composition provided herein including a catalyst; at least one environment-responsive agent; at least one encapsulating agent; at least one bifunctional organopolysiloxane polymer, and wherein said catalyst facilitates in situ hydrosilylation step-growth polymerization reaction of the bifunctional organopolysiloxane polymer.

In some embodiments, provided herein is a film removing cleanser for use in removing a therapeutic film used for post-light treatment recovery management, wherein the film is prepared by a process including the steps of applying a composition provided herein including a catalyst; at least one volatile agent; at least one ligand; at least one vinyl functionalized organopolysiloxane; and at least one hydride functionalized polysiloxane, and wherein said catalyst facilitates in situ cross-linking of the at least one vinyl functionalized organopolysiloxane; and at least one hydride functionalized polysiloxane. In some embodiments, provided herein is a film removing cleanser for use in removing a therapeutic film used for post-light treatment recovery management, wherein the film is prepared by a process including the steps of applying a composition provided herein including a catalyst; at least one volatile agent; at least one encapsulating agent; at least one vinyl functionalized organopolysiloxane; and at least one hydride functionalized polysiloxane, and wherein said catalyst facilitates in situ cross-linking of the at least one vinyl functionalized organopolysiloxane; and at least one hydride functionalized polysiloxane. In some embodiments, provided herein is a film removing cleanser for use in removing a therapeutic film used for post-light treatment recovery management, wherein the film is prepared by a process including the steps of applying a composition provided herein including a catalyst; at least one volatile agent; at least one ligand; at least one bifunctional organopolysiloxane polymer, and wherein said catalyst facilitates in situ hydrosilylation step-growth polymerization reaction of the bifunctional organopolysiloxane polymer. In some embodiments, provided herein is a film removing cleanser for use in removing a therapeutic film used for post-light treatment recovery management, wherein the film is prepared by a process including the steps of applying a composition provided herein including a catalyst; at least one volatile agent; at least one encapsulating agent; at least one bifunctional organopolysiloxane polymer, and wherein said catalyst facilitates in situ hydrosilylation step-growth polymerization reaction of the bifunctional organopolysiloxane polymer.

In some embodiments, provided herein is a film removing cleanser including a film wetting component, a penetration component, a film swelling component and a film release component.

In some embodiments, provided herein is a formulation for repairing a therapeutic film applied to a subject post-light treatment, wherein said formulation includes a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a formulation for repairing a therapeutic film applied to a subject post-light treatment, wherein said formulation includes a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a formulation for repairing a therapeutic film applied to a subject post-light treatment, wherein said formulation includes a composition provided herein including a catalyst; at least one environment-responsive agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a formulation for repairing a therapeutic film applied to a subject post-light treatment, wherein said formulation includes a composition provided herein including a catalyst; at least one environment-responsive agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin.

In some embodiments, provided herein is a formulation for repairing a therapeutic film applied to a subject post-light treatment, wherein said formulation includes a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a formulation for repairing a therapeutic film applied to a subject post-light treatment, wherein said formulation includes a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a formulation for repairing a therapeutic film applied to a subject post-light treatment, wherein said formulation includes a composition provided herein including a catalyst; at least one volatile agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a formulation for repairing a therapeutic film applied to a subject post-light treatment, wherein said formulation includes a composition provided herein including a catalyst; at least one volatile agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin.

In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin, thereby repairing the therapeutic film. In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin, thereby repairing the therapeutic film. In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one environment-responsive agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film. In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one environment-responsive agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film.

In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one volatile agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film. In some embodiments, provided herein is a method for repairing a therapeutic film applied to a subject post-light treatment including the steps of a) identifying an area of the film in need of repair; b) optionally smoothing the edges of the film; and c) applying a formulation for repairing the film, wherein the formulation provided herein includes a catalyst; at least one volatile agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film.

In some embodiments, provided herein is a kit for repairing a therapeutic film used for post-light treatment management, the kit including a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a kit for repairing a therapeutic film used for post-light treatment management, the kit including a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a kit for repairing a therapeutic film used for post-light treatment management, the kit including a composition provided herein including a catalyst; at least one environment-responsive agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a kit for repairing a therapeutic film used for post-light treatment management, the kit including a composition provided herein including a catalyst; at least one environment-responsive agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin.

In some embodiments, provided herein is a kit for repairing a therapeutic film used for post-light treatment management, the kit including a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a kit for repairing a therapeutic film used for post-light treatment management, the kit including a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a kit for repairing a therapeutic film used for post-light treatment management, the kit including a composition provided herein including a catalyst; at least one volatile agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a kit for repairing a therapeutic film used for post-light treatment management, the kit including a composition provided herein including a catalyst; at least one volatile agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin.

In some embodiments, provided herein are methods for treating a subject after a chemical peel treatment, including applying to the subject a composition provided herein including a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin, thereby treating a subject after a chemical peel treatment. In some embodiments, provided herein are methods for treating a subject after a chemical peel treatment, including applying to the subject a composition provided herein including a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane, such that a film is formed on skin, thereby treating a subject after a chemical peel treatment. In some embodiments, provided herein are methods for treating a subject after a chemical peel treatment, including applying to the subject a composition provided herein including a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject after a chemical peel treatment. In some embodiments, provided herein are methods for treating a subject after a chemical peel treatment, including applying to the subject a composition provided herein including a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject after a chemical peel treatment.

In some embodiments, provided herein are methods for treating a subject after a chemical peel treatment, including applying to the subject a composition provided herein including a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin, thereby treating a subject after a chemical peel treatment. In some embodiments, provided herein are methods for treating a subject after a chemical peel treatment, including applying to the subject a composition provided herein including a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane, such that a film is formed on skin, thereby treating a subject after a chemical peel treatment. In some embodiments, provided herein are methods for treating a subject after a chemical peel treatment, including applying to the subject a composition provided herein including a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject after a chemical peel treatment. In some embodiments, provided herein are methods for treating a subject after a chemical peel treatment, including applying to the subject a composition provided herein including a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer, such that a film is formed on skin, thereby treating a subject after a chemical peel treatment.

In some embodiments, provided herein are non-invasive formulations that form a film upon application to a subject post laser treatment, thereby facilitating healing of the subject after a chemical peel treatment. The invention also provides methods of using such formulations. In another embodiment, the invention provides cleansers to remove the film.

In some embodiments, provided herein is a composition for treating a subject after a chemical peel treatment, wherein the composition provided herein includes a catalyst; at least one environment-responsive agent; at least one ligand; at least one unsaturated organopolymer; and at least one hydride functionalized polysiloxane; in which the catalyst facilitates in situ cross-linking of the at least one unsaturated organopolymer; and at least one hydride functionalized polysiloxane upon application to skin, such that a film is formed on skin. In some embodiments, provided herein is a composition for treating a subject after a chemical peel treatment, wherein the composition provided herein includes a catalyst; at least one environment-responsive agent; at least one encapsulating agent; at least one unsaturated organopolymer; and at least one hydride functionalized polysiloxane; in which the catalyst facilitates in situ cross-linking of the at least one unsaturated organopolymer; and at least one hydride functionalized polysiloxane upon application to skin, such that a film is formed on skin. In some embodiments, provided herein is a composition for treating a subject after a chemical peel treatment, wherein the composition provided herein includes a catalyst; at least one environment-responsive agent; at least one ligand; at least one bifunctional organopolysiloxane polymer; in which the catalyst facilitates in situ hydrosilylation step-growth polymerization reaction of the bifunctional organopolysiloxane polymer upon application to skin, such that a film is formed on skin. In some embodiments, provided herein is a composition for treating a subject after a chemical peel treatment, wherein the composition provided herein includes a catalyst; at least one environment-responsive agent; at least one encapsulating agent; at least one bifunctional organopolysiloxane polymer; in which the catalyst facilitates in situ hydrosilylation step-growth polymerization reaction of the bifunctional organopolysiloxane polymer upon application to skin, such that a film is formed on skin.

In some embodiments, provided herein is a composition for treating a subject after a chemical peel treatment, wherein the composition provided herein includes a catalyst; at least one volatile agent; at least one ligand; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane upon application to skin, such that a film is formed on skin. In some embodiments, provided herein is a composition for treating a subject after a chemical peel treatment, wherein the composition provided herein includes a catalyst; at least one volatile agent; at least one encapsulating agent; at least one vinyl functionalized organopolysiloxane; and at least one hydride functionalized polysiloxane; in which the catalyst facilitates in situ cross-linking of the at least one vinyl functionalized organopolysiloxane; and at least one hydride functionalized polysiloxane upon application to skin, such that a film is formed on skin. In some embodiments, provided herein is a composition for treating a subject after a chemical peel treatment, wherein the composition provided herein includes a catalyst; at least one volatile agent; at least one ligand; at least one bifunctional organopolysiloxane polymer; in which the catalyst facilitates in situ hydrosilylation step-growth polymerization reaction of the bifunctional organopolysiloxane polymer upon application to skin, such that a film is formed on skin. In some embodiments, provided herein is a composition for treating a subject after a chemical peel treatment, wherein the composition provided herein includes a catalyst; at least one volatile agent; at least one encapsulating agent; at least one bifunctional organopolysiloxane polymer; in which the catalyst facilitates in situ hydrosilylation step-growth polymerization reaction of the bifunctional organopolysiloxane polymer upon application to skin, such that a film is formed on skin.

In some embodiments, provided herein are films for treating a subject after a chemical peel treatment prepared by a process including the steps of: applying a composition provided herein including a catalyst; at least one environment-responsive agent; at least one ligand; at least one unsaturated organopolymer; and at least one hydride functionalized polysiloxane, in which the catalyst facilitates in situ cross-linking of the at least one unsaturated organopolymer; and at least one hydride functionalized polysiloxane, such that a film is formed on skin. In some embodiments, provided herein are films for treating a subject after a chemical peel treatment prepared by a process including the steps of: applying a composition provided herein including a catalyst; at least one environment-responsive agent; at least one encapsulating agent; at least one unsaturated organopolymer; and at least one hydride functionalized polysiloxane, in which the catalyst facilitates in situ cross-linking of the at least one unsaturated organopolymer; and at least one hydride functionalized polysiloxane, such that a film is formed on skin. In some embodiments, provided herein are films for treating a subject after a chemical peel treatment prepared by a process including the steps of: applying a composition provided herein including a catalyst; at least one environment-responsive agent; at least one ligand; at least one bifunctional organopolysiloxane polymer, in which the catalyst facilitates in situ hydrosilylation step-growth polymerization reaction of the bifunctional organopolysiloxane polymer, such that a film is formed on skin. In some embodiments, provided herein are films for treating a subject after a chemical peel treatment prepared by a process including the steps of: applying a composition provided herein including a catalyst; at least one environment-responsive agent; at least one encapsulating agent; at least one bifunctional organopolysiloxane polymer, in which the catalyst facilitates in situ hydrosilylation step-growth polymerization reaction of the bifunctional organopolysiloxane polymer, such that a film is formed on skin.

In some embodiments, provided herein are films for treating a subject after a chemical peel treatment prepared by a process including the steps of: applying a composition provided herein including a catalyst; at least one volatile agent; at least one ligand; at least one vinyl functionalized organopolysiloxane; and at least one hydride functionalized polysiloxane, in which the catalyst facilitates in situ cross-linking of the at least one vinyl functionalized organopolysiloxane; and at least one hydride functionalized polysiloxane, such that a film is formed on skin. In some embodiments, provided herein are films for treating a subject after a chemical peel treatment prepared by a process including the steps of: applying a composition provided herein including a catalyst; at least one volatile agent; at least one encapsulating agent; at least one vinyl functionalized organopolysiloxane; and at least one hydride functionalized polysiloxane, in which the catalyst facilitates in situ cross-linking of the at least one vinyl functionalized organopolysiloxane; and at least one hydride functionalized polysiloxane, such that a film is formed on skin. In some embodiments, provided herein are films for treating a subject after a chemical peel treatment prepared by a process including the steps of: applying a composition provided herein including a catalyst; at least one volatile agent; at least one ligand; at least one bifunctional organopolysiloxane polymer, in which the catalyst facilitates in situ hydrosilylation step-growth polymerization reaction of the bifunctional organopolysiloxane polymer, such that a film is formed on skin. In some embodiments, provided herein are films for treating a subject after a chemical peel treatment prepared by a process including the steps of: applying a composition provided herein including a catalyst; at least one volatile agent; at least one encapsulating agent; at least one bifunctional organopolysiloxane polymer, in which the catalyst facilitates in situ hydrosilylation step-growth polymerization reaction of the bifunctional organopolysiloxane polymer, such that a film is formed on skin.

In some embodiments, provided herein are methods for delivering an agent to a subject after a chemical peel treatment, including applying to the subject a composition provided herein including a catalyst; at least one environment-responsive agent; at least one ligand; at least one unsaturated organopolymer; and at least one hydride functionalized polysiloxane, optionally further including one or more agents; and b) a catalyst optionally including one or more agents; in which the catalyst facilitates in situ cross-linking of the at least one unsaturated organopolymer; and at least one hydride functionalized polysiloxane such that a film is formed on skin, thereby delivering the agent to the subject. In some embodiments, provided herein are methods for delivering an agent to a subject after a chemical peel treatment, including applying to the subject a composition provided herein including a catalyst; at least one environment-responsive agent; at least one encapsulating agent; at least one unsaturated organopolymer; and at least one hydride functionalized polysiloxane, optionally further including one or more agents; and b) a catalyst optionally including one or more agents; in which the catalyst facilitates in situ cross-linking of the at least one unsaturated organopolymer; and at least one hydride functionalized polysiloxane such that a film is formed on skin, thereby delivering the agent to the subject. In some embodiments, provided herein are methods for delivering an agent to a subject after a chemical peel treatment, including applying to the subject a composition provided herein including a catalyst; at least one environment-responsive agent; at least one ligand; at least one bifunctional organopolysiloxane polymer, optionally further including one or more agents; and b) a catalyst optionally including one or more agents; in which the catalyst facilitates in situ hydrosilylation step-growth polymerization reaction of the bifunctional organopolysiloxane polymer such that a film is formed on skin, thereby delivering the agent to the subject. In some embodiments, provided herein are methods for delivering an agent to a subject after a chemical peel treatment, including applying to the subject a composition provided herein including a catalyst; at least one environment-responsive agent; at least one encapsulating agent; at least one bifunctional organopolysiloxane polymer, optionally further including one or more agents; and b) a catalyst optionally including one or more agents; in which the catalyst facilitates in situ hydrosilylation step-growth polymerization reaction of the bifunctional organopolysiloxane polymer such that a film is formed on skin, thereby delivering the agent to the subject.

In some embodiments, provided herein are methods for delivering an agent to a subject after a chemical peel treatment, including applying to the subject a composition provided herein including a catalyst; at least one volatile agent; at least one ligand; at least one vinyl functionalized organopolysiloxane; and at least one hydride functionalized polysiloxane, optionally further including one or more agents; and b) a catalyst optionally including one or more agents; in which the catalyst facilitates in situ cross-linking of the at least one vinyl functionalized organopolysiloxane; and at least one hydride functionalized polysiloxane such that a film is formed on skin, thereby delivering the agent to the subject. In some embodiments, provided herein are methods for delivering an agent to a subject after a chemical peel treatment, including applying to the subject a composition provided herein including a catalyst; at least one volatile agent; at least one encapsulating agent; at least one vinyl functionalized organopolysiloxane; and at least one hydride functionalized polysiloxane, optionally further including one or more agents; and b) a catalyst optionally including one or more agents; in which the catalyst facilitates in situ cross-linking of the at least one vinyl functionalized organopolysiloxane; and at least one hydride functionalized polysiloxane such that a film is formed on skin, thereby delivering the agent to the subject. In some embodiments, provided herein are methods for delivering an agent to a subject after a chemical peel treatment, including applying to the subject a composition provided herein including a catalyst; at least one volatile agent; at least one ligand; at least one bifunctional organopolysiloxane polymer, optionally further including one or more agents; and b) a catalyst optionally including one or more agents; in which the catalyst facilitates in situ hydrosilylation step-growth polymerization reaction of the bifunctional organopolysiloxane polymer such that a film is formed on skin, thereby delivering the agent to the subject. In some embodiments, provided herein are methods for delivering an agent to a subject after a chemical peel treatment, including applying to the subject a composition provided herein including a catalyst; at least one volatile agent; at least one encapsulating agent; at least one bifunctional organopolysiloxane polymer, optionally further including one or more agents; and b) a catalyst optionally including one or more agents; in which the catalyst facilitates in situ hydrosilylation step-growth polymerization reaction of the bifunctional organopolysiloxane polymer such that a film is formed on skin, thereby delivering the agent to the subject.

In some embodiments, provided herein is a kit for use in treating a after a chemical peel treatment on a subject in need thereof with a composition provided herein including a catalyst; at least one environment-responsive agent; at least one ligand; at least one unsaturated organopolymer; and at least one hydride functionalized polysiloxane; and instructions for use. In some embodiments, provided herein is a kit for use in treating a after a chemical peel treatment on a subject in need thereof with a composition provided herein including a catalyst; at least one environment-responsive agent; at least one encapsulating agent; at least one unsaturated organopolymer; and at least one hydride functionalized polysiloxane; and instructions for use. In some embodiments, provided herein is a kit for use in treating a after a chemical peel treatment on a subject in need thereof with a composition provided herein including a catalyst; at least one environment-responsive agent; at least one ligand; at least one bifunctional organopolysiloxane polymer; and instructions for use. In some embodiments, provided herein is a kit for use in treating a after a chemical peel treatment on a subject in need thereof with a composition provided herein including a catalyst; at least one environment-responsive agent; at least one encapsulating agent; at least one bifunctional organopolysiloxane polymer; and instructions for use.

In some embodiments, provided herein is a kit for use in treating a after a chemical peel treatment on a subject in need thereof with a composition provided herein including a catalyst; at least one volatile agent; at least one ligand; at least one vinyl functionalized organopolysiloxane; and at least one hydride functionalized polysiloxane; and instructions for use. In some embodiments, provided herein is a kit for use in treating a after a chemical peel treatment on a subject in need thereof with a composition provided herein including a catalyst; at least one volatile agent; at least one encapsulating agent; at least one vinyl functionalized organopolysiloxane; and at least one hydride functionalized polysiloxane; and instructions for use. In some embodiments, provided herein is a kit for use in treating a after a chemical peel treatment on a subject in need thereof with a composition provided herein including a catalyst; at least one volatile agent; at least one ligand; at least one bifunctional organopolysiloxane polymer; and instructions for use. In some embodiments, provided herein is a kit for use in treating a after a chemical peel treatment on a subject in need thereof with a composition provided herein including a catalyst; at least one volatile agent; at least one encapsulating agent; at least one bifunctional organopolysiloxane polymer; and instructions for use.

In some embodiments, provided herein are therapeutic formulations for application to a subject after a chemical peel treatment, including at least one preselected function modulating component, in which the composition forms a therapeutic film upon application to the subject.

In some embodiments, provided herein are therapeutic formulations for application to a subject after a chemical peel treatment on the subject that target a treatment area on a subject, wherein the targeted area includes an area that has been at least partially laser-treated, including at least one preselected treatment specific component, wherein the composition forms a therapeutic film upon application to the target treatment area on the subject.

In some embodiments, provided herein is a film removing cleanser for use in removing a therapeutic film used after a chemical peel treatment, wherein the film is prepared by a process including the steps of applying a composition provided herein including a catalyst; at least one environment-responsive agent; at least one ligand; at least one unsaturated organopolymer; and at least one hydride functionalized polysiloxane, wherein said catalyst facilitates in situ cross-linking of the at least one unsaturated organopolymer; and at least one hydride functionalized polysiloxane. In some embodiments, provided herein is a film removing cleanser for use in removing a therapeutic film used after a chemical peel treatment, wherein the film is prepared by a process including the steps of applying a composition provided herein including a catalyst; at least one environment-responsive agent; at least one encapsulating agent; at least one unsaturated organopolymer; and at least one hydride functionalized polysiloxane, wherein said catalyst facilitates in situ cross-linking of the at least one unsaturated organopolymer; and at least one hydride functionalized polysiloxane. In some embodiments, provided herein is a film removing cleanser for use in removing a therapeutic film used after a chemical peel treatment, wherein the film is prepared by a process including the steps of applying a composition provided herein including a catalyst; at least one environment-responsive agent; at least one ligand; at least one bifunctional organopolysiloxane polymer, wherein said catalyst facilitates in situ hydrosilylation step-growth polymerization reaction of the bifunctional organopolysiloxane polymer. In some embodiments, provided herein is a film removing cleanser for use in removing a therapeutic film used after a chemical peel treatment, wherein the film is prepared by a process including the steps of applying a composition provided herein including a catalyst; at least one environment-responsive agent; at least one encapsulating agent; at least bifunctional organopolysiloxane polymer, wherein said catalyst facilitates in situ hydrosilylation step-growth polymerization reaction of the bifunctional organopolysiloxane polymer.

In some embodiments, provided herein is a film removing cleanser for use in removing a therapeutic film used after a chemical peel treatment, wherein the film is prepared by a process including the steps of applying a composition provided herein including a catalyst; at least one volatile agent; at least one ligand; at least one vinyl functionalized organopolysiloxane; and at least one hydride functionalized polysiloxane, wherein said catalyst facilitates in situ cross-linking of the at least one vinyl functionalized organopolysiloxane; and at least one hydride functionalized polysiloxane. In some embodiments, provided herein is a film removing cleanser for use in removing a therapeutic film used after a chemical peel treatment, wherein the film is prepared by a process including the steps of applying a composition provided herein including a catalyst; at least one volatile agent; at least one encapsulating agent; at least one vinyl functionalized organopolysiloxane; and at least one hydride functionalized polysiloxane, wherein said catalyst facilitates in situ cross-linking of the at least one vinyl functionalized organopolysiloxane; and at least one hydride functionalized polysiloxane. In some embodiments, provided herein is a film removing cleanser for use in removing a therapeutic film used after a chemical peel treatment, wherein the film is prepared by a process including the steps of applying a composition provided herein including a catalyst; at least one volatile agent; at least one ligand; at least one bifunctional organopolysiloxane polymer, wherein said catalyst facilitates in situ hydrosilylation step-growth polymerization reaction of the bifunctional organopolysiloxane polymer. In some embodiments, provided herein is a film removing cleanser for use in removing a therapeutic film used after a chemical peel treatment, wherein the film is prepared by a process including the steps of applying a composition provided herein including a catalyst; at least one volatile agent; at least one encapsulating agent; at least bifunctional organopolysiloxane polymer, wherein said catalyst facilitates in situ hydrosilylation step-growth polymerization reaction of the bifunctional organopolysiloxane polymer.

In some embodiments, provided herein is a film removing cleanser including a film wetting component, a penetration component, a film swelling component and a film release component.

In some embodiments, provided herein is a formulation for repairing a therapeutic film applied to a subject after a chemical peel treatment, wherein said formulation provided herein includes a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a formulation for repairing a therapeutic film applied to a subject after a chemical peel treatment, wherein said formulation provided herein includes a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin. In some embodiments, provided herein is a formulation for repairing a therapeutic film applied to a subject after a chemical peel treatment, wherein said formulation provided herein includes a catalyst; at least one environment-responsive agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a formulation for repairing a therapeutic film applied to a subject after a chemical peel treatment, wherein said formulation provided herein includes a catalyst; at least one environment-responsive agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin.

In some embodiments, provided herein is a formulation for repairing a therapeutic film applied to a subject after a chemical peel treatment, wherein said formulation provided herein includes a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a formulation for repairing a therapeutic film applied to a subject after a chemical peel treatment, wherein said formulation provided herein includes a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin. In some embodiments, provided herein is a formulation for repairing a therapeutic film applied to a subject after a chemical peel treatment, wherein said formulation provided herein includes a catalyst; at least one volatile agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin. In some embodiments, provided herein is a formulation for repairing a therapeutic film applied to a subject after a chemical peel treatment, wherein said formulation provided herein includes a catalyst; at least one volatile agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin.

In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin, thereby repairing the therapeutic film. In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer; and at least one hydride functionalized polysiloxane such that a film is formed on skin, thereby repairing the therapeutic film. In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one environment-responsive agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film. In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one environment-responsive agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film.

In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane; and at least one hydride functionalized polysiloxane such that a film is formed on skin, thereby repairing the therapeutic film. In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one volatile agent; at least one ligand; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film. In some embodiments, provided herein is a method for repairing a therapeutic film applied to a subject after a chemical peel treatment including the steps of a) identifying an area of the film in need of repair; b) optionally smoothing the edges of the film; and c) applying a formulation for repairing the film, wherein the formulation includes a catalyst; at least one volatile agent; at least one encapsulating agent; at least one bifunctional organopolysiloxane polymer, wherein the catalyst facilitates in situ hydrosilylation step-growth polymerization reaction of the bifunctional organopolysiloxane polymer such that a film is formed on skin, thereby repairing the therapeutic film.

### 1.17 Kits For Use With The Compositions And Methods Provided Herein

In some aspects, provided herein is a kit for use in treating a post-laser treatment on a subject in need thereof with a composition provided herein including a catalyst; at least one environment-responsive agent; at least one ligand; at least one unsaturated organopolymer; and at least one hydride functionalized polysiloxane and c) instructions for use. In some aspects, provided herein is a kit for use in treating a post-laser treatment on a subject in need thereof with a composition provided herein including a catalyst; at least one environment-responsive agent; at least one encapsulating agent; at least one unsaturated organopolymer; and at least one hydride functionalized polysiloxane and c) instructions for use. In some aspects, provided herein is a kit for use in treating a post-laser treatment on a subject in need thereof with a composition provided herein including a catalyst; at least one environment-responsive agent; at least one ligand; at least one bifunctional organopolysiloxane polymer and c) instructions for use. In some aspects, provided herein is a kit for use in treating a post-laser treatment on a subject in need thereof with a composition provided herein including a catalyst; at least one environment-responsive agent; at least one encapsulating agent; at least bifunctional organopolysiloxane polymer and c) instructions for use.

In some aspects, provided herein is a kit for use in treating a post-laser treatment on a subject in need thereof with a composition provided herein including a catalyst; at least one volatile agent; at least one ligand; at least one vinyl functionalized organopolysiloxane; and at least one hydride functionalized polysiloxane and c) instructions for use. In some aspects, provided herein is a kit for use in treating a post-laser treatment on a subject in need thereof with a composition provided herein including a catalyst; at least one volatile agent; at least one encapsulating agent; at least one vinyl functionalized organopolysiloxane; and at least one hydride functionalized polysiloxane and c) instructions for use. In some aspects, provided herein is a kit for use in treating a post-laser treatment on a subject in need thereof with a composition provided herein including a catalyst; at least one volatile agent; at least one ligand; at least one bifunctional organopolysiloxane polymer and c) instructions for use. In some aspects, provided herein is a kit for use in treating a post-laser treatment on a subject in need thereof with a composition provided herein including a catalyst; at least one volatile agent; at least one encapsulating agent; at least bifunctional organopolysiloxane polymer and c) instructions for use.

In some embodiments, provided herein is a kit for repairing a therapeutic film used for post-laser treatment management, the kit including a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used for post-laser treatment management, the kit including a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used for post-laser treatment management, the kit including a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used for post-laser treatment management, the kit including a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin.

In some embodiments, provided herein is a kit for repairing a therapeutic film used for post-laser treatment management, the kit including a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used for post-laser treatment management, the kit including a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used for post-laser treatment management, the kit including a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used for post-laser treatment management, the kit including a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin.

In some aspects, provided herein is a kit for use in treating a post-light treatment on a subject in need thereof with a composition provided herein including a catalyst; at least one environment-responsive agent; at least one ligand; at least one unsaturated organopolymer; and at least one hydride functionalized polysiloxane and instructions for use. In some aspects, provided herein is a kit for use in treating a post-light treatment on a subject in need thereof with a composition provided herein including a catalyst; at least one environment-responsive agent; at least one encapsulating agent; at least one unsaturated organopolymer; and at least one hydride functionalized polysiloxane and instructions for use. In some aspects, provided herein is a kit for use in treating a post-light treatment on a subject in need thereof with a composition provided herein including a catalyst; at least one environment-responsive agent; at least one ligand; at least one unsaturated organopolymer; and at least one hydride functionalized polysiloxane and instructions for use. In some aspects, provided herein is a kit for use in treating a post-light treatment on a subject in need thereof with a composition provided herein including a catalyst; at least one environment-responsive agent; at least one encapsulating agent; at least one unsaturated organopolymer; and at least one hydride functionalized polysiloxane and instructions for use.

In some aspects, provided herein is a kit for use in treating a post-light treatment on a subject in need thereof with a composition provided herein including a catalyst; at least one volatile agent; at least one ligand; at least one vinyl functionalized organopolysiloxane; and at least one hydride functionalized polysiloxane and instructions for use. In some aspects, provided herein is a kit for use in treating a post-light treatment on a subject in need thereof with a composition provided herein including a catalyst; at least one volatile agent; at least one encapsulating agent; at least one vinyl functionalized organopolysiloxane; and at least one hydride functionalized polysiloxane and instructions for use. In some aspects, provided herein is a kit for use in treating a post-light treatment on a subject in need thereof with a composition provided herein including a catalyst; at least one volatile agent; at least one ligand; at least one vinyl functionalized organopolysiloxane; and at least one hydride functionalized polysiloxane and instructions for use. In some aspects, provided herein is a kit for use in treating a post-light treatment on a subject in need thereof with a composition provided herein including a catalyst; at least one volatile agent; at least one encapsulating agent; at least one vinyl functionalized organopolysiloxane; and at least one hydride functionalized polysiloxane and instructions for use.

In some embodiments, provided herein is a kit for repairing a therapeutic film used for post-light treatment management, the kit including a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used for post-light treatment management, the kit including a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used for post-light treatment management, the kit including a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used for post-light treatment management, the kit including a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin.

In some embodiments, provided herein is a kit for repairing a therapeutic film used for post-light treatment management, the kit including a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used for post-light treatment management, the kit including a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used for post-light treatment management, the kit including a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used for post-light treatment management, the kit including a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin.

In some aspects, provided herein is a kit for use in treating a after a chemical peel treatment on a subject in need thereof with a composition provided herein including a catalyst; at least one environment-responsive agent; at least one ligand; at least one unsaturated organopolymer; and at least one hydride functionalized polysiloxane and instructions for use. In some aspects, provided herein is a kit for use in treating a after a chemical peel treatment on a subject in need thereof with a composition provided herein including a catalyst; at least one environment-responsive agent; at least one encapsulating agent; at least one unsaturated organopolymer; and at least one hydride functionalized polysiloxane and instructions for use. In some aspects, provided herein is a kit for use in treating a after a chemical peel treatment on a subject in need thereof with a composition provided herein including a catalyst; at least one environment-responsive agent; at least one ligand; at least one unsaturated organopolymer; and at least one hydride functionalized polysiloxane and instructions for use. In some aspects, provided herein is a kit for use in treating a after a chemical peel treatment on a subject in need thereof with a composition provided herein including a catalyst; at least one environment-responsive agent; at least one encapsulating agent; at least one unsaturated organopolymer; and at least one hydride functionalized polysiloxane and instructions for use.

In some aspects, provided herein is a kit for use in treating a after a chemical peel treatment on a subject in need thereof with a composition provided herein including a catalyst; at least one volatile agent; at least one ligand; at least one vinyl functionalized organopolysiloxane; and at least one hydride functionalized polysiloxane and instructions for use. In some aspects, provided herein is a kit for use in treating a after a chemical peel treatment on a subject in need thereof with a composition provided herein including a catalyst; at least one volatile agent; at least one encapsulating agent; at least one vinyl functionalized organopolysiloxane; and at least one hydride functionalized polysiloxane and instructions for use. In some aspects, provided herein is a kit for use in treating a after a chemical peel treatment on a subject in need thereof with a composition provided herein including a catalyst; at least one volatile agent; at least one ligand; at least one vinyl functionalized organopolysiloxane; and at least one hydride functionalized polysiloxane and instructions for use. In some aspects, provided herein is a kit for use in treating a after a chemical peel treatment on a subject in need thereof with a composition provided herein including a catalyst; at least one volatile agent; at least one encapsulating agent; at least one vinyl functionalized organopolysiloxane; and at least one hydride functionalized polysiloxane and instructions for use.

In some embodiments, provided herein is a kit for repairing a therapeutic film used after a chemical peel treatment, the kit including a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used after a chemical peel treatment, the kit including a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used after a chemical peel treatment, the kit including a composition provided herein including a catalyst; at least one environment-responsive agent; at least one ligand; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin. In some embodiments, provided herein is a kit for repairing a therapeutic film used after a chemical peel treatment, the kit including a composition provided herein including a catalyst; at least one environment-responsive agent; at least one encapsulating agent; at least one unsaturated organopolymer; and at least one hydride functionalized polysiloxane, wherein the catalyst facilitates in situ cross-linking of the at least one unsaturated organopolymer and at least one hydride functionalized polysiloxane such that a film is formed on skin.

In some embodiments, provided herein is a kit for repairing a therapeutic film used after a chemical peel treatment, the kit including a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used after a chemical peel treatment, the kit including a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used after a chemical peel treatment, the kit including a composition provided herein including a catalyst; at least one volatile agent; at least one ligand; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin. In some embodiments, provided herein is a kit for repairing a therapeutic film used after a chemical peel treatment, the kit including a composition provided herein including a catalyst; at least one volatile agent; at least one encapsulating agent; at least one vinyl functionalized organopolysiloxane; and at least one hydride functionalized polysiloxane, wherein the catalyst facilitates in situ cross-linking of the at least one vinyl functionalized organopolysiloxane and at least one hydride functionalized polysiloxane such that a film is formed on skin.

In some embodiments, provided herein is a kit including a therapeutic formulation including a composition provided herein including a catalyst; at least one environment-responsive agent; at least one ligand; at least one unsaturated organopolymer; and at least one hydride functionalized polysiloxane. In some embodiments, the kit further includes instructions for use of the kit, one or more brushes, one or more swabs, a film removing cleanser or a mirror. In some embodiments, the kit further includes one or more finishing formulations. In some embodiments, provided herein is a kit including a therapeutic formulation including a composition provided herein including a catalyst; at least one environment-responsive agent; at least one encapsulating agent; at least one unsaturated organopolymer; and at least one hydride functionalized polysiloxane. In some embodiments, the kit further includes instructions for use of the kit, one or more brushes, one or more swabs, a film removing cleanser or a mirror. In some embodiments, the kit further includes one or more finishing formulations. In some embodiments, provided herein is a kit including a therapeutic formulation including a composition provided herein including a catalyst; at least one environment-responsive agent; at least one ligand; at least bifunctional organopolysiloxane polymer. In some embodiments, provided herein is a kit including a therapeutic formulation including a composition provided herein including a catalyst; at least one environment-responsive agent; at least one encapsulating agent; at least one bifunctional organopolysiloxane polymer. In some embodiments, the kit further includes instructions for use of the kit, one or more brushes, one or more swabs, a film removing cleanser or a mirror. In some embodiments, the kit further includes one or more finishing formulations.

In some embodiments, provided herein is a kit including a therapeutic formulation including a composition provided herein including a catalyst; at least one volatile agent; at least one ligand; at least one vinyl functionalized organopolysiloxane; and at least one hydride functionalized polysiloxane. In some embodiments, the kit further includes instructions for use of the kit, one or more brushes, one or more swabs, a film removing cleanser or a mirror. In some embodiments, the kit further includes one or more finishing formulations. In some embodiments, provided herein is a kit including a therapeutic formulation including a composition provided herein including a catalyst; at least one volatile agent; at least one encapsulating agent; at least one vinyl functionalized organopolysiloxane; and at least one hydride functionalized polysiloxane. In some embodiments, the kit further includes instructions for use of the kit, one or more brushes, one or more swabs, a film removing cleanser or a mirror. In some embodiments, the kit further includes one or more finishing formulations. In some embodiments, provided herein is a kit including a therapeutic formulation including a composition provided herein including a catalyst; at least one volatile agent; at least one ligand; at least bifunctional organopolysiloxane polymer. In some embodiments, provided herein is a kit including a therapeutic formulation including a composition provided herein including a catalyst; at least one volatile agent; at least one encapsulating agent; at least one bifunctional organopolysiloxane polymer. In some embodiments, the kit further includes instructions for use of the kit, one or more brushes, one or more swabs, a film removing cleanser or a mirror. In some embodiments, the kit further includes one or more finishing formulations.

In some embodiments, provided herein is a kit including a composition provided herein including a catalyst; at least one environment-responsive agent; at least one ligand; at least one unsaturated organopolymer; and at least one hydride functionalized polysiloxane, wherein the catalyst catalyzes an in situ cross-linking of the at least one unsaturated organopolymer; and at least one hydride functionalized polysiloxane such that a film is formed on the skin. In some embodiments, provided herein is a kit for repairing a cosmetic film in which the kit includes a composition provided herein including a catalyst; at least one environment-responsive agent; at least one ligand; at least one unsaturated organopolymer; and at least one hydride functionalized polysiloxane wherein the catalyst catalyzes an in situ cross-linking of the at least one unsaturated organopolymer; and at least one hydride functionalized polysiloxane such that a film is formed on the skin. In some embodiments, provided herein is a kit for repairing a therapeutic film in which the kit includes a composition provided herein including a catalyst; at least one environment-responsive agent; at least one ligand; at least one unsaturated organopolymer; and at least one hydride functionalized polysiloxane wherein the catalyst catalyzes an in situ cross-linking of the at least one unsaturated organopolymer; and at least one hydride functionalized polysiloxane such that a film is formed on the skin. In some embodiments, provided herein is a kit including a composition provided herein including a catalyst; at least one environment-responsive agent; at least one ligand; at least one v bifunctional organopolysiloxane polymer, wherein the catalyst catalyzes an in situ hydrosilylation step-growth polymerization reaction of the bifunctional organopolysiloxane polymer such that a film is formed on the skin. In some embodiments, provided herein is a kit for repairing a cosmetic film in which the kit includes a composition provided herein including a catalyst; at least one environment-responsive agent; at least one ligand; at least bifunctional organopolysiloxane polymer wherein the catalyst catalyzes an in situ hydrosilylation step-growth polymerization reaction of the bifunctional organopolysiloxane polymer; such that a film is formed on the skin. In some embodiments, provided herein is a kit for repairing a therapeutic film in which the kit includes a composition provided herein including a catalyst; at least one environment-responsive agent; at least one ligand; at least one bifunctional organopolysiloxane polymer wherein the catalyst catalyzes an in situ hydrosilylation step-growth polymerization reaction of the bifunctional organopolysiloxane polymer such that a film is formed on the skin.

In some embodiments, provided herein is a kit including a composition provided herein including a catalyst; at least one volatile agent; at least one ligand; at least one vinyl functionalized organopolysiloxane; and at least one hydride functionalized polysiloxane, wherein the catalyst catalyzes an in-situ cross-linking of the at least one vinyl functionalized organopolysiloxane; and at least one hydride functionalized polysiloxane such that a film is formed on the skin. In some embodiments, provided herein is a kit for repairing a cosmetic film in which the kit includes a composition provided herein including a catalyst; at least one volatile agent; at least one ligand; at least one vinyl functionalized organopolysiloxane; and at least one hydride functionalized polysiloxane wherein the catalyst catalyzes an in situ cross-linking of the at least one vinyl functionalized organopolysiloxane; and at least one hydride functionalized polysiloxane such that a film is formed on the skin. In some embodiments, provided herein is a kit for repairing a therapeutic film in which the kit includes a composition provided herein including a catalyst; at least one volatile agent; at least one ligand; at least one vinyl functionalized organopolysiloxane; and at least one hydride functionalized polysiloxane wherein the catalyst catalyzes an in situ cross-linking of the at least one vinyl functionalized organopolysiloxane; and at least one hydride functionalized polysiloxane such that a film is formed on the skin. In some embodiments, provided herein is a kit including a composition provided herein including a catalyst; at least one volatile agent; at least one ligand; at least one v bifunctional organopolysiloxane polymer, wherein the catalyst catalyzes an in situ hydrosilylation step-growth polymerization reaction of the bifunctional organopolysiloxane polymer such that a film is formed on the skin. In some embodiments, provided herein is a kit for repairing a cosmetic film in which the kit includes a composition provided herein including a catalyst; at least one volatile agent; at least one ligand; at least bifunctional organopolysiloxane polymer wherein the catalyst catalyzes an in situ hydrosilylation step-growth polymerization reaction of the bifunctional organopolysiloxane polymer; such that a film is formed on the skin. In some embodiments, provided herein is a kit for repairing a therapeutic film in which the kit includes a composition provided herein including a catalyst; at least one volatile agent; at least one ligand; at least one bifunctional organopolysiloxane polymer wherein the catalyst catalyzes an in situ hydrosilylation step-growth polymerization reaction of the bifunctional organopolysiloxane polymer such that a film is formed on the skin.

In some embodiments, provided herein is a kit including a composition provided herein including a catalyst; at least one environment-responsive agent; at least one encapsulating agent; at least one unsaturated organopolymer; and at least one hydride functionalized polysiloxane, wherein the catalyst catalyzes an in-situ cross-linking of the at least one unsaturated organopolymer; and at least one hydride functionalized polysiloxane such that a film is formed on the skin. In some embodiments, provided herein is a kit for repairing a cosmetic film in which the kit includes a composition provided herein including a catalyst; at least one environment-responsive agent; at least one encapsulating agent; at least one unsaturated organopolymer; and at least one hydride functionalized polysiloxane wherein the catalyst catalyzes an in situ cross-linking of the at least one unsaturated organopolymer; and at least one hydride functionalized polysiloxane such that a film is formed on the skin. In some embodiments, provided herein is a kit for repairing a therapeutic film in which the kit includes a composition provided herein including a catalyst; at least one environment-responsive agent; at least one encapsulating agent; at least one unsaturated organopolymer; and at least one hydride functionalized polysiloxane wherein the catalyst catalyzes an in situ cross-linking of the at least one unsaturated organopolymer; and at least one hydride functionalized polysiloxane such that a film is formed on the skin. In some embodiments, provided herein is a kit including a composition provided herein including a catalyst; at least one environment-responsive agent; at least one encapsulating agent; at least one bifunctional organopolysiloxane polymer, wherein the catalyst catalyzes an in situ hydrosilylation step-growth polymerization reaction of the bifunctional organopolysiloxane polymer such that a film is formed on the skin. In some embodiments, provided herein is a kit for repairing a cosmetic film in which the kit includes a composition provided herein including a catalyst; at least one environment-responsive agent; at least one encapsulating agent; at least one bifunctional organopolysiloxane polymer wherein the catalyst catalyzes an in situ hydrosilylation step-growth polymerization reaction of the bifunctional organopolysiloxane polymer such that a film is formed on the skin. In some embodiments, provided herein is a kit for repairing a therapeutic film in which the kit includes a composition provided herein including a catalyst; at least one environment-responsive agent; at least one encapsulating agent; at least one bifunctional organopolysiloxane polymer wherein the catalyst catalyzes an in-situ c hydrosilylation step-growth polymerization reaction of the bifunctional organopolysiloxane polymer such that a film is formed on the skin.

In some embodiments, provided herein is a kit including a composition provided herein including a catalyst; at least one volatile agent; at least one encapsulating agent; at least one vinyl functionalized organopolysiloxane; and at least one hydride functionalized polysiloxane, wherein the catalyst catalyzes an in-situ cross-linking of the at least one vinyl functionalized organopolysiloxane; and at least one hydride functionalized polysiloxane such that a film is formed on the skin. In some embodiments, provided herein is a kit for repairing a cosmetic film in which the kit includes a composition provided herein including a catalyst; at least one volatile agent; at least one encapsulating agent; at least one vinyl functionalized organopolysiloxane; and at least one hydride functionalized polysiloxane wherein the catalyst catalyzes an in situ cross-linking of the at least one vinyl functionalized organopolysiloxane; and at least one hydride functionalized polysiloxane such that a film is formed on the skin. In some embodiments, provided herein is a kit for repairing a therapeutic film in which the kit includes a composition provided herein including a catalyst; at least one volatile agent; at least one encapsulating agent; at least one vinyl functionalized organopolysiloxane; and at least one hydride functionalized polysiloxane wherein the catalyst catalyzes an in situ cross-linking of the at least one vinyl functionalized organopolysiloxane; and at least one hydride functionalized polysiloxane such that a film is formed on the skin. In some embodiments, provided herein is a kit including a composition provided herein including a catalyst; at least one volatile agent; at least one encapsulating agent; at least one bifunctional organopolysiloxane polymer, wherein the catalyst catalyzes an in situ hydrosilylation step-growth polymerization reaction of the bifunctional organopolysiloxane polymer such that a film is formed on the skin. In some embodiments, provided herein is a kit for repairing a cosmetic film in which the kit includes a composition provided herein including a catalyst; at least one volatile agent; at least one encapsulating agent; at least one bifunctional organopolysiloxane polymer wherein the catalyst catalyzes an in situ hydrosilylation step-growth polymerization reaction of the bifunctional organopolysiloxane polymer such that a film is formed on the skin. In some embodiments, provided herein is a kit for repairing a therapeutic film in which the kit includes a composition provided herein including a catalyst; at least one volatile agent; at least one encapsulating agent; at least one bifunctional organopolysiloxane polymer wherein the catalyst catalyzes an in-situ c hydrosilylation step-growth polymerization reaction of the bifunctional organopolysiloxane polymer such that a film is formed on the skin.

Unless otherwise specified, all properties of compositions, layers and/or devices disclosed herein are measured at room temperature (about 22 to 25° C.) and about 1 atmosphere air pressure.

### 1.18 Controlled Delivery Out Of The Environment-Responsive Agent

In one embodiment, the environment-responsive agent is transported out in a controlled manner. In one embodiment, about 10% by weight of the environment-responsive agent is transported out of the environment-responsive agent in 1 hour. In one embodiment, about 15% by weight of the environment-responsive agent is transported out of the environment-responsive agent in 3 hours. In one embodiment, about 20% by weight of the environment-responsive agent is transported out of the environment-responsive agent in 6 hours. In one embodiment, about 40% by weight of the environment-responsive agent is transported out of the environment-responsive agent in 12 hours. In one embodiment, about 60% by weight of the environment-responsive agent is transported out of the environment-responsive agent in 24 hours. In one embodiment, about 80% by weight of the environment-responsive agent is transported out of the environment-responsive agent in 48 hours.

In one embodiment, the environment-responsive agent is transported out in a controlled manner. In one embodiment, about 80% by weight of the environment-responsive agent is transported out of the film in 72 hours. In one embodiment, about 80% by weight of the environment-responsive agent is transported out of the film in 4 days. In one embodiment, about 80% by weight of the environment-responsive agent is transported out of the film in 5 days. In one embodiment, about 80% by weight of the environment-responsive agent is transported out of the film in 6 days. In one embodiment, about 80% by weight of the environment-responsive agent is transported out of the film in 1 week. In one embodiment, about 80% by weight of the environment-responsive agent is transported out of the film in 8 days. In one embodiment, about 80% by weight of the environment-responsive agent is transported out of the film in 9 days. In one embodiment, about 80% by weight of the environment-responsive agent is transported out of the film in 10 days. In one embodiment, about 80% by weight of the environment-responsive agent is transported out of the film in 11 days. In one embodiment, about 80% by weight of the environment-responsive agent is transported out of the film in 12 days. In one embodiment, about 80% by weight of the environment-responsive agent is transported out of the film in 13 days. In one embodiment, about 80% by weight of the environment-responsive agent is transported out of the film in 2 weeks.

In one embodiment, the environment-responsive agent is transported out in about 1 part per trillion per second by weight. In one embodiment, the environment-responsive agent is transported out from the composition into the surrounding atmosphere in about 1 part per billion per second by weight. In one embodiment, the volatile agent is transported out from the composition into the surrounding atmosphere in about 1 part per million per second by weight. In one embodiment, the environment-responsive agent is transported out from the composition into the surrounding atmosphere in about 0.01% by weight. In one embodiment, the environment-responsive agent is transported out from the composition into the surrounding atmosphere in about 0.1% by weight. In one embodiment, the environment-responsive agent is transported out from the composition into the surrounding atmosphere in about 1% by weight. In one embodiment, the environment-responsive agent is transported out from the composition into the surrounding atmosphere in about 10% by weight. In one embodiment, the amount of the transferred-out environment-responsive agent is determined by gas chromatography (GC). In one embodiment, the amount of the transferred-out environment-responsive agent is determined by inductively-coupled plasma (ICP).

### 1.19 Properties Of A Film Created By The Compositions And Methods Provided Herein

In one embodiment, the film formed by the composition provided herein remains substantially intact on said skin for about 24 hours or more.

In one embodiment, the film formed by the composition provided herein remains substantially intact on said skin for about 24 hours or more with routine daily activities and/or with demanding activities.

In one embodiment, the film formed by the composition provided herein remains at least about 50% intact, at least about 60% intact, at least about 70% intact, at least about 80% intact, at least about 90% intact, or at least about 95% intact by either area or by weight on said skin for about 24 hours or more with routine daily activities and/ or with demanding activities.

In one embodiment, the film formed by the composition provided herein remains substantially intact on said skin for more than about 24 hours, more than about 30 hours, more than about 36 hours, more than about 48 hours, more than about 60 hours, more than about 72 hours, more than about 84 hours, more than about 96 hours, more than about 120 hours, more than about 144 hours, or more than about 168 hours with routine daily activities and/or with demanding activities.

In one embodiment, the film formed by the composition provided herein remains at least about 50% intact, at least about 60% intact, at least about 70% intact, at least about 80% intact, at least about 90% intact, or at least about 95% intact by either area or by weight on said skin for more than about 24 hours, more than about 30 hours, more than about 36 hours, more than about 48 hours, more than about 60 hours, more than about 72 hours, more than about 84 hours, more than about 96 hours, more than about 120 hours, more than about 144 hours, or more than about 168 hours with routine daily activities and/or with demanding activities.

In one embodiment, the film formed by the composition provided herein remains substantially intact on said skin for more than about 24 hours, more than about 30 hours, more than about 36 hours, more than about 48 hours, more than about 60 hours, more than about 72 hours, more than about 84 hours, more than about 96 hours, more than about 120 hours, more than about 144 hours, or more than about 168 hours with routine daily activities and/or with demanding activities as determined by the Film Durability on Skin Test.

In one embodiment, the film formed by the composition provided herein remains at least about 50% intact, at least about 60% intact, at least about 70% intact, at least about 80% intact, at least about 90% intact, or at least about 95% intact by either area or by weight on said skin for more than about 24 hours, more than about 30 hours, more than about 36 hours, more than about 48 hours, more than about 60 hours, more than about 72 hours, more than about 84 hours, more than about 96 hours, more than about 120 hours, more than about 144 hours, or more than about 168 hours with routine daily activities and/or with demanding activities as determined by the Film Durability on Skin Test.

In one embodiment, the film formed by the composition provided herein has a set-to-touch time of greater than about 30 seconds and less than about 7 minutes, greater than about 30 seconds and less than about 4 minutes, greater than about 30 seconds and less than about 2 minutes, or of about 2 minutes.

In one embodiment, the film formed by the composition provided herein has a set-to-touch time of greater than about 30 seconds and less than about 7 minutes, greater than about 30 seconds and less than about 4 minutes, greater than about 30 seconds and less than about 2 minutes, or of about 2 minutes, as determined by the Set-to-Touch Time of Film Test.

In certain embodiments, the composition has a tack-free time of greater than about 1 second and less than about 10 minutes. In preferred embodiments, the composition has a tack-free time of greater than about 30 seconds and less than about 4 minutes. In further preferred embodiments, the composition has a tack-free time of greater than about 30 seconds and less than about 2 minutes. In further preferred embodiments, the composition has a tack-free time of greater than about 1 minute and less than about 2 minutes. In other preferred embodiments, the composition has a tack-free time of about 1.5 minutes. Polyurethane and polypropylene have surface conditions preferably used for the measurement of tack-free time due to their smooth surface with low aspect ratio and cure characters in-vitro that are similar to the cure characters on skin in-vivo.

In one embodiment, the film formed by the composition provided herein has an average thickness of less than about 1000 microns, less than about 100 microns, of about 0.5 to about 100 microns, about 1 to about 90 microns, about 10 to about 80 microns, about 30 to about 70 microns, about 40 to about 60 microns, or about 50 microns.

In one embodiment, the film formed by the composition provided herein has an average thickness of less than about 1000 microns, less than about 100 microns, of about 0.5 to about 100 microns, about 1 to about 90 microns, about 10 to about 80 microns, about 30 to about 70 microns, about 40 to about 60 microns, or about 50 microns, as determined by the ASTM D3767 test using Cowhide Tooling leather.

In one embodiment, the film formed in vitro by said composition has a leather adhesive force of greater than about 30 N/mm, greater than about 60 N/mm, greater than about 80 N/mm, greater than about 100 N/mm, or greater than 200 N/mm, as determined by the Leather Peel Adhesion Test.

Another aspect provided herein is directed to a composition that forms a layer on the skin that resists peeling. Resistance to peeling is determined by measuring adhesive force using the Peel Adhesion test described herein. In preferred embodiment, the adhesive force of the layer on polypropylene substrate is greater than about is greater than about 5 N/m. In further preferred embodiments, the adhesive force of the layer on polypropylene substrate is greater than about 20 N/m, 40 N/m, 60 N/m, 80 N/m, greater than about 100 N/m, or greater than about 200 N/m.

In one embodiment, the film formed in vitro by said composition, upon exposure of said test film to environmental factors selected from: heat, cold, wind, water, humidity, bodily fluids, blood, pus/liquor puris, urine, saliva, sputum, tears, semen, milk, vaginal secretion, sebum, saline, seawater, soapy water, detergent water, or chlorinated water, or a combination thereof, has a weight increase, at a time point between about 1-hour and about 168 hours after formation, of less than about 10%, less than about 5, or less than about 1%, as determined by the ASTM D2765-95 test.

In one embodiment, the film formed in vitro by said composition has a tensile strength greater than about 0.25 MPa, greater than about 0.5 MPa, greater than about 1.0 MPa, or greater than about 2.0 MPa, and in one embodiment, said film has a tensile strength less than about 5 MPa, or in one embodiment, said film has a tensile strength at about 3.0 MPa, as determined by the Cyclic and Extension Pull Test.

In one embodiment, the film formed in vitro by said composition has a fracture strain of greater than about 100%, greater than about 200%, greater than about 400%, greater than about 600%, greater than about 800%, greater than about 1000%, greater than about 1200%, or greater than about 1500%, as determined by the Cyclic and Extension Pull Test.

In one embodiment, the film formed in vitro by said composition has a tensile modulus of about 0.01 to about 40 MPa, about 0.05 to about 20 MPa, about 0.1 to about 10 MPa, about 0.1 to about 5 MPa, about 0.1 to about 1 MPa, about 0.25 to about 0.75 MPa, or at about 0.5 MPa, as determined by the Cyclic and Extension Pull Test.

In one embodiment, the film formed in vitro by said composition has a shear modulus of about 0.05 to about 10 MPa, about 0.1 to about 5 MPa, about 0.1 to about 1 MPa, about 0.25 to about 0.75 MPa, or at about 0.5 MPa, as determined by the Cyclic and Extension Pull Test.

In one embodiment, the film formed in vitro by said composition has a cyclic tensile residual strain of less than about 10%, less than about 5%, less than about 2.5%, less than about 1%, less than about 0.5%, less than about 0.25%, or less than about 0.1%, as determined by the Cyclic and Extension Pull Test.

In one embodiment, the film formed in vitro by said composition has a cyclic tensile hysteresis loss energy of less than about 1 kJ/m³, less than about 0.5, kJ/m³, or less than about 0.2 kJ/m³, as determined by the Cyclic and Extension Pull Test.

In one embodiment, the film formed in vitro by said composition has a fracture toughness of greater than about 500 kJ/m³, greater than about 5,000 kJ/m³, greater than about 10,000 kJ/m³, or greater than about 50,000 kJ/m³, as determined by the Cyclic and Extension Pull Test.

In one embodiment, the film formed in vitro by said composition has an oxygen transmission rate of greater than about 5×10⁻⁹ cm³/(cm² s), greater than about 5×10⁻⁷ cm ³/(cm² s), greater than about 5×10⁻⁵ cm³/(cm² s), greater than about 5×10⁻⁴ cm³/(cm² s), greater than about 5×10⁻³ cm³/(cm² s), greater than about 5×10⁻² cm³/(cm² s), or greater than about 0.5 cm³/(cm² s), and in one embodiment, said film has an oxygen transmission rate of less than about 5 cm³/(cm² s), as determined by the ASTM F2622 test.

In one embodiment, the film formed in vitro by said composition has an oxygen permeance of greater than about 5×10⁻¹¹ cm³/(cm² s cm Hg), greater than about 5×10⁻⁹ cm³/(cm² s cm Hg), greater than about 5×10⁻⁷ cm³/(cm² s cm Hg), greater than about 5×10⁻⁶, 5×10⁻⁵ cm³/(cm² s cm Hg), greater than about 5×10⁻⁴ cm³/(cm² s cm Hg), greater than about 5×10⁻³ cm³/(cm² s cm Hg), greater than about or 5×10⁻² cm³/(cm² s cm Hg), and in one embodiment, said film has an oxygen permeance of less than about 0.5 cm³ / (cm² s cm Hg), as determined by the ASTM F2622 test.

In one embodiment, the film formed in vitro by said composition has an oxygen permeability coefficient of greater than about 5×10⁻⁴ Barrer, greater than about 5×10⁻² Barrer, greater than about 5 Barrer, greater than about 50 Barrer, greater than about 500 Barrer, or greater than about 5,000 Barrer, and in one embodiment, said film has an oxygen permeability coefficient of less than about 20,000 Barrer, as determined by the ASTM F2622 test.

In one embodiment, the film formed in vitro by said composition has a water vapor transmission rate of greater than about 1×10⁻⁹ cm³/(cm² s), greater than about 1×10⁻⁸ cm ³/(cm² s), greater than about 1×10⁻⁷, 1×10⁻⁶ cm³/(cm² s), greater than about 1×10⁻⁵ cm³ / (cm² s), or greater than about 1×10⁻⁴ cm³/(cm² s), and in one embodiment, said film has a water vapor transmission rate of less than about 1.5×10⁻¹ cm³/(cm² s) or less than about 1.5×10⁻² cm³/(cm² s), as determined by the ASTM F1249 test.

In one embodiment, the film formed in vitro by said composition has a water vapor permeance of greater than about 1×10⁻¹¹ cm³/(cm² s cm Hg), greater than about 1×10⁻¹⁰ cm3/(cm² s cm Hg), greater than about 1×10⁻⁹ cm³/(cm² s cm Hg), greater than about 1×10⁻⁸ cm³/(cm² s cm Hg), greater than about 1×10⁻⁷ cm³/(cm² s cm Hg), and in one embodiment, said film has a water vapor permeance of less than about 2×10⁻³ cm³/(cm² s cm Hg) or less than about 2×10⁻² cm³/(cm² s cm Hg), as determined by the ASTM F1249 test.

In one embodiment, the film formed in vitro by said composition has a water vapor permeability coefficient of greater than about 1×10⁻³ Barrer, greater than about 0.01 Barrer, greater than about 0.1 Barrer, greater than about 1 Barrer, greater than about 10 Barrer, greater than about 100 Barrer, greater than about 1×10³ Barrer, or greater than about 1×10⁴ Barrer, and in one embodiment, said film has a water vapor permeability coefficient of less than about 1×10⁶ Barrer or less than about 1×10⁵ Barrer, as determined by the ASTM F1249 test.

In one embodiment, said film has a transepidermal water loss of less than about 40 g/ (m² hr), less than about 20 g/(m² hr), less than about 10 g/(m² hr), less than about 5 g/ (m² hr), or less than about 1 g/(m² hr), as determined by Transepidermal Water Loss (TEWL) Measurement Test using an evaporimeter at a time point between about 1-hour and about 168 hours after application of the composition.

In one embodiment, said film has a skin hydration of greater than about 20 arbitrary units, greater than about 40 arbitrary units, greater than about 60 arbitrary units, or greater than about 80 arbitrary units of Corneometer, as determined by the Dobrev method using a Corneometer at a time point between about 1-hour and about 168 hours after application of the composition.

In one embodiment, said film has a skin hydration of greater than about 20 microSiemens, greater than about 50 microSiemens, greater than about 100 microSiemens, greater than about 200 microSiemens, or greater than about 400 microSiemens, as determined by the Clarys method using a Conductance or Impedance Meter at a time point between about 1-hour and about 168 hours after application of the composition.

In one embodiment, said film has a skin retraction time decreased by about 5%, decreased by about 10%, decreased by about 25%, decreased by about 50%, or decreased by about 75%, as determined by the Dobrev method using a Cutometer or Suction Cup at a time point between about 1-hour and about 168 hours after application of the composition.

In one embodiment, the film formed in vitro by said composition has a shine and/or gloss change of the area treated with said composition of less than about 20%, less than about 10%, or less than about 5%, as determined by the ASTM D523 test using Cowhide Tooling leather in natural color as substrate.

In one embodiment, the film formed in vitro by said composition has a color L* scale change of the area treated with said composition of less than about 2, less than about 1.5, less than about 1, or less than about 0.5, as determined by the ASTM E313 test using Cowhide Tooling leather in natural color as substrate.

In one embodiment, the film formed in vitro by said composition has a color a* scale change of the area treated with said composition of less than about 2, less than about 1.5, less than about 1, or less than about 0.5, as determined by the ASTM E313 test using Cowhide Tooling leather in natural color as substrate.

In one embodiment, the film formed in vitro by said composition has a color b* scale change of the area treated with said composition of less than about 2, less than about 1.5, less than about 1, or less than about 0.5, as determined by the ASTM E313 test using Cowhide Tooling leather in natural color as substrate.

In one embodiment, the film formed in vitro by said composition has a tensile strength between about 0.01 MPa and about 10 MPa, as determined by the Cyclic and Extension Pull Test.

In one embodiment, the film formed in vitro by said composition has a tensile strength between about 0.1 MPa and about 2.5 MPa, as determined by the Cyclic and Extension Pull Test.

In one embodiment, the film formed in vitro by said composition has a fracture strain between about 10% and about 1500%, as determined by the Cyclic and Extension Pull Test.

In one embodiment, the film formed in vitro by said composition has a fracture strain between about 10% and about 600%, as determined by the Cyclic and Extension Pull Test.

In one embodiment, the film formed in vitro by said composition has a tensile modulus between about 0.01 and about 10 MPa, as determined by the Cyclic and Extension Pull Test.

In one embodiment, the film formed in vitro by said composition has a tensile modulus between about 0.01 and about 2.5 MPa, as determined by the Cyclic and Extension Pull Test.In one embodiment, the film formed in vitro by said composition has a cyclic tensile residual strain between about 0.1% and about 10%, as determined by the Cyclic and Extension Pull Test.

In one embodiment, the film formed in vitro by said composition has a cyclic tensile residual strain between about 0.1% and about 5%, as determined by the Cyclic and Extension Pull Test.

In one embodiment, the film formed in vitro by said composition has a cyclic tensile hysteresis loss energy between about 0.01 kJ/m³ and about 1 kJ/m³, as determined by the Cyclic and Extension Pull Test.

In one embodiment, the film formed in vitro by said composition has a cyclic tensile hysteresis loss energy between about 0.05 kJ/m³ and about 0.5 kJ/m³, as determined by the Cyclic and Extension Pull Test.

In one embodiment, the film formed in vitro by said composition has a fracture toughness between about 500 kJ/m³ and about 50,000 kJ/m³, as determined by the Cyclic and Extension Pull Test.

In one embodiment, the film formed in vitro by said composition has a fracture toughness between about 1,000 kJ/m³ and about 12,000 kJ/m³, as determined by the Cyclic and Extension Pull Test.

In one embodiment, the film formed in vitro by said composition has an oxygen transmission rate of about 0.5 cm³/(cm² s), as determined by the ASTM F2622 test.

In one embodiment, the film formed in vitro by said composition has an oxygen transmission rate of greater than about 0.18 cm³/(cm² s), as determined by the ASTM F2622 test.

In one embodiment, the film formed in vitro by said composition has an oxygen permeance of about 0.005 cm³/(cm² s cm Hg), as determined by the ASTM F2622 test.

In one embodiment, the film formed in vitro by said composition has an oxygen permeance of greater than about 0.002 cm³/(cm² s cm Hg), as determined by the ASTM F2622 test.

In one embodiment, the film formed in vitro by said composition has an oxygen permeability coefficient of about 3.5×10⁵ Barrer, as determined by the ASTM F2622 test.

In one embodiment, the film formed in vitro by said composition has an oxygen permeability coefficient of greater than about 1.4×10⁵ Barrer, as determined by the ASTM F2622 test.

In one embodiment, the film formed in vitro by said composition has a water vapor transmission rate of about 5×10⁻⁴ cm³/(cm² s), as determined by the ASTM F1249 test.

In one embodiment, the film formed in vitro by said composition has a water vapor transmission rate of greater than about 5×10⁻⁵ cm³/(cm² s), as determined by the ASTM F1249 test.

In one embodiment, the film formed in vitro by said composition has a water vapor permeance of about 5×10⁻⁶ cm³/(cm² s cm Hg), as determined by the ASTM F1249 test.

In one embodiment, the film formed in vitro by said composition has a water vapor permeance of greater than about 5×10⁻⁷ cm³/(cm² s cm Hg), as determined by the ASTM F1249 test.

In one embodiment, the film formed in vitro by said composition has a water vapor permeability coefficient of about 350 Barrer, as determined by the ASTM F1249 test.

In one embodiment, the film formed in vitro by said composition has a water vapor permeability coefficient of greater than about 35 Barrer, as determined by the ASTM F1249 test.

### 1.20 Assays For Use With The Compositions And Methods Provided Herein

In certain embodiments, a film resulting from a composition described herein, e.g., by applying the composition to the skin of a subject has specified properties. The following assays can be used to demonstrate the properties of the film generated with the composition and methods provided herein.

### 1.20.1 Rheometer Viscosity Measurement Test

The following test method may be used to determine the dynamic viscosity (Pas) of fluid materials at 0.5 s⁻¹, using a Bohlin CVO100 Rheometer (Malvern Instruments) mounted with 20 mm Parallel plate geometry. Similar Rheometers can be used for viscosity measurements. For each material tested, at least 3 samples are measured, and average viscosity and standard deviation of the measurements are recorded.

About 1 g of each test sample is required. Visually inspect the sample to ensure the sample appears uniform. Turn on the Bohlin Rheometer and the temperature controller; start the Bohlin software and load the viscosity stability test template; install the geometry and zero the instrument. Make sure that both the geometry and plate are clean, which is critical for accurate test results. Place about 1 g of the test sample onto the bottom plate of the Rheometer in a mound centered below the geometry. Lower the geometry to the correct gap (about 250 µm). Clean any excess sample from the sides of the geometry using the flat end of a spatula. Start the test and allow the test to run to completion, then record the viscosity (Pas) data.

In certain embodiments, a film generated with the compositions and methods provided herein has particular dynamic viscosity. In certain embodiments, the dynamic viscosity can be determined using the assay of the Rheometer Viscosity Measurement Test provided herein.

### 1.20.2 Film Durability on Skin Test

### Application of Test Composition

Healthy subjects (at least 3) are selected irrespective of age, race or gender. Tests are conducted at room temperature and about 50% relative humidity. Drawn 4 ×4 cm² square outlines on selected volar forearm areas using a standard template as guide. Using a balance, weigh out appropriate amounts (e.g., about 0.1 g to about 0.3 g) of the test composition (or about 0.1 g of the first part and about 0.15 g of the second part in cases of a two-part composition) onto weigh boats (in cases of a two-part composition, do not mix). Apply the test composition evenly over the 4 ×4 cm² squares on the forearm using a fingertip, preferably wearing finger cot. Make sure that all areas of the squares are covered by the composition. In case of a two-part composition, a clean fingertip or fresh finger cot should be used to spread the second part gently over the first part, covering the entire first part area.

### Measurement

The composition is allowed to sit untouched over the area for about 15 minutes. The subject is then allowed to resume daily activities. The subjects are permitted to conduct either only routine daily activities, or routine daily activities with demanding activities, for example, exercising, swimming, steam room, sauna, and the like. The type and length of each demanding activity are recorded. The layers formed by the test composition are left on skin for about 24 hours or more. At certain time points after application of the composition, durability of layers are assessed by measuring the percentage of the area intact on the skin using an 8×8 square grid of 0.5×0.5 cm² each (total 64 squares). Any excess layer outside of the 4 ×4 cm² square area is not considered in the evaluation. Each square is only considered to be durable if there is no visible imperfection, e.g., seams, flaking, cracking, and/or peeling, of the layer. Record the observations.

In certain embodiments, a film generated with the compositions and methods provided herein has particular film durability. In certain embodiments, the film durability can be determined using the assay of the Film Durability on Skin Test provided herein.

### 1.20.3 Set-to-Touch Time and Tack-Free Time of Film Test

This method was modified from ASTM D5895-03 Evaluating Drying or Curing During Film Formation of Organic Coatings Using Mechanical Recorders. The materials and application of test composition to the selected subjects are the same as described in the Film Durability on Skin Test. The test can also be conducted on other substrates instead of human skin, for example, on Cowhide Tooling leather in natural color, polyurethane, or polypropylene substrates with comparable results. For each composition tested, at least 3 samples are tested, and average set-to-touch time, average tack-free time and standard deviation of the measurements are recorded.

### Measurement

Start a timer when the test composition (or the second part in case of a two-part composition) is applied to the entire test area on the forearm. Allow the composition to sit untouched over the area for a certain period of time, e.g., 30 seconds or one minute. At certain time points, touch one corner of the test area lightly using a fingertip, and visually evaluate: first the presence or absence of any test composition on the fingertip (Set-to-Touch Time); then the presence or absence of any film surface being pulled up by the fingertip (Tack-Free Time of Film Test). Repeat the fingertip evaluation on untouched portions of the test area at a certain time interval, e.g., every 15 seconds or 30 seconds or one minute. The time at which no more test composition is observed on the fingertip is reported as the “set-to-touch time” of the test composition. The time at which no more film surface is pulled up by the fingertip is reported as the “tack-free time” of the test composition.

In certain embodiments, a film generated with the compositions and methods provided herein has particular set-to-touch time and tack-free time. In certain embodiments, the set-to-touch time and tack-free time can be determined using the assay of the Set-to-Touch Time and Tack-Free Time of Film Test provided herein.

### 1.20.4 Set-to-Touch Time and Tack-Free Time of Film Test In-Vitro

This method was modified from ASTM D5895-03 Evaluating Drying or Curing During Film Formation of Organic Coatings Using Mechanical Recorders. The materials and application of test composition to the selected substrates are described as follows: Place a 50-micron spacer (for example, one layer of 3 M Magic Scotch Tape) onto the substrate sheet size 4.5″ × 1.5″, forming an opening rectangular of 3.75″ × 0.75″, exposing the substrate surface. Apply test composition onto the substrate, then gliding the glass slide back and forth along the spacer edges to deposit a smooth and uniform layer of test composition. The test can also be conducted on many substrates such as on Cowhide Tooling leather in natural color, polyurethane, or polypropylene substrates with comparable results. For each composition tested, at least 3 samples are tested, and average set-to-touch time, average tack-free time and standard deviation of the measurements are recorded.

### Measurement

Start a timer when the test composition (or the second part in case of a two-part composition) is applied to the entire test area on the substrate. Allow the test composition to sit untouched over the area at room temperature and ambient humidity for a certain period of time, e.g., 30 seconds or one minute. At certain time points, place a 1.5 cm x 4 cm polypropylene sheet on the surface of the test composition, then place a 15 g weight on top of polypropylene sheet. Wait for 2 seconds, before removing the weight and the polypropylene sheet from the surface of the test composition. Visually evaluate: first the presence or absence of any test composition on the polypropylene sheet. Repeat the polypropylene sheet evaluation on untouched portions of the test area at a certain time interval, e.g., every 15 seconds or 30 seconds or one minute. The time at which no more test composition on the polypropylene sheet is observed is reported as the “set-to-touch time” of the test composition. After “set-to-touch time” is reported, transfer the specimen to the 30-degree slope surface to evaluate the “tack-free time”. Place the specimen 6 inches up along the slope surface away from the lowest point and secure the specimen on the slope surface. Drop a 1/32″ diameter stainless steel ball onto the top part of the film surface from a distance an inch above the film surface. Observe the movement of the stainless steel ball on the film surface as the ball trying to roll down on its own gravity. Report “tack-free time” when the ball is able to roll from the top to the bottom part of the film surface continuously, without any interruption from the frictional film surface as the film becomes tack-free.

In certain embodiments, a film generated with the compositions and methods provided herein has particular set-to-touch time and tack-free time. In certain embodiments, the set-to-touch time and tack-free time can be determined using the assay of the Set-to-Touch Time and Tack-Free Time of Film Test in-vitro provided herein.

### 1.20.5 Peel Adhesion Test

This test method for adhesive force was developed in accordance with ASTM C794 Adhesion-in-Peel of Elastomeric Joint Sealants. Instron 3342 single column tension/ compression testing system (Instron, Norwood, MA) with 100 N load cell (Instron #2519-103) mounted with extension grip geometry may be used, with polypropylene sheet of 1/32″ thickness as test substrate. Other similar equipment and other soft, flexible test substrates can also be used to measure the peeling force. The materials and application of test composition to the selected substrates are described as follows: Place a 50-micron spacer (for example, one layer of 3 M Magic Scotch Tape) onto the substrate sheet size 4.5″ × 1.5″, forming an opening rectangular of 3.75″ × 0.75″, exposing the substrate surface. Apply test composition onto the substrate, then gliding the glass slide back and forth along the spacer edges to deposit a smooth and uniform layer of test composition. Allow the test composition to sit untouched over the area at room temperature and ambient humidity for 24 hours. Then, place a silicone adhesive tape (Mepitac) of 0.75″ width on top of the film to fully cover the film surface on the polypropylene substrate, wait at room temperature and ambient humidity for 24 hours before the specimen is ready for measurement. For each material tested, at least 3 samples are measured, and average peeling force and standard deviation of the measurements are recorded.

### Measurement

Partially peel the silicone tape-covered test specimen at one end by hand to separate enough of the silicone tape-covered film from the polypropylene substrate for effective grip by extension grip geometry mounts of the instrument. Secure each peeling side in its own instrument grip. Make sure the strips are clamped substantially parallel to the geometry. Perform the extension test at a rate of 1 mm/s until the two peeling strips separate completely from each other. Record the peeling force vs. time data. The sample’s average peeling force (N/m) is calculated by averaging the instantaneous force (N) measured by the instrument during the experiment normalized by the sample width (0.75″ or 0.019 m).

In certain embodiments, a film generated with the compositions and methods provided herein has particular adhesive force. In certain embodiments, the adhesive force can be determined using the assay of the Peel Adhesion Test provided herein.

### 1.20.6 Curl Test for Tension of Curved Specimen

The deposition of the test article on substrate such as skin or elastic band or parafilm results in residual compressive stress within the film due to volume loss (strain), which in turn translate to the tensile stress on the underneath substrate. The combined result of the film deposited on substrate could be observed and quantified based on the level of surface curvature of the substrate after the deposition of the film.

To prepare the test article for curl test, first the test article was deposited onto either an elastic synthetic rubber sheet or a parafilm substrate as described earlier in the application of test composition to the selected substrates. The materials and application of test composition to the selected substrates are described as follows: Place a 50-micron spacer (for example, one layer of 3 M Magic Scotch Tape) onto the substrate sheet size 4.5″ × 1.5″, forming an opening rectangular of 3.75″ × 0.75″, exposing the substrate surface. Apply test composition onto the substrate, then gliding the glass slide back and forth along the spacer edges to deposit a smooth and uniform layer of test composition. Allow the test composition to sit untouched over the area at room temperature and ambient humidity for 24 hours.

### Measurement

Use a Vernier Caliper or optical microscope to measure the end-to-end distance of the width side of the test specimen that is curved upward. The end-to-end distance refers to the chord length, forming an incomplete upward circle where subsequent calculation of corresponding radius of the circle is computed. Report the radius value and its reciprocal as the “curvature” value. Use the curvature value to calculate the tension incurred on the substrate. In the case of originally curved surface with inherent tension such as skin, the change in tension incurred by the deposited top layer, will modify the inherent tension accordingly.

In certain embodiments, a film generated with the compositions and methods provided herein has particular tension. In certain embodiments, the tension can be determined using the assay of the Curl Test for Tension of Curved Specimen provided herein.

### 1.20.7 Cyclic and Extension Pull Test

These test methods for Cyclic Tensile Residual Strain (Instant Residual Strain), Cyclic Tensile Hysteresis Loss Energy, Tensile (Young’s) Modulus, Shear Modulus, Tensile Strength/Maximum Stress, Fracture Strain, and Fracture Toughness was developed to be better suited for the specimens disclosed herein in compliance with ASTM D638, ASTM D412, ASTM D1876 test guidelines. Instron 3342 single column tension/compression testing system (Instron, Norwood, MA) with 100N load cell (Instron #2519-103) mounted with extension grip geometry may be used. Other similar equipment can also be used to measure the properties tested herein. For each material tested, at least 3 samples are measured, and average results and standard deviation of the measurements are recorded.

About 10 g of the composition tested is needed for each sample. The samples are cast inside dumbbell shaped molds mounted on Teflon, consistent with the ASTM D638 guidelines. The dimensions of the “neck” of the mold are about 20 mm in length, about 5 mm in width and about 1.5 mm in depth. The dimensions of the “handles/bell” of the mold are about 20 mm in length, about 15 mm in width and about 1.5 mm in depth, which provides adequate area to insure secure slip-free grip during testing. Level the top surface of the filled mold with a smooth microscope slide. Ensure that the molds are filled without voids and the top surface is smooth. The casted samples are allowed to fully cure and dry for about 20 to about 30 hours. The specimens formed are extracted from their individual molds by means of a spatula. Width and thickness of the “neck” of the finished specimens are measured with a caliper, recorded and input into the instrument. The Area of the “neck” portion of the specimen is calculated by its width and thickness.

Layers formed by compositions disclosed herein can also be tested once separated from the substrates. Such a layer can be formed or trimmed into a rectangular shape, and the Area of a cross-section of a layer can be calculated by its width and thickness. In such as case, the ends of the rectangular specimen would be considered the “handle/bell” portions whereas the middle of the rectangular specimen would be considered the “neck” portion.

An alternative specimen preparation is to place a 50-micron spacer (for example, one layer of 3 M Magic Scotch Tape) onto the polypropylene substrate sheet size 4.5″ × 1.5″, forming an opening rectangular of 3.75″ × 0.75″, exposing the substrate surface. Apply test composition onto the substrate, then gliding the glass slide back and forth along the spacer edges to deposit a smooth and uniform layer of test composition. Allow the test composition to sit untouched over the area at room temperature and ambient humidity for 24 hours.

Mechanical characterization of specimens is carried out on the Instron 3342 (Instron, Norwood MA) equipped with 100N load-cell. Dumbbell or rectangular shaped specimens are mounted onto the instrument via Instron 2710-101 grips on each end, which are modified to insure the specimens do not slip or fail inside the grips during testing. The specimen is mounted onto the instrument such that all the rectangular “handle/bell” portions of the specimen and none of the “neck” of the specimen are fixed within the instrument grips. Make sure that the specimen is mounted substantially vertical in both vertical planes. The instrument grip distance is adjusted such that the sample is at neutral extension as indicated by the instrument force being close to zero (±0.01 N).

Two types of tests are performed sequentially on each specimen, first the Cyclic Test followed by the Extension Pull Test. It is noted that the Cyclic Test has negligible effects on the result of the Extension Pull Test on the same specimen. Each test is preprogrammed into the instrument.

### Cyclic Test

The Cyclic Test is designed to determine the elasticity of the tested materials by measuring Cyclic Tensile Residual Strain (Instant Residual Strain). Generally, the more elastic the material, the faster it returns to its original shape after deformation. Lower Cyclic Tensile Residual Strain scores indicate better elasticity. For perfectly elastic materials, the Cyclic Tensile Residual Strain and cycle test area should approach zero.

The specimen is mounted onto the instrument as described above. Stretch the specimen slightly at about 1 mm/s by raising the geometry until a force of 0.06-0.08 N is registered by the instrument, record the stretched length of the “neck” portion of the specimen as the initial specimen length. Cyclic extension is performed at about 1 mm/s to a maximum extension of 15% of initial specimen length. A total of 15 (and up to 100) cycles are executed and the stress strain data is recorded.

The Cyclic Tensile Modulus is calculated as the straight line slope of the stress-strain curve of first cycle between 1% and 4% strain. The R squared value of the linear fit should be above 0.99 or the test data should be recorded as outlier and discarded. The Cyclic Tensile Residual Strain is calculated for each cycle as the strain difference between the loading and unloading curves at half the maximum stress achieved during the first cycle. The Cyclic Tensile Residual Strain for the first cycle as well as the average Cyclic Tensile Residual Strain for the 2nd through 12th cycles are recorded. The area bound by the loading and unloading curves of each cycle is also calculated as Cyclic Tensile Hysteresis Loss Energy. Good agreement is observed between the Cyclic Tensile Residual Strain and the calculated cycle area.

The majority of the specimens formed by the compositions disclosed herein are sufficiently flexible and elastic such that the Cyclic Test could be repeated on the same sample without a significant change in calculated properties, which suggests that this test did not result in long lasting changes to the tested specimens.

### Extension Pull Test

The Extension Pull Test was used to determine the stiffness and stretchiness/ flexibility of a material by measuring the Tensile/Young’s Modulus and fracture strain, respectively.

The specimen is mounted onto the instrument as described above. Stretch the specimen slightly at about 10 mm/s by raising the geometry until a force of 0.01 to 0.02 N is registered by the instrument, record the stretched length of the “neck” portion of the specimen as “Original Length.” The extension Tensile/Young’s Modulus is calculated as the straight line slope of the stress-strain curve between 6% and 11% strain. The R squared value of the linear fit should be above 0.99 or the Tensile/ Young’s Modulus is calculated from a more linear 5% strain range on the stress strain curve.

The Shear Modulus is determined from the same strain range as the Tensile/Young’s Modulus. Shear Modulus is calculated as the slope of the best line fit between recorded stress and α to 1/α², where α is 1 plus the instantaneous strain.

Stretch the specimen at about 10 mm/s until it is broken at one side or completely. Record the force applied at the time when the specimen is broken as the “Maximum Tensile Force.” Record the length of the “neck” portion of the specimen when it is broken extended beyond the Original Length of the specimen as the “Maximum Elongation Length.” Tensile Strength/Maximum Stress is calculated as the Maximum Tensile Force over the Area of the “neck” portion of the specimen. Fracture Strain is calculated as the Maximum Elongation Length as percentage of the Original Length.

Fracture Toughness (kJ/m³) is calculated as the area under the stress-strain curve in the Extension Pull Test. The Yield Strain is determined as the strain at which the measured stress differed by more than 10% from the Neo-Hookean stress; the multiple of Shear Modulus and (α to 1/α²).

In certain embodiments, a film generated with the compositions and methods provided herein has particular Cyclic Tensile Residual Strain (Instant Residual Strain), Cyclic Tensile Hysteresis Loss Energy, Tensile (Young’s) Modulus, Shear Modulus, Tensile Strength/Maximum Stress, Fracture Strain, and Fracture Toughness. In certain embodiments, the Cyclic Tensile Residual Strain (Instant Residual Strain), Cyclic Tensile Hysteresis Loss Energy, Tensile (Young’s) Modulus, Shear Modulus, Tensile Strength/Maximum Stress, Fracture Strain, and Fracture Toughness can be determined using the assay of the Cyclic and Extension Pull Test provided herein.

### 1.20.8 Transepidermal Water Loss (TEWL) Measurement Test

Evaporative water loss measurements provide an instrumental assessment of skin barrier function. Evaporimetry with TEWL Probe is fully described in Grove et al., Comparative metrology of the evaporimeter and the DermaLab (Registered Trademark) TEWL probe, Skin Res. & Tech. 1999, 5:1-8 and Grove et al., Computerized evaporimetry using the DermaLab (Registered Trademark) TEWL probe, Skin Res. & Tech. 1999, 5:9-13. The guidelines established for using the Servo Med Evaporimeter described by Pinnagoda (Pinnagoda et al., Guidelines for transepidermal water loss (TEWL) measurement, Contact Dermatitis 1990, 22:164-178) are appropriate for the DermaLab (Registered Trademark) TEWL Probe as well.

Evaporative water loss measurements can be made using a recently calibrated Servo Med Evaporimeter. Alternatively, these measurements can be made using a recently calibrated cyberDERM RG1 Evaporimeter System (Broomall, PA) with TEWL Probes (manufactured by Cortex Technology of Hadsund, Denmark and available in the US through cyberDERM, Inc. Broomall, PA), or other similar equipment.

Both Evaporimeters are based on the vapor pressure gradient estimation method pioneered by Gert E. Nilsson (e.g., Nilsson, G.E., Measurement of water exchange through skin, Med Biol Eng Comput 1977, 15:209-218). There are slight dimensional differences and the sensor technology is greatly improved in the DermaLab (Registered Trademark) TEWL Probe but the underlying principles of the measurement remain the same. Both probes contain two sensors that measure the temperature and relative humidity at two fixed points along the axis normal to the skin surface. This arrangement is such that the device can electronically derive a value that corresponds to evaporative water loss expressed in gm/(m² hr). The Evaporimeter System extracts value of average evaporative water loss rate collected over a twenty-second interval once steady state conditions had been achieved.

Subjects are treated with test compositions on selected volar forearm test areas as described in the Film Durability on Skin Test. Measurements are taken from each of the volar forearm sites prior to treatment and at various time points (for example, at about 1-hour, about 4-hour, about 6-hour, about 12-hour, about 24-hour, about 30-hour, about 36-hour, about 48-hour, or between 48 hours and one week time point) after application of the composition. Measurements are taken following a minimum of 25 minutes acclimation period in a controlled environment with the relative humidity maintained at less than about 50% and temperature maintained at about 19 to 22° C. Duplicate water loss readings are taken from each site. TEWL properties (g/(m² hr)) are calculated based on the data recorded by the instrument.

### Optical Measurement Based on Color L*a*b* Test

This test uses a Minolta CR-400 Chroma meter in accordance with the instructions by the manufacturer, which are generally known in the art. Triplicate measurements of L^{∗}(D65), a^{∗}(D65), and b^{∗}(D65) are then collected at 6 or more different locations of the test articles.

### Barrier Protection Test Based on Viral Penetration

Barrier protection test based on viral penetration is performed to evaluate the barrier performance of protective materials, which are intended to protect against blood borne pathogen hazards. Test articles were conditioned for a minimum of 24 hours at 21 ± 5° C. and 60 ± 10% relative humidity (%RH) and then tested for viral penetraton using a ΦX174 bacteriophage suspension. At the end of the test, the observed side of the test article was rinsed with a sterile medium and assayed for the presence of ΦX174 bacteriophage. The viral penetration method complies with ISO 16604. Triplicate readings are taken from each test article.

In certain embodiments, a film generated with the compositions and methods provided herein has particular evaporative water loss. In certain embodiments, the evaporative water loss can be determined using the assay of the Transepidermal Water Loss (TEWL) Measurement Test provided herein.

### 1.20.9 Barrier Protection Test Based On Chemical Protection Against Nickel Contact

Nickel can be detected at the ppm level with a simple spot test containing 1% dimethylglyoxime and 10% ammonium hydroxide solution, which turns pink upon contact with nickel. A 0.2 M solution of nickel (II) sulfate hexahydrate solution is added to a substrate, and both are covered by the test article. The spot test solution is subsequently applied on the test. A change of color to pink indicates that the nickel has penetrated the test article and come in contact with the color solution, or vice versa. In contrast, absence of color change indicates that the test article is not penetrated and that its barrier function is intact.

In certain embodiments, a film generated with the compositions and methods provided herein provides particular barrier protection against nickel contact. In certain embodiments, the barrier protection against nickel contact can be determined using the assay of the barrier protection test based on chemical protection against nickel contact provided herein.

### 1.20.10 Barrier Protection Test Based On Protection from Ultraviolet Radiation

The presence of the test article could help reduce the skin absorption of ultraviolet light, particularly when the test article contains SPF active ingredients such as titanium dioxide, zinc oxide, avobenzone, octinoxate, octocrylene, homosalate, or oxybenzone.

To prepare the test article for barrier protection against UV radiation, first the test article was deposited onto a blank Cellophane sheet substrate as described earlier in the application of test composition to the selected substrates. Cellophane sheet size 12.78 cm(L) x 8.55 cm(W) is employed to match plateholder of UV-Vis Spectrophotometer. Measure UV absorbance with UV-Vis Spectrophotometer from the wavelength 260 nm to 400 nm with 1 nm scan interval. Report absorption data based on averaged value of at least 4 different spot locations.

In certain embodiments, a film generated with the compositions and methods provided herein provides particular barrier protection against UV radiation. In certain embodiments, the barrier protection against UV radiation can be determined using the assay of the barrier protection test based on protection from ultraviolet radiation provided herein.

### EXAMPLES

The testing procedures used in Examples 1, 2, 3, 4 and 5 are described as follows.

### Set-to-Touch Time

The set-to-touch times of the tested formulations were determined in vitro by a modified ASTM D5895-03 method (“Standard Test Methods for Evaluating Drying or Curing during Film Formation of Organic Coatings using Mechanical Recorders”), as described below. These tests mimic the behavior of the tested formulation on skin (referred to herein as “Bioskin”). The test formulation was applied to a sheet of polyurethane substrate with a thickness of about 100 microns, but this thickness was then reduced quickly due to evaporation. The test formulation was allowed to solidify on the substrate at room temperature and ambient humidity until its shine had finished decreasing, as determined by the naked eye. A sheet of porous polypropylene film (Clean & Clear Oil Control Film) (1.5 cm × 4 cm, corresponding to 0.59 inches × 1.57 inches) was then layered carefully on the surface of the test formulation without disturbing it. A weight (15 g; 1 cm wide, 2 cm long and 4.5 cm high, corresponding to 0.39 inches wide, 0.79 inches long and 1.77 inches high) was then placed on top of the polypropylene sheet so that the weight’s side defined by the weight’s length and width made contact with the sample. After two seconds, the weight was removed and the polypropylene sheet was carefully peeled off the test formulation. Then, the polypropylene sheet was inspected visually by naked eye (i.e., without a magnifying device) to determine whether any test formulation was present on it and whether the curing film surface was damaged. This test was repeated about every 15 seconds on areas of the test formulation that had not been subjected to the afore-mentioned weight, using a new polypropylene sheet each time. The time at which no more curing film surface was damaged was observed on the polypropylene sheet was determined to be the in vitro set-to-touch time of the test formulation.

### Bioskin Dry Up Time

The dry up times of the tested formulations were determined in vitro by a modified ASTM D5895-03 method (“Standard Test Methods for Evaluating Drying or Curing during Film Formation of Organic Coatings using Mechanical Recorders”), as described below. These tests mimic the behavior of the tested formulation on skin (i.e., Bioskin). The test formulation was applied to a sheet of polyurethane substrate with a thickness of about 100 microns, but this thickness was then reduced quickly due to evaporation. The test formulation was allowed to solidify on the substrate at room temperature and ambient humidity until its shine had finished decreasing, as determined by naked eye. A sheet of porous polypropylene film (Clean & Clear Oil Control Film) (1.5 cm × 4 cm, corresponding to 0.59 inches × 1.57 inches) was then layered carefully on the surface of the test formulation without disturbing it. A weight (15 g; 1 cm wide, 2 cm long and 4.5 cm high, corresponding to 0.39 inches wide, 0.79 inches long and 1.77 inches high) was then placed on top of the sheet so that the weight’s side defined by the weight’s length and width made contact with the sample. After two seconds, the weight was removed and the sheet was carefully peeled off the test formulation. Then, the sheet was inspected visually by naked eye (i.e., without a magnifying device) to determine whether any test formulation was present on it. This test was repeated about every 15 seconds on areas of the test formulation that had not been subjected to the afore-mentioned weight, using a new sheet each time. The time at which no more test composition is observed on the oil-absorbing paper is determined to be the Bioskin dry up time of the test formulation.

### Hand Dry Up Time

The hand dry up time is the same as the Bioskin dry up time described above except that the test formulation is applied on the dorsal side of the hand, instead of on the Bioskin substrate.

### Adhesion Peel Force Per Unit Length

This test method for adhesive force was developed in accordance with ASTM C794 Adhesion-in-Peel of Elastomeric Joint Sealants. Instron 3342 single column tension/ compression testing system (Instron, Norwood, MA) with 100N load cell (Instron #2519-103) mounted with extension grip geometry may be used, with polypropylene sheet of 1/32″ thickness as the test substrate. Other similar equipment and other soft, flexible test substrates can also be used to measure the peeling force. The materials and application of test composition to the selected substrates are described as follows: Apply the test composition onto the substrate, then gliding the glass slide back and forth along the spacer edges to deposit a smooth and uniform layer of test composition. Allow the test composition to sit untouched over the area at room temperature and ambient humidity for 24 hours. Then, place a silicone adhesive tape (Mepitac) of 0.75″ width on top of the film to fully cover the film surface on the polypropylene substrate. Allow the specimen to sit untouched over the area at room temperature and ambient humidity for 24 hours, before the measurement. For each material tested, at least 3 samples are measured, and average peeling force and standard deviation of the measurements are recorded. Partially peel the silicone tape-covered test specimen at one end by hand to separate enough of the silicone tape-covered film from the polypropylene substrate for effective grip by extension grip geometry mounts of the instrument. Secure each peeling side in its own instrument grip. Make sure the strips are clamped substantially parallel to the geometry. Perform the extension test at a rate of 1 mm/s until the two peeling strips separate completely from each other. Record the peeling force vs. time data. The sample’s average peeling force (N/m) is calculated by averaging the instantaneous force (N) measured by the instrument during the experiment normalized by the sample width (0.75″ or 0.019 m).

### EXAMPLE 1

Step 1A - Titration of Karstedt catalyst (Pt/DVDS) with additional divinyldisiloxane (DVDS) (with or without additional dilution from silicone fluid diluent PMS-1184). See Table 1A.

TABLE 1A

| Composition Reference No. | Pt/DVDS (g) | DVDS (g) | PMX-1184 (g) |
|---|---|---|---|
| AAA—034–50–A1 | 1 | 0 | 9 |
| AAA—034–50–A2 | 1 | 0.1125 | 9.8675 |
| AAA—034–50–A3 | 1 | 0.225 | 8.775 |
| AAA—034–50–A4 | 1 | 0.5625 | 8.4375 |
| AAA—034–50–A5 | 1 | 1.125 | 7.875 |
| AAA—034–50–A6 | 1 | 2.25 | 6.75 |
| AAA—034–50–A7 | 1 | 4.5 | 4.5 |
| AAA—034–50–A8 | 1 | 9 | 0 |
| AAA—034–50–A9 | 1 | 14 | 0 |
| AAA—034–50–A10 | 1 | 19 | 0 |
| AAA—034–50–A11 | 1 | 24 | 0 |

In Step 1A, all ingredients for each composition are added together in a glass vial and stirred with a vortex mixer.

Step 1B - Mixture of vinyl and hydride functional organopolysiloxanes (OPM-001) with Karstedt/DVDS titration from Step 1A. See Table 1B.

TABLE 1B

| | | | | |
|---|---|---|---|---|
| Composition Reference No. | OPM-003 | PMX-1184 | AAA-034–50–A (Pt/DVDS/PMX) 0.4 g | Note |
| AAA—034–50–B1 | 4 g | 5 g | AAA—034–50–A1 | Solidified over night |
| AAA—034–50–B2 | | | AAA—034–50–A2 | Solidified over night |
| AAA—034–50–B3 | | | AAA—034–50–A3 | Solidified over night |
| AAA—034–50–B4 | | | AAA—034–50–A4 | Solidified over night |
| AAA—034–50–B5 | | | AAA—034–50–A5 | Solidified over night |
| AAA—034–50–B6 | | | AAA—034–50–A6 | Solidified over night |
| AAA—034–50–B7 | | | AAA—034–50–A7 | Solidified over night |
| AAA—034–50–B8 | | | AAA—034–50–A8 | Best: stability & cure |
| AAA—034–50–B9 | | | AAA—034–50–A9 | Not ideal cure behavior |
| AAA—034–50–B10 | | | AAA—034–50–A10 | Not ideal cure behavior |
| AAA—034–50–B11 | | | AAA—034–50–A11 | Not ideal cure behavior |

In Step 1B, all ingredients are added together in a glass vial and stirred with a vortex mixer. Composition AAA-034-50-B8 including AAA-034-50-A8 has the best stability and cure among the compositions listed in Table 1B.

Step 1C - The mixture of Step 1A and the mixture of vinyl and hydride functional organopolysiloxanes in the diluent (Step 1 Pilot A -55% OPM-003 mixed with 45% PMX-1184 silicone fluid) with Karstedt/DVDS at 1:9 ratio (AAA-034-50-A8) - with or without other functional excipients. See Table 1C.

TABLE 1C

| Composition Reference No. | AAA-034–50–A8 Pt:DVDS (1:9) | Step 1 Pilot A (g) | Nylon 10–I2 (g) | KSG-710 (g) | Glycerol (g) | VDM/VQM (g) |
|---|---|---|---|---|---|---|
| AAA—034–50–C1 | 1 g | 9 | 0 | | | |
| AAA—034–50–C2 | | 8.7 | 0.3 | 0 | | |
| AAA—034–50–C3 | | 8.7 | 0 | 0.3 | 0 | |
| AAA—034–50–C4 | | 8.4 | 0.3 | 0.3 | 0 | |
| AAA—034–50–C5 | | 8.0 | 0.3 | 0.3 | 0.4 | 0 |
| AAA—034–50–C6 | | 8.0 | 0 | 0.3 | 0.7 | 0 |
| AAA—034–50–C7 | | 8.0 | 0.5 | 0 | | VQM2050 0.5 |
| AAA—034–50–C8 | | 8.0 | 0.5 | | | VQM6 0.5 |
| AAA—034–50–C9 | | 8.0 | 0.5 | | | VDM200 0.5 |
| AAA—034–50–C10 | | 8.0 | 0.5 | | | VDM181–83 0.5 |

In Step 1C, all ingredients are added together in a glass vial and stirred with a vortex mixer and the resulting composition is applied to the skin.

The results of Step 1C are now described:

AAA-034-50-C1: The resulting film was thin and shiny, with a gritty texture. The film cured in 5 minutes and was not durable overnight.

AAA-034-50-C2: The addition of the nylon did not help with the shine, and the texture was gritty. The film cured in 5 minutes, and showed slightly more durability than AAA-034-50-C1 overnight.

AAA-034-50-C3: Addition of the KSG-710 resulted in a thicker film (similar to that experienced with the addition of Nylon), but also resulted in somewhat less durability.

AAA-034-50-C4: The results are similar to that of AAA-034-50-C2 and AAA-034-50-C3 with regard to shine and texture.

AAA-034-50-C5: The addition of glycerol helps to smooth and soften the film somewhat, but the texture remains gritty.

AAA-034-50-C6: The results are essentially the same as AAA-034-50-C5.

AAA-034-50-C7: The film is dry at 5 minutes. The resulting film is cohesive with still texture.

AAA-034-50-C8: The film is dry at 4 minutes. The resulting film is flaky upon removal with still texture.

AAA-034-50-C9: The film is dry at 6 minutes. The resulting film is cohesive with still texture.

AAA-034-50-C10: The film is dry at 6 minutes. The resulting film is flaky upon removal with still texture, although somewhat softer than AAA-034-50-C7, AAA-034-50-C8, and AAA-034-50-C9.

### EXAMPLE 2

The mixture of heterobifunctional orgopolysiloxane with Karstedt/DVDS at 1:9 ratio (AAA-034-50-A8). See Table 2.]

TABLE 2

| | | |
|---|---|---|
| Composition Reference No. | AAA-034–50–A8 Pt:DVDS (1:9) | C═C—PDM—SiH series (Gelest) 4.5 g |
| AAA-034–50–D1 | 0.5 g | HV-12 (phenyl) 4.5 g |
| AAA-034–50–D2 | | HV-15 (50 cSt, MW 2500) 4.5 g |
| AAA-034–50–D3 | | HV-22 (200 cSt, MW 10000) 4.5 g |
| AAA-034–50–D4 | | HV-31 (1000 cSt, MW 50000) 4.5 g |

All ingredients are added together in a glass vial and stirred with a vortex mixer.

For each of the compositions in Example 2, such compositions never set after 1 day, 7 days, and 1 month. All remained fluid after 1 month.

### EXAMPLE 3

The mixture of vinyl organopolysiloxane with different size and structure with Karstedt/DVDS at 1:9 ratio (AAA-034-50-A8) and XL-11 hydride. See Table 3.

TABLE 3

| | | | | |
|---|---|---|---|---|
| Composition Reference No. | AAA-034–50–A8 Pt:DVDS (1:9) | XL-11 | PMX-1184 | VS series 4 |
| AAA-034–50–D1 | 1 g | 1 g | 4 g | VS250 (0.22 MMOL/G) 4 g |
| AAA-034–50–D2 | | | | VS500 (0.15 mmol/g) 4 g |
| AAA-034–50–D3 | | | | VS1000 (0.11 mmol/g) 4 g |
| AAA-034–50–D4 | | | | VS5000 (0.06 mmol/g) 4 g |
| AAA-034–50–D5 | | | | VS10000 (0.05 mmol/g) 4 g |
| AAA-034–50–D6 | | | | VS65000 (0.03 mmol/g) 4 g |
| AAA-034–50–D7 | | | | VS165000 (0.015 mmol/g) |
| AAA-034–50–D8 | | | | VDM500 (1.3 mmol/g) 4 g |
| AAA-034–50–D9 | | | | VDM65000 (0.28 mmol/g) 4 g |
| AAA-034–50–D10 | | | | VDM181–83 4 g |
| AAA-034–50–D11 | | | | VQM6–6 KcP (0.22 mmol/g) 4 g |
| AAA-034–50–D12 | | | | VQM60–60 KcP (0.20 mmol/g) 4 g |
| AAA-034–50–D13 | | | | VQM1040–15 KcP (0.40 mmol/g) 4 g |
| AAA-034–50–D14 | | | | VQM2050–500 cP (1.1 mmol/g) 4 g |

All ingredients are added together in a glass vial and stirred with a vortex mixer and the resulting composition is applied to the skin (hand) and Bioskin.

The results of Example 3 are now described:

AAA-034-50-D1: Remains fluid after 1 week; turned to soft gel after 2 weeks. The hand — dry up time is 2.5 minutes and the Bioskin — dry up time is 5.5 minutes.

AAA-034-50-D2: A softer gel after 72 hours. The hand — dry up time is 2.5 minutes and the Bioskin — dry up time is 5 minutes.

AAA-034-50-D3: A soft gel after 72 hours. The hand — dry up time is 2.5 minutes and the Bioskin — dry up time is 5.5 minutes.

AAA-034-50-D4: A hard gel after 72 hours. The hand — dry up time is 3 minutes and the Bioskin — dry up time is 4.5 minutes.

AAA-034-50-D5: Dries sticky; a harder gel after 72 hours. The hand — dry up time is 2 minutes and the Bioskin — dry up time is 4.5 minutes.

AAA-034-50-D6: Dries sticky; solidified after 5.0 hours (gel). The hand — dry up time is 2.25 minutes and the Bioskin — dry up time is 7 minutes.

AAA-034-50-D7: Solidified after 0.5 hours. The hand — dry up time is 3 minutes and the Bioskin — dry up time is 5.5 minutes.

AAA-034-50-D8: Remains fluid after 48 hours. The hand — dry up time is 4.5 minutes and the Bioskin — dry up time is 10 minutes.

AAA-034-50-D9: Solidified after 18 hours. The hand — dry up time is 5 minutes and the Bioskin — dry up time is 9 minutes.

AAA-034-50-D10: Solidified after 48 hours (gel). The hand — dry up time is 6 minutes and the Bioskin — dry up time is 15 minutes.

AAA-034-50-D11: Solidified after 48 hours (gel). The hand — dry up time is 4.5 minutes and the Bioskin — dry up time is 8 minutes.

AAA-034-50-D12: Much thicker fluid after 48 hours. The hand — dry up time is 4 minutes and the Bioskin — dry up time is 10 minutes.

AAA-034-50-D13: Solidified after 48 hours (gel). The hand — dry up time is 3 minutes and the Bioskin — dry up time is 8 minutes.

AAA-034-50-D14: Remains fluid after 1 week; turned to soft gel after 2 weeks. The hand — dry up time is 2.5 minutes and the Bioskin — dry up time is 7 minutes.

### EXAMPLE 4

The mixture of branched hydride organopolysiloxane with different hydride density with Karstedt/DVDS at 1:9 ratio (AAA-034-50-A8) and VS250 (250cSt linear vinyl terminal organopolysiloxane). See Table 4.

TABLE 4

| | | | | |
|---|---|---|---|---|
| Composition Reference No. | AAA-034–50–A8 Pt:DVDS (1:9) | VS250 | PMX-1184 | VS series 4 g |
| AAA-034–50–F1 | 1 g | 4 g | 4 g | XL10 (7.55 mmol/g, 45 cSt) 1 g |
| AAA-034–50–F2 | | | | XL11 (4.35 mmol/g, 45 cSt) 1 g |
| AAA-034–50–F3 | | | | XL15 (3.15 mmol/g, 40 cSt) 1 g |
| AAA-034–50–F4 | | | | XL17 (1.95 mmol/g, 50 cSt) 1 g |
| AAA-034–50–F5 | | | | XL14 (1.10 mmol/g, 40 cSt) 1 g |

All ingredients are added together in a glass vial and stirred with a vortex mixer and the resulting composition is applied to the skin (hand) and Bioskin.

The results of Example 4 are now described.

All compositions remained fluid after having been stored in a freezer.

AAA-034-50-F1: The hand-dry up time is 2.5 minutes and the Bioskin-dry up time is 6 minutes.

AAA-034-50-F2: The hand-dry up time is 4.5 minutes and the Bioskin-dry up time is 6.25 minutes.

AAA-034-50-F3: The hand-dry up time is 4 minutes and the Bioskin-dry up time is 5 minutes.

AAA-034-50-F4: The hand-dry up time is 6 minutes and the Bioskin-dry up time is 7 minutes.

AAA-034-50-F5: The hand-dry up time is 9 minutes and the Bioskin-dry up time is 9 minutes.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

### EXAMPLE 5

A schematic representation of the solvent evaporation process is presented in FIG. 3. In this method a water insoluble encapsulating agent is dissolved in a water immiscible volatile organic solvent, e.g., dichloromethane or chloroform or disiloxane or isododecane, into which the catalyst is also dissolved or dispersed. The resulting solution is added dropwise to a stirring aqueous solution having a suitable stabilizer to form small polymer droplets containing the encapsulated material. The core material may also be dispersed or dissolved in this aqueous solution instead. After a reasonable aging time, the droplets are hardened to produce the corresponding polymer microcapsules. This hardening process is accomplished by removal of the solvent from the polymer droplets either by solvent evaporation (by heat or reduced pressure), or by solvent extraction (with a third liquid which is a precipitant).

### EXAMPLE 6

A schematic representation of the spray drying process is presented in FIG. 4. The catalyst to be encapsulated is added to the solvent (the ratio of catalyst to solvent may be optimized) and the mixture is homogenized. The encapsulating agent is added at this stage. This mixture is then fed into the spray dryer with circulating hot air and atomized, which can be made by different types of atomizers: pneumatic atomizer, pressure nozzle, spinning disk, fluid nozzle and sonic nozzle. The solvent is evaporated by hot air and the encapsulating agent encapsulates the catalyst. Small particles of the resulting microcapsules are deposited in the collection vessel where they are collected.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This international application claims priority under U.S. Pat. Application No.63/047,648, filed Jul. 2, 2020, and the entire contents of U.S. Pat. Application No.63/047,648 are incorporated herein by reference.

1. A composition for application to skin of a subject, wherein the composition comprising:
(a) an unsaturated organopolymer;
(b) a hydride functionalized polysiloxane; and
(c) an environment-responsive agent that is capable of transporting out of the composition or facilitates out-transport of one or more beneficial agents from the composition after the composition is applied to a subject.

2. The composition as claimed in claim 1, wherein the environment-responsive agent is capable of transporting out of the composition.

3. The composition as claimed in claim 2, wherein the environment-responsive agent is a volatile agent that is capable of transporting out of the composition after the composition is applied to a subject.

4. The composition as claimed in claim 2, wherein the unsaturated organopolymer is a vinyl functionalized organopolysiloxane.

5. A composition for application to skin of a subject, wherein the composition comprises:
a) a bifunctional organopolysiloxane polymer having one unsaturated group and one hydride group; and
b) an environment-responsive agent that is capable of transporting out of the composition or facilitates out-transport of one or more beneficial agents from the composition after the composition is applied to a subject.

6. The composition of claim 5, wherein the environment-responsive agent is capable of transporting out of the composition.

7. The composition of claim 6, wherein the environment-responsive agent is a volatile agent that is capable of transporting out of the composition after the composition is applied to a subject.

8. The composition of claim 2, wherein the environment-responsive agent is transported out in a controlled manner.

9. The composition of claim 2,wherein the environment-responsive agent is transferred out by exposure to radiative transfer of electomagetic waves.

10. The composition of claim 2, wherein the environment-responsive agent is transported out of the composition by convection.

11. The composition of claim 2, wherein the environment-responsive agent is transported out by diffusion.

12. The composition of claim 2, wherein the environment-responsive agent is transferred out by evaporation.

13. The composition of claim 2, wherein the environment-responsive agent is transferred out by applying pressure.

14. The composition of claim 2, wherein the environment-responsive agent is transferred out by exposure to a sound, chemical, heat or light.

15. The composition of claim 2, wherein the environment-responsive agent is transferred out by absorbing the environment-responsive agent into another phase.

16. The composition of claim 2, wherein the environment-responsive agent is transferred out by absorbing the environment-responsive agent into the skin of a subject.

17. The composition of claim 2, wherein the environment-responsive agent is transferred out by absorbing the environment-responsive agent into another ingredient forming a complex.

18. The composition of claim 2, wherein the environment-responsive agent is transferred out by heating the composition.

19. The composition of claim 2, wherein the environment-responsive agent is transferred out by cooling the composition.

20. The composition of claim 2, wherein the environment-responsive agent is transferred out by using heat generated with a blow-dry.

21-292. (canceled)
